(19) United States
(12) Patent Application Publication
(10) Pub. No.: US 2005/0181355 A1
(43) Pub. Date: Aug. 18, 2005

(54) Compositions and methods for the modulation of viral maturation

(76) Inventors: Tsvika Greener, Ness-Ziona (IL); Haim Moskowitz, Jerusalem (IL); Yuval Reiss, Kiriat-Ono (IL); Iris Alroy, Ness-Ziona (IL)

Correspondence Address: FISH & NEAVE IP GROUP;ROPES & GRAY LLP, ONE INTERNATIONAL PLACE, BOSTON, MA, 02110-2624, US

(21) Appl. No.: 10/485,225

(22) PCT Filed: Jul. 31, 2002
(86) PCT No.: PCT/US02/24589

Related U.S. Application Data
(60) Provisional application No. 60/308,958, filed on Jul. 31, 2001.
(60) Provisional application No. 60/345,846, filed on Nov. 9, 2001.

(51) Int. Cl. C12Q 1/70; C07H 21/04; C07K 14/16; C12N 15/867

(52) U.S. Cl. 435/5; 435/69.3; 435/320.1; 435/325; 435/456; 530/350; 536/23.72

(57) ABSTRACT

This application describes a family of nucleic acid sequences and proteins encoded thereby that play a role in viral maturation: the Alternate Viral Maturation Scaffolding Protein, or the AVMSP family of proteins.

### RELATED APPLICATIONS

This application claims the benefit of U.S. Provisional Application Nos. 60/308,958, filed Jul. 31, 2001, and 60/345,846, filed Nov. 9, 2001. The entire contents of these applications are herein incorporated by reference.

### BACKGROUND

Viral maturation requires the proteolytic processing of viral proteins, such as Gag, and the activity of the host proteins. It is believed that cellular machineries for exo/endocytosis and for ubiquitin conjugation may be involved in the maturation. In particular, the assembly and subsequent budding of retroviruses, rhabdoviruses, and filoviruses depends on the Gag polyprotein. After its synthesis, Gag is targeted to the plasma membrane where it induces budding of nascent virus particles.

The role of ubiquitin in virus assembly was suggested by Dunigan et al. (1988, Virology 165, 310, Meyers et al. 1991, Virology 180, 602), who observed that mature virus particles were enriched in unconjugated ubiquitin. More recently, it was shown that proteasome inhibitors suppress the release of HIV-1, HV-2 and virus-like particles derived from SIV and RSV Gag. Also, inhibitors affect Gag processing and maturation into infectious particles (Schubert et al 2000, PNAS 97, 13057, Harty et al. 2000, PNAS 97, 13871, Strack et al. 2000, PNAS 97, 13063, Patnaik et al. 2000, PNAS 97, 13069).

It is well known in the art that ubiquitin-mediated proteolysis is the major pathway for the selective, controlled degradation of intracellular proteins in eukaryotic cells. Ubiquitin modification of a variety of protein targets within the cell appears to be important in a number of basic cellular functions such as regulation of gene expression, regulation of the cell-cycle, modification of cell surface receptors, biogenesis of ribosomes, and DNA repair. One major function of the ubiquitin-mediated system is to control the half-lives of cellular proteins. The half-life of different proteins can range from a few minutes to several days, and can vary considerably depending on the cell-type, nutritional and environmental conditions, as well as the stage of the cell-cycle.

Targeted proteins undergoing selective degradation, presumably through the actions of a ubiquitin-dependent proteosome, are covalently tagged with ubiquitin through the formation of an isopeptide bond between the C-terminal glycyl residue of ubiquitin and a specific lysyl residue in the substrate protein. This process is catalyzed by a ubiquitin-activating enzyme (E1) and a ubiquitin-conjugating enzyme (E2), and in some instances may also require auxiliary substrate recognition proteins (E3s). Following the linkage of the first ubiquitin chain, additional molecules of ubiquitin may be attached to lysine side chains of the previously conjugated moiety to form branched multi-ubiquitin chains.

The conjugation of ubiquitin to protein substrates is a multi-step process. In an initial ATP requiring step, a thioester is formed between the C-terminus of ubiquitin and an internal cysteine residue of an E1 enzyme. Activated ubiquitin is then transferred to a specific cysteine on one of several E2 enzymes. Finally, these E2 enzymes donate ubiquitin to protein substrates. Substrates are recognized either directly by ubiquitin-conjugated enzymes or by associated substrate recognition proteins, the E3 proteins, also known as ubiquitin ligases.

### SUMMARY

It is proposed that a variety of proteins, including ubiquitin protein ligases and proteins involved in membrane trafficking, are recruited for the process of viral maturation (including, for example, assembly, budding and release) by direct or indirect interaction with viral proteins, for example Gag proteins. The ligase then ubiquitinates viral and/or cellular proteins that are part of the membrane remodeling machinery. For example, a number of Gag protein motifs such as PxxP, PxxY, PPXY and YxxL, are known to recruit proteins involved in viral maturation.

To this end, in certain embodiments, the invention provides a family of nucleic acid sequences and proteins encoded thereby that play a role in viral maturation: the Alternate Viral Maturation Scaffolding Protein, or the AVMSP family of proteins. Broadly, AVMSP polypeptides comprise a first domain or functional role and a second domain. The first domain or functional role is selected from the following: SH2, SH3, or membrane spanning (“membrane”), or functions as a receptor. A preferred AVMSP also comprises a second domain that is a RING domain. Accordingly, different categories of AVMSPs may be referred to as RING-SH3 proteins and nucleic acids, RING-SH2 proteins and nucleic acids, RING-membrane proteins and nucleic acids and RING-receptor polypeptides and nucleic acids. The first domain and second domain may be found in any order within the AVMSP sequence (i.e. the first domain need not be N-terminal to the second domain). It is understood that polypeptides that function as receptors will often have membrane or other domains. In certain embodiments AVMSP proteins comprise a C2 domain.

In further aspects, in cells infected with viruses that utilize a Gag-dependent pathway for assembly, budding and/or release, AVMSPs, act to assemble complexes of proteins that mediate release. AVMSP complexes may, for example, stimulate, ubiquitylation of certain proteins, stimulate membrane fusion, stimulate assembly of viral particles, or a combination of the preceding. As one of skill in the art can readily appreciate, any single AVMSP may form multiple different complexes at different times.

In additional aspects, the invention provides nucleic acid sequences and proteins encoded thereby, as well as probes derived from the nucleic acid sequences, antibodies directed to the encoded proteins, diagnostic methods for detecting cells infected with a virus, and assays for identifying agents having an antiviral activity.

In one aspect, the invention provides a RING-SH3 nucleic acid, such as an isolated nucleic acid comprising a nucleotide sequence which hybridizes under stringent conditions to a sequence encoding a RING-SH3 protein, such as a sequence of SEQ ID Nos: 1-3, or a sequence complementary thereto. In a related embodiment, the nucleic acid is at least about 80%, 90%, 95%, or 97-98%, or 100% identical to a sequence corresponding to at least about 12, at least about 15, at least about 25, or at least about 40 consecutive nucleotides up to the full length of one of SEQ ID Nos. 40-99 or a sequence complementary thereto or up to the full length of the gene of which said sequence is a fragment. In a further embodiment, the RING-SH3 nucleic acid comprises a nucleic acid encoding an amino acid sequence as set forth in SEQ ID Nos. 1-39 or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40 consecutive amino acids up to the full length of one of SEQ ID Nos: 1-39. In yet another embodiment, the RING-SH3 nucleic acid is an isolated nucleic acid encoding a polypeptide comprising a RING domain and an SH3 domain. In a preferred embodiment, the RING-SH3 nucleic acid is a PRT3 nucleic acid of SEQ ID NOs:40-44 or a functional variant thereof.

In a further aspect, the invention provides a RING-SH2 nucleic acid, such as an isolated nucleic acid comprising a nucleotide sequence which hybridizes under stringent conditions to a sequence encoding a RING-SH2 protein, such as a sequence of SEQ ID Nos: 45-46, or a sequence complementary thereto. In a related embodiment, the nucleic acid is at least about 80%, 90%, 95%, or 97-98%, or 100% identical to a sequence corresponding to at least about 12, at least about 15, at least about 25, or at least about 40 consecutive nucleotides up to the full length of one of SEQ ID Nos.45-46, or a sequence complementary thereto or up to the full length of the gene of which said sequence is a fragment. In a further embodiment, the RING-SH2 nucleic acid comprises a nucleic acid encoding an amino acid sequence as set forth in SEQ ID Nos. 4-5, or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40 consecutive amino acids up to the full length of one of SEQ ID NOS: 4-5. In yet another embodiment, the RING-SH2 nucleic acid is an isolated nucleic acid encoding a polypeptide comprising a RING domain and an SH2 domain.

In a further aspect, the invention provides a RING-membrane nucleic acid, such as an isolated nucleic acid comprising a nucleotide sequence which hybridizes under stringent conditions to a sequence encoding a RING-membrane protein, such as a sequence of SEQ ID Nos: 47-56, or a sequence complementary thereto. In a related embodiment, the nucleic acid is at least about 80%, 90%, 95%, or 97-98%, or 100% identical to a sequence corresponding to at least about 12, at least about 15, at least about 25, or at least about 40 consecutive nucleotides up to the full length of one of SEQ ID Nos. 47-56, or a sequence complementary thereto or up to the fill length of the gene of which said sequence is a fragment. In a further embodiment, the RING-membrane nucleic acid comprises a nucleic acid encoding an amino acid sequence as set forth in SEQ ID Nos. 6-15, or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40 consecutive amino acids up to the full length of one of SEQ ID NOS: 6-15. In yet another embodiment, the RING-membrane nucleic acid is an isolated nucleic acid encoding a polypeptide comprising a RING domain and a membrane domain.

In a further aspect, the invention provides a RING-receptor nucleic acid, such as an isolated nucleic acid comprising a nucleotide sequence which hybridizes under stringent conditions to a sequence encoding a RING-receptor protein, such as a sequence of SEQ ID Nos: 57-72, or a sequence complementary thereto. In a related embodiment, the nucleic acid is at least about 80%, 90%, 95%, or 97-98%, or 100% identical to a sequence corresponding to at least about 12, at least about 15, at least about 25, or at least about 40 consecutive nucleotides up to the full length of one of SEQ ID Nos. 57-72, or a sequence complementary thereto or up to the full length of the gene of which said sequence is a fragment. In a further embodiment, the RING-receptor nucleic acid comprises a nucleic acid encoding an amino acid sequence as set forth in SEQ ID Nos. 16-31, or a nucleic acid complement thereof. In a related embodiment, the encoded amino acid sequence is at least about 80%, 90%, 95%, or 97-98%, or 100% identical to a sequence corresponding to at least about 12, at least about 15, at least about 25, or at least about 40 consecutive amino acids up to the full length of one of SEQ ID NOS: 16-31. In yet another embodiment, the RING-receptor nucleic acid is an isolated nucleic acid encoding a polypeptide comprising a RING domain and a receptor domain.

In one embodiment, the invention provides an expressible RING-SH3, RING-SH2, RING-membrane or RING-receptor nucleic acid operably linked to a transcriptional regulatory sequence, rendering the expressible nucleotide sequence suitable for use as an expression vector. In another embodiment, the nucleic acid may be included in an expression vector capable of replicating in a prokaryotic or eukaryotic cell. In a related embodiment, the invention provides a host cell transfected with the expression vector.

In yet another embodiment, the invention provides a substantially pure RING-SH3, RING-SH2, RING-membrane or RING-receptor nucleic acid which hybridizes under stringent conditions to a nucleic acid probe corresponding to at least about 12, at least about 15, at least about 25, or at least about 40 consecutive nucleotides up to the full length of one of SEQ ID Nos. 40-72, or a sequence complementary thereto or up to the full length of the gene of which said sequence is a fragment. The invention also provides an antisense oligonucleotide analog which hybridizes under stringent conditions to at least 12, at least 25, or at least 50 consecutive nucleotides of one of SEQ ID NOS 40-72, or a sequence complementary thereto.

In another embodiment, the invention provides a probe/primer comprising a substantially purified RING-SH3 oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ ID Nos.40-44, or a sequence complementary thereto.

In another embodiment, the invention provides a probe/primer comprising a substantially purified RING-SH2 oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ ID Nos.4546, or a sequence complementary thereto.

In another embodiment, the invention provides a probe/primer comprising a substantially purified RING-membrane oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ D Nos.47-56, or a sequence complementary thereto.

In another embodiment, the invention provides a probe/primer comprising a substantially purified RING-receptor oligonucleotide, said oligonucleotide containing a region of nucleotide sequence which hybridizes under stringent conditions to at least about 12, at least about 15, at least about 25, or at least about 40 consecutive nucleotides of sense or antisense sequence selected from SEQ ID Nos.57-72, or a sequence complementary thereto.

In preferred embodiments, a probe as described above selectively hybridizes with a target nucleic acid. In another embodiment, the probe may include a label group attached thereto and able to be detected. The label group may be selected from radioisotopes, fluorescent compounds, enzymes, and enzyme co-factors. The invention further provides arrays of at least about 10, at least about 25, at least about 50, or at least about 100 different probes as described above attached to a solid support.

In another aspect, the invention provides polypeptides. In one embodiment, the invention pertains to a RING-SH3 polypeptide including an amino acid sequence encoded by a nucleic acid comprising a nucleotide sequence which hybridizes under stringent conditions to a sequence of SEQ ID Nos. 40-44, or a sequence complementary thereto, or a fragment comprising at least about 25, or at least about 40 amino acids thereof.

In another aspect, the invention provides polypeptides. In one embodiment, the invention pertains to a RING-SH2 polypeptide including an amino acid sequence encoded by a nucleic acid comprising a nucleotide sequence which hybridizes under stringent conditions to a sequence of SEQ ID Nos. 45-46, or a sequence complementary thereto, or a fragment comprising at least about 25, or at least about 40 amino acids thereof.

In another aspect, the invention provides polypeptides. In one embodiment, the invention pertains to a RING-membrane polypeptide including an amino acid sequence encoded by a nucleic acid comprising a nucleotide sequence which hybridizes under stringent conditions to a sequence of SEQ ID Nos. 47-56, or a sequence complementary thereto, or a fragment comprising at least about 25, or at least about 40 amino acids thereof.

In another aspect, the invention provides polypeptides. In one embodiment, the invention pertains to a RING-receptor polypeptide including an amino acid sequence encoded by a nucleic acid comprising a nucleotide sequence which hybridizes under stringent conditions to a sequence of SEQ ID Nos. 57-72, or a sequence complementary thereto, or a fragment comprising at least about 25, or at least about 40 amino acids thereof.

In a preferred embodiment, the polypeptide is identical with or homologous to a RING-SH3, RING-SH2, RING-membrane or RING-receptor protein represented by SEQ ID Nos: 1-31. For instance, a polypeptide preferably has an amino acid sequence at least 70% homologous to a polypeptide represented by any of SEQ ID Nos: 1-31, though polypeptides with higher sequence homologies of, for example, 80%, 90% or 95% are also contemplated. The polypeptide can comprise a full length protein, such as represented in the sequence listings, or it can comprise a fragment of, for instance, at least 5, 10, 20, 50, 100, 150 or 200 amino acids in length.

In another preferred embodiment, the invention features a purified or recombinant polypeptide fragment of a RING-SH3, RING-SH2, RING-membrane or RING-receptor polypeptide, which polypeptide has the ability to modulate, e.g., mimic or antagonize, an activity of a wild-type RING-SH3, RING-SH2, RING-membrane or RING-receptor polypeptide. Preferably, the polypeptide fragment comprises a sequence identical or homologous to an amino acid sequence designated in one of SEQ ID Nos: 1-31.

Moreover, as described below, the RING-SH3, RING-SH2, RING-membrane or RING-receptor polypeptide can be either an agonist (e.g. mimics), or alternatively, an antagonist of a biological activity of a naturally occurring form of the protein, e.g., the polypeptide is able to modulate the intrinsic biological activity of a RING-SH3, RING-SH2, RING-membrane or RING-receptor complex, such as an enzymatic activity, binding to other cellular components, cellular compartmentalization, and the like.

The subject proteins can also be provided as chimeric molecules, such as in the form of fusion proteins. For instance, the AVMSP can be provided as a recombinant fusion protein which includes a second polypeptide portion, e.g., a second polypeptide having an amino acid sequence unrelated (heterologous) to the AVMSP, e.g. the second polypeptide portion is glutathione-S-transferase, e.g. the second polypeptide portion is an enzymatic activity such as alkaline phosphatase, e.g. the second polypeptide portion is an epitope tag, e.g. the second polypeptide is an affinity purification tag.

Yet another aspect of the present invention concerns an immunogen comprising an AVMSP in an immunogenic preparation, the immunogen being capable of eliciting an immune response specific for said AVMSP; e.g. a humoral response, e.g. an antibody response; e.g. a cellular response. In preferred embodiments, the immunogen comprising an antigenic determinant, e.g. a unique determinant, from a protein represented by one of SEQ ID Nos. 1-39.

In yet another aspect, this invention provides antibodies immunoreactive with one or more AVMSPs. In one embodiment, antibodies are specific for a RING domain, an SH3 domain, a SH2 domain, or a receptor domain and preferably the domain is part of an AVMSP. In a more specific embodiment, the domain is part of an amino acid sequence set forth in SEQ ID Nos. 1-39. In another embodiment, the antibodies are immunoreactive with one or more proteins having an amino acid sequence that is at least 80% identical to an amino acid sequence as set forth in SEQ ID Nos. 1-39. In other embodiments, an antibody is immunoreactive with one or more proteins having an amino acid sequence that is 85%, 90%, 95%, 98%, 99% or identical to an amino acid sequence as set forth in SEQ ID Nos. 1-39.

In an additional aspect, the invention provides complexes comprising an AVMSP and an AVMSP associated protein (an “AVMSP-AP”). In one embodiment, the invention provides an isolated protein complex comprising a RING-SH3, RING-SH2, RING-membrane or RING-receptor polypeptide in combination with at least one polypeptide selected from the group consisting of: a RING-SH3, a RING-SH2, a RING-membrane, a RING-receptor, a Gag protein, a Gag late domain, PI3K, actin, myosin, Hsp60, Hsp70, Hsp90, STAM1, STAM2A, STAM2B, VHS-UIM, a GTPase, an E2 enzyme, tsg101, a cullin and a clathrin. In another embodiment, the isolated protein complex comprises a RING-SH3, RING-SH2, RING-membrane or RING-receptor polypeptide and a Gag protein in combination with a polypeptide selected from the group consisting of: a RING-SH3, a RING-SH2, a RING-membrane, a RING-receptor, PI3K, actin, myosin, Hsp60, Hsp70, Hsp90, STAM1, STAM2A, STAM2B, VHS-UIM, a GTPase, an E2 enzyme, tsg101, a cullin and a clathrin.

In yet another embodiment, the invention provides an isolated protein complex comprising an AVMSP polypeptide and a HIV Gag protein in combination with a polypeptide selected from the group consisting of: RING-SH3, RING-SH2, RING-membrane, RING-receptor, PI3K, actin, myosin, Hsp60, Hsp70, Hsp90, STAM1, STAM2A, STAM2B, VHS-UIM, a GTPase, an E2 enzyme, tsg101, a cullin and a clathrin. The invention also provides an isolated protein complex comprising a RING-SH3, RING-SH2, RING-membrane or RING-receptor polypeptide and an HIV Gag protein in combination with a polypeptide selected from the group consisting of: RING-SH3, RING-SH2, RING-membrane or RING-receptor, PI3K, actin, myosin, Hsp60, Hsp70, Hsp90, STAM1, STAM2A, STAM2B, VHS-UIM, a GTPase, an E2 enzyme, tsg101, a cullin and a clathrin.

In yet another aspect, the invention provides an assay for screening test compounds for inhibitors, or alternatively, potentiators, of an interaction between an AVMSP and an AVMSP-AP. In the case of a RING-SH3 polypeptide, exemplary associated proteins (“RING-SH3-AP”) include RING-SH3 proteins, E2 proteins (e.g. tsg101), Gag proteins, proteins comprising an L-domain, phosphatidylinositol-3-kinases, as well as proteins involved in endocytosis such as clathrins, actins, myosins, HSP60, HSP70, HSP90, STAM1, STAM2A, and STAM2B. An exemplary method includes the steps of (i) combining AVMSP-AP (e.g. a RING-SH3-AP, RING-SH2-AP, RING-membrane-AP or RING-receptor-AP), an AVMSP, and a test compound, e.g., under conditions (including, as desired, the addition of additional proteins) wherein, but for the test compound, the AVMSP and an AVMSP-AP are able to interact; and (ii) detecting the formation of a complex which includes the AVMSP and an AVMSP-AP. A statistically significant change, such as a decrease, in the formation of the complex in the presence of a test compound (relative to what is seen in the absence of the test compound) is indicative of a modulation, e.g., inhibition, of the interaction between the AVMSP and an AVMSP-AP. Similar assays may employ preformed AVMSP-AVMSP-AP complexes to assess the ability of the test compound to destabilize or stabilize the complex.

In yet another aspect, the invention provides cells carrying a recombinant form of an AVMSP nucleic acid, often included on a vector. In further embodiments, cells carry a recombinant form of an AVMSP nucleic acid and a recombinant form of a nucleic acid encoding a Gag protein and/or a polypeptide comprising an L domain motif, such as P(T/S)AP, PPxY or YxxL. In certain aspects, the cells are bacterial, and in other aspects the cells are eukaryotic cells, preferrably a mammalian cell line.

The practice of the present invention will employ, unless otherwise indicated, conventional techniques of cell biology, cell culture, molecular biology, transgenic biology, microbiology, recombinant DNA, and immunology, which are within the skill of the art.

Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### BRIEF DESCRIPTION OF THE DRAWINGS

FIG. 1: Human PRT3 amino acid sequence (SEQ ID NO: 1)

FIG. 2A: N terminus protein fragment of hPRT3 (public gi: 10432612; SEQ ID NO:2)

FIG. 2B: C terminus protein fragment of hPRT3 (public gi: 7959249; SEQ ID NO:39)

FIGS. 3-39 show amino acid sequences for examples of AVMSP polypeptides.

FIGS. 40-78 show nucleic acid sequences encoding examples of AVMSP polypeptides.

FIG. 79: Human PRT3 full mRNA, annotated sequence

FIG. 80: Domain analysis of human PRT3

FIG. 81: Diagram of human PRT3 nucleic acids. The diagram shows the full-length PRT3 gene and the position of regions amplified by RT-PCR or targeted by siRNA used in FIG. 11.

FIG. 82: Knockdown of PRT3 mRNA by siRNA duplexes. HeLa SS-6 cells were transfected with siRNA against Lamin A/C (lanes 1, 2) or PRT3 (lanes 3-10). PRT3 siRNA was directed against the coding region (153—lanes 3,4; 155—lanes 5,6) or the 3′UTR (157-lanes 7, 8; 159—lanes 9, 10). Cells were harvested 24 hours post-transfection, RNA extracted, and PRT3 mRNA levels compared by RT-PCR of a discrete sequence in the coding region of the PRT3 gene (see FIG. 10). GAPDH is used an RT-PCR control in each reaction.

FIG. 83: PRT3 affects the release of VLP from cells. A) Posphohimages of SDS-PAGE gels of immunoprecipitations of ³⁵S pulse-chase labeled Gag proteins are presented for cell and viral lysates from transfected HeLa cells that where either untreated or treated with PRT3 RNAi (50 nM for 48 hours). The time during the chase period (1,2,3,4 and 5 after the pulse) are presented from left to right for each image.

FIG. 84: Release of VLP from cells at steady state. Hela cells were transfected with an “HV-encoding plasmid and siRNA. Lanes 1, 3 and 4 were transfected with wild-type HIV-encoding plasmid. Lane 2 was transfected with an HI-V-encoding plasmids which contains a point mutation in p6 (PTAP to ATAP). Control siRNA (lamin A/C) was transfected to cells in lanes 1 and 2. siRNA to Tsg101 was transfected in lane 4 and siRNA to PRT3 in lane 3.

FIG. 85: Exemplary HIV-1 Gag Nucleic Acid Sequence (Acc. No. NC-001802) [SEQ ID NO:80]

FIG. 86: Exemplary HIV-1 Gag Amino Acid Sequence (Acc. No. NP_{—}057850) [SEQ ID NO:81]

FIG. 87: Exemplary HIV-I p6 Amino Acid Sequence (SEQ ID NO:82)

FIG. 88: Representative consensus terms for domains

FIG. 89: Comparative Sequence Analysis—Amino Acid Grouping

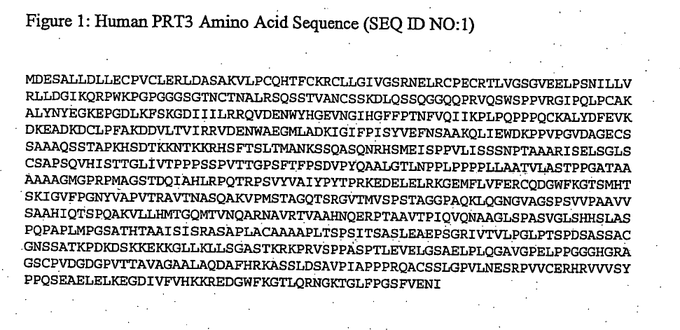

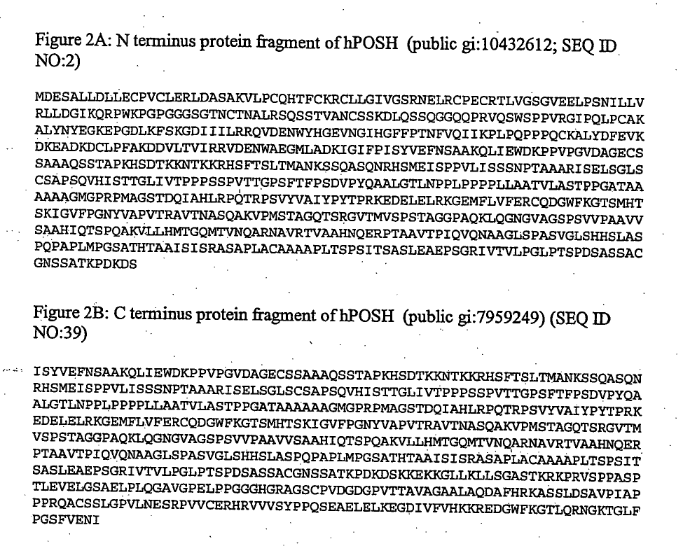

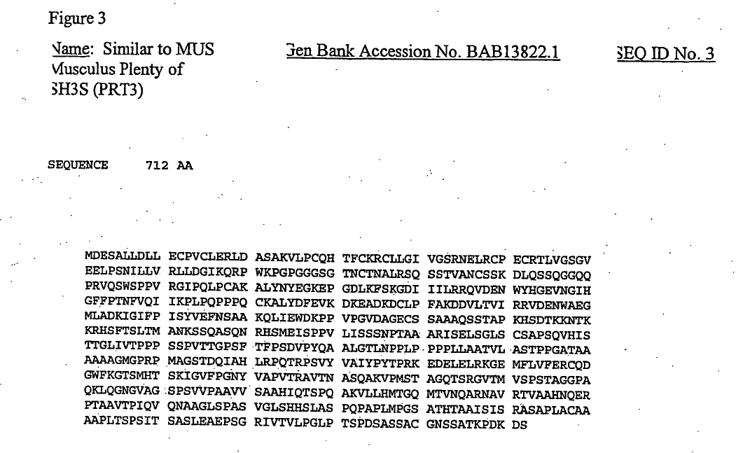

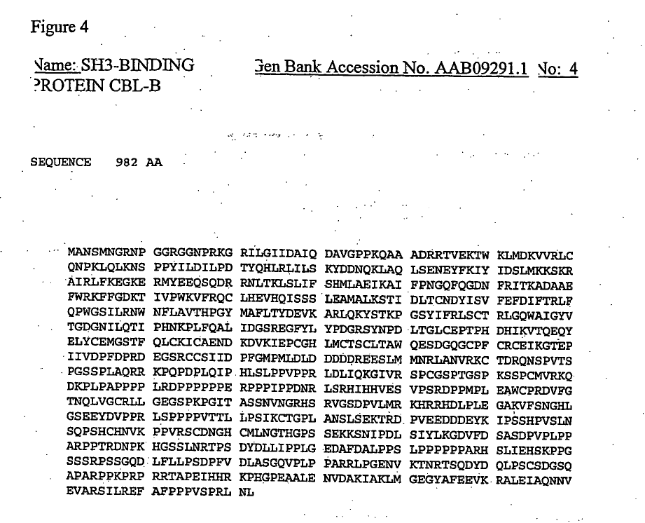

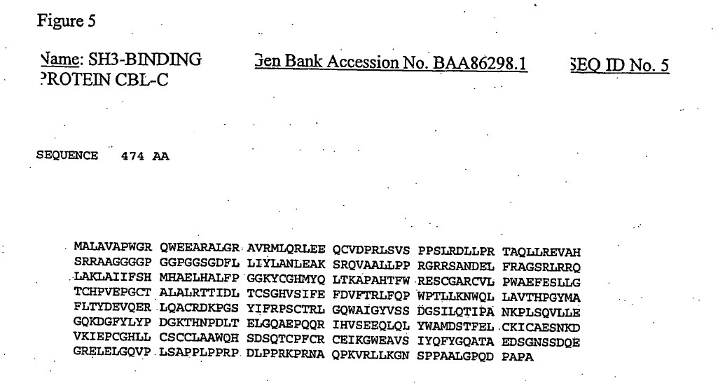

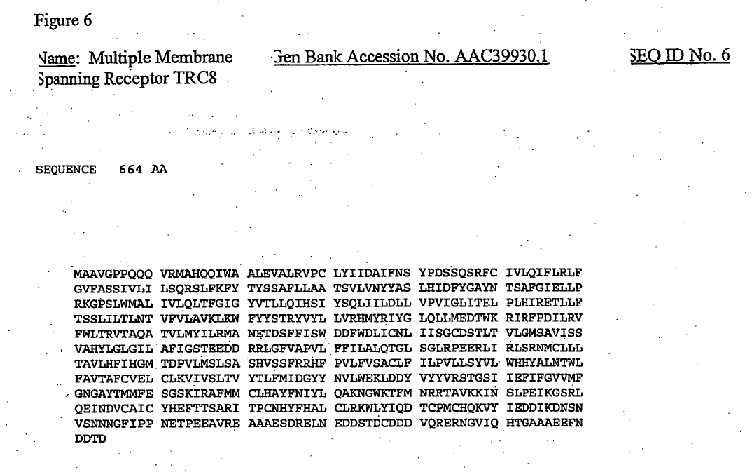

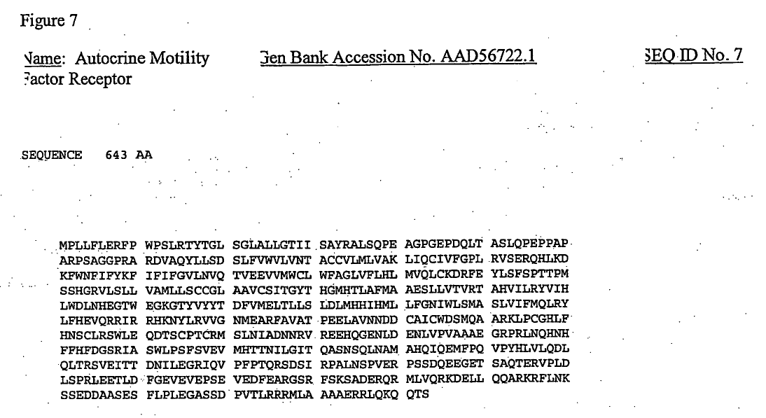

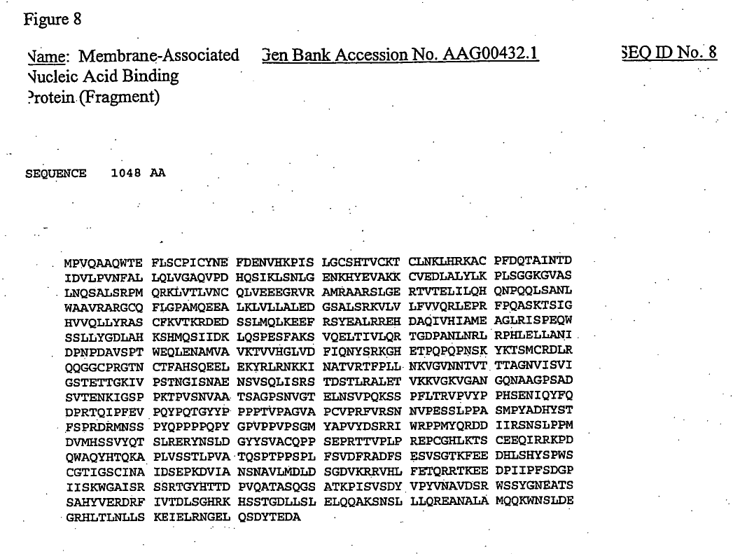

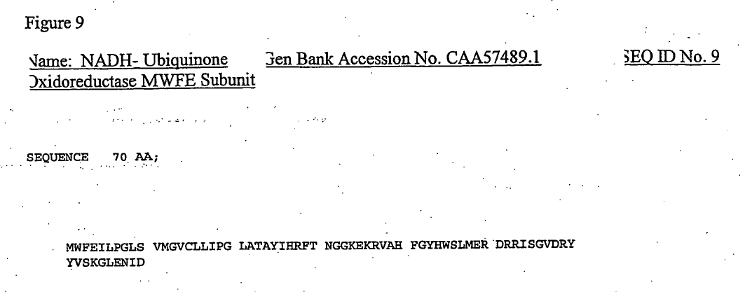

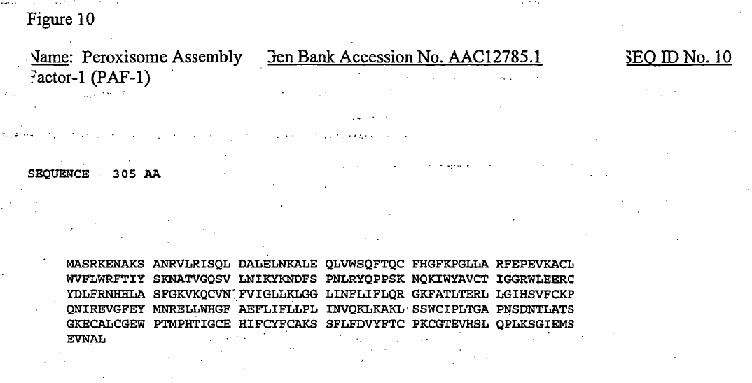

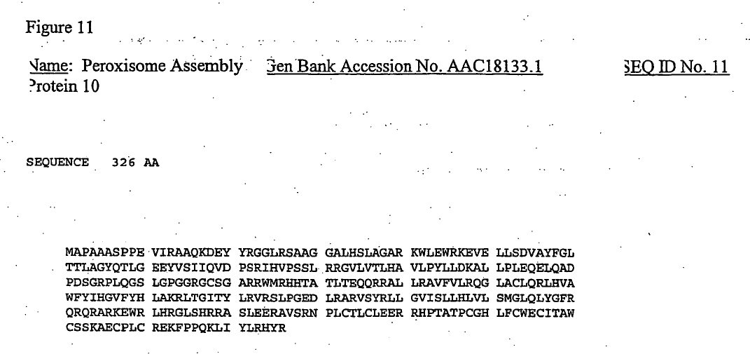

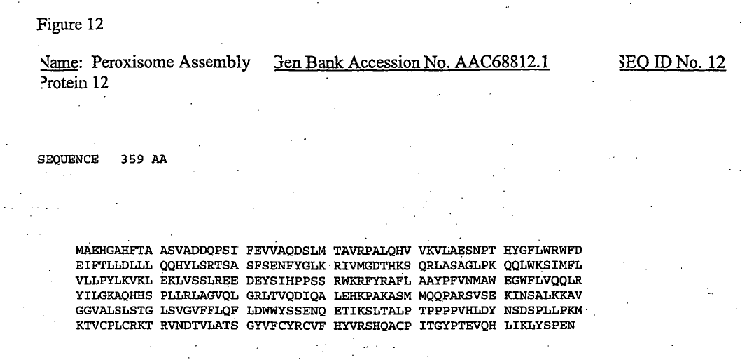

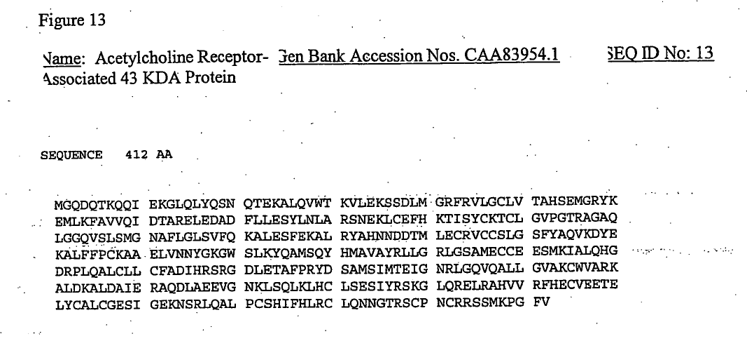

### DETAILED DESCRIPTION OF THE INVENTION

1. Definitions

The term “Alternate Viral Maturation Scaffolding Protein” or “AVMSP” is used herein to indicate a polypeptide comprising a first domain or functionality and a second domain. The first domain or functionality is selected from the group consisting of: an SH2 domain, an SH3 domain, a membrane spanning domain, and a receptor functionality (meaning the protein, or a portion thereof functions as a receptor). An AVMSP also comprises a second domain that is, for the purposes of this application, a RING domain. The first domain and second domain may be found in any order within the AVMSP sequence (i.e. the first domain need not be N-terminal to the second domain). The term AVMSP encompasses proteins represented as SEQ ID Nos. 1-39. It is understood that polypeptides having functional roles, such as receptors, may farther comprise membrane or other domains. In other embodiments, AVMSPs may further comprise one or more C2 domains. In cells infected with viruses that utilize a Gag-dependent pathway for budding and release, an AVMSP may act to assemble complexes of proteins that mediate release. AVMSP complexes may stimulate ubiquitylation of certain proteins or stimulate membrane fusion or both. Any single AVMSP may form multiple different AVMSP complexes at different times.

The term “Alternate Viral Maturation Scaffolding Protein-Associated Protein” (AVMSP-AP) refers to protein capable of interacting and/or binding to an AVMSP. Generally, the AVMSP-AP may interact directly or indirectly with the AVMSP. Examples of these proteins include for example the “Late domain” or “L domain”, which is a small portion of a Gag protein that promotes efficient release of virion particles from the membrane of the host cell. L domains typically comprise one or more short motifs (L motifs). Exemplary sequences include: P(T/S)AP, PxxL, PPxY (eg. PPPY), YxxL (eg. YPDL), PxxP. Other exemplary AVMSP-APs are provided throughout.

The term “binding” refers to a direct association between two molecules, due to, for example, covalent, electrostatic, hydrophobic, ionic and/or hydrogen-bond interactions under physiological conditions.

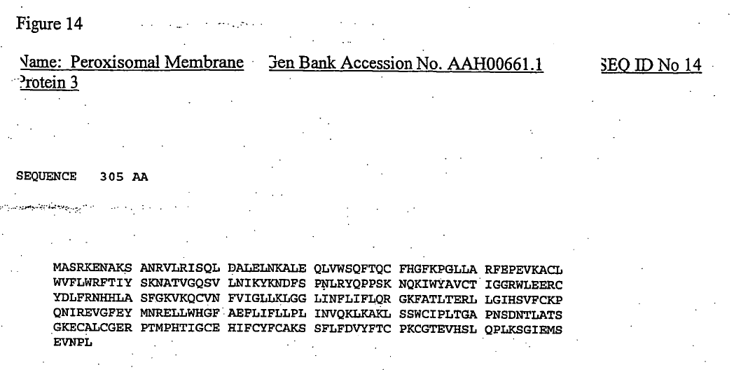

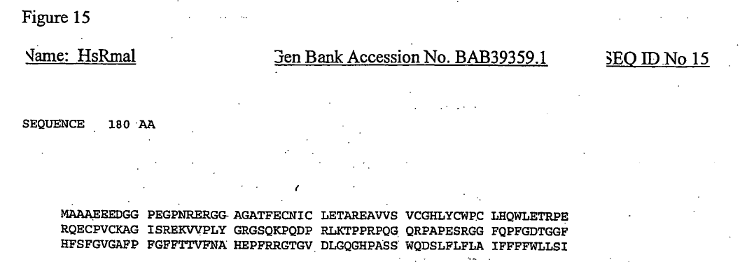

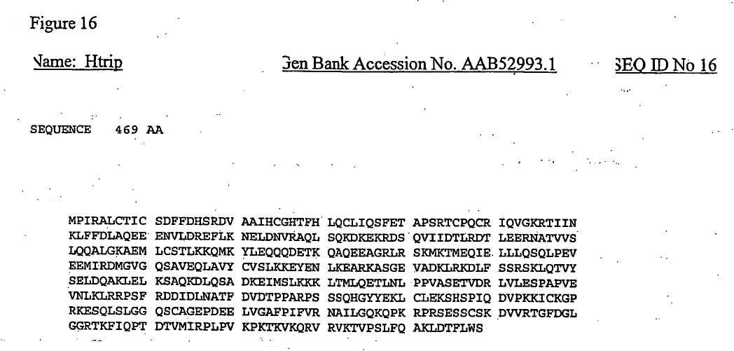

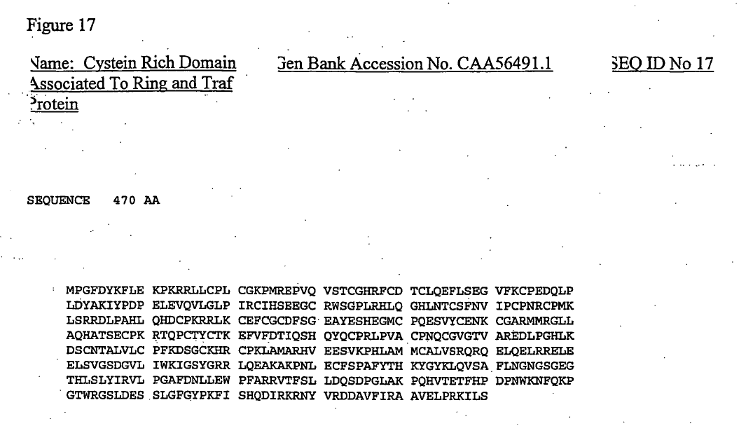

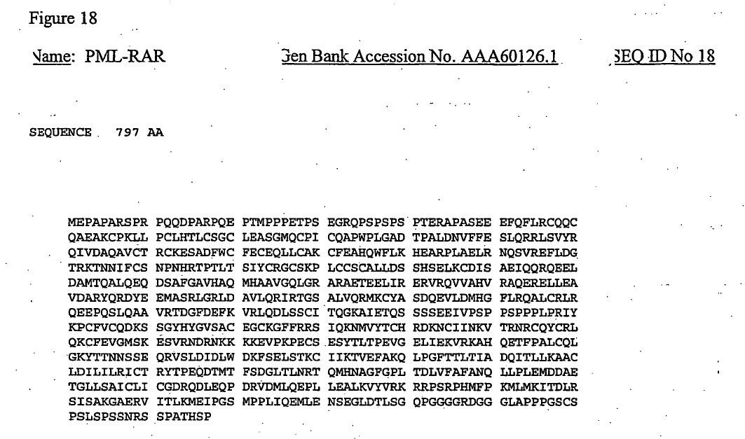

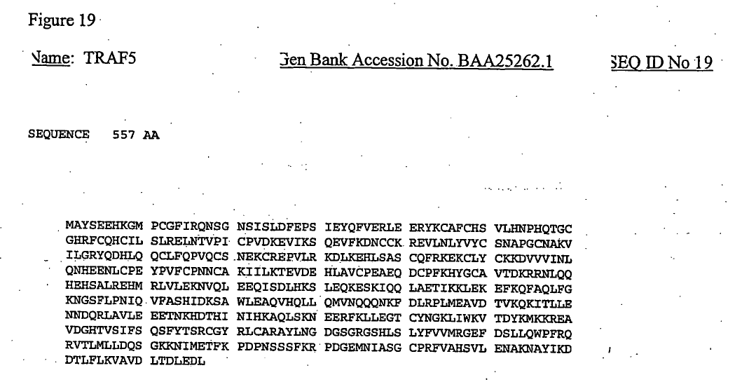

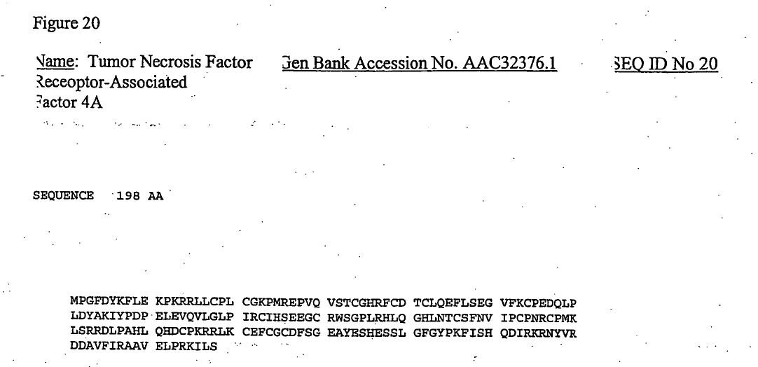

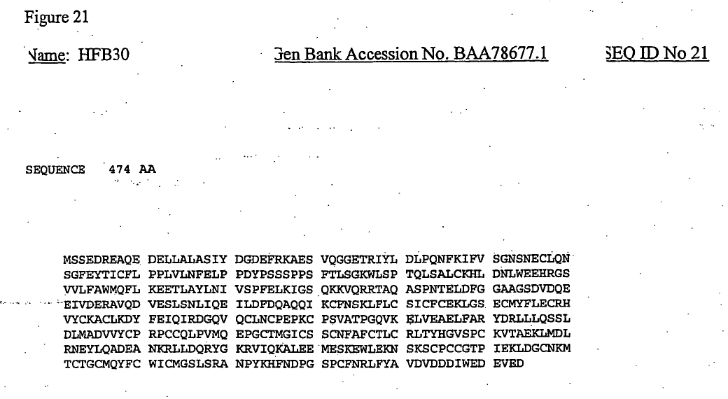

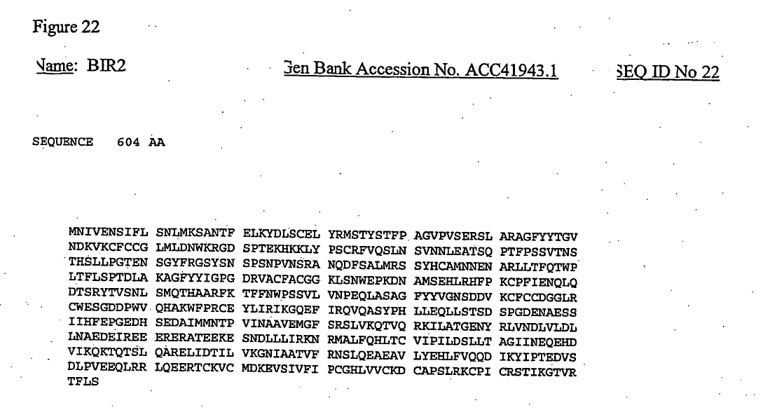

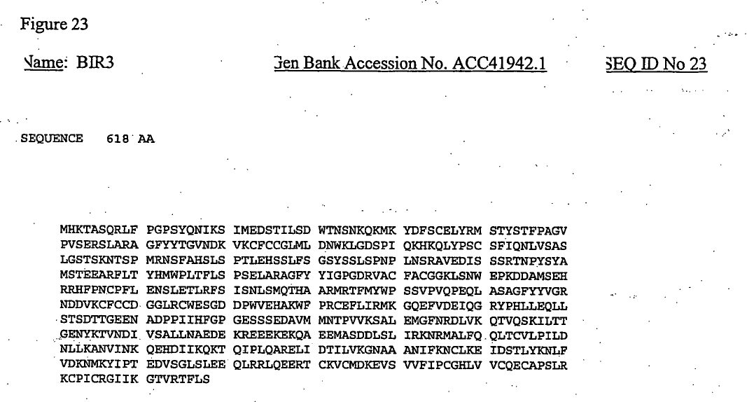

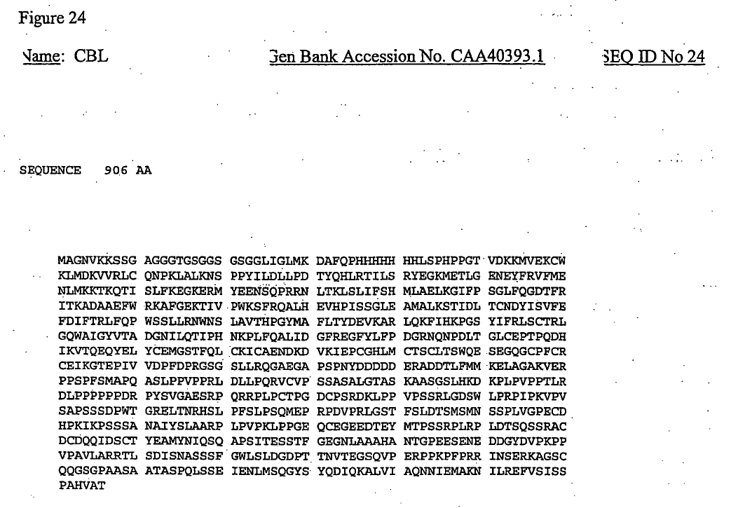

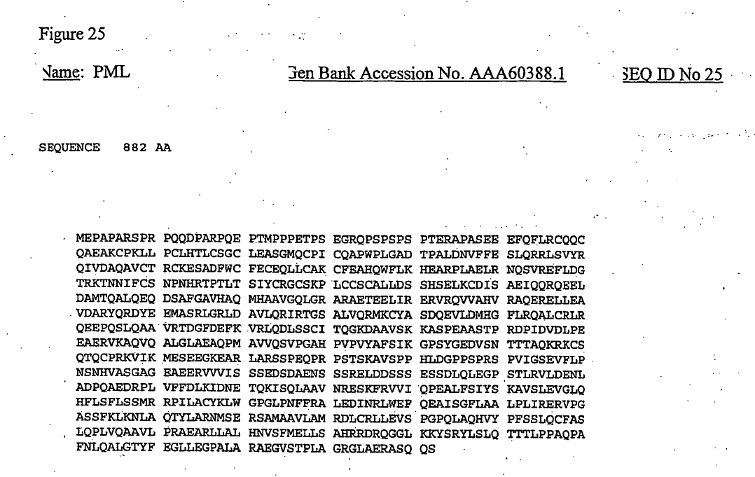

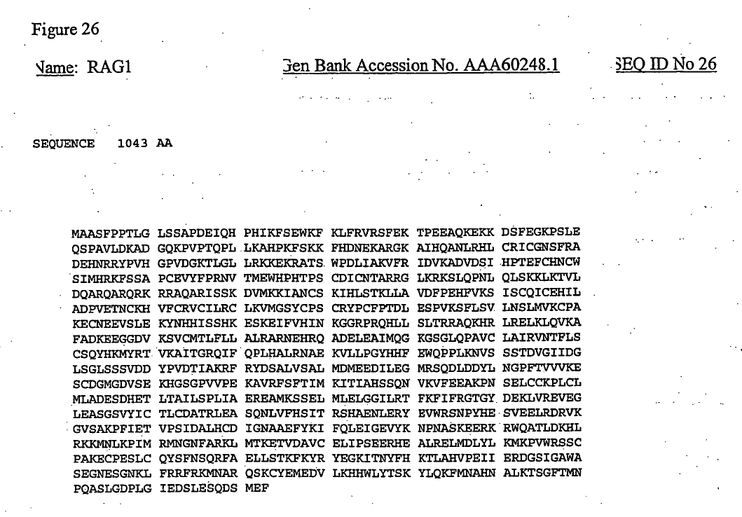

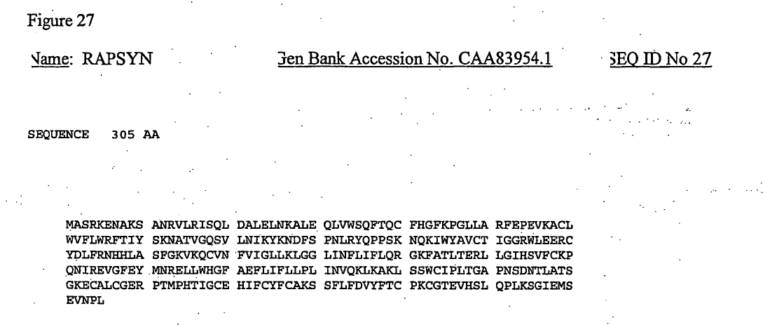

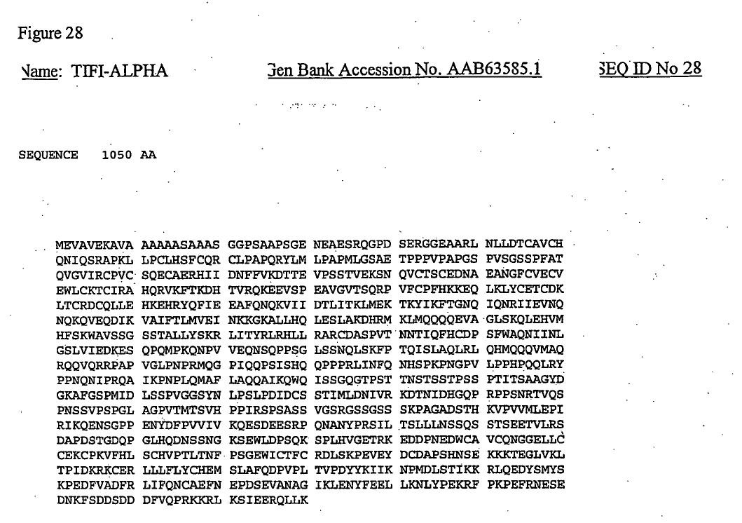

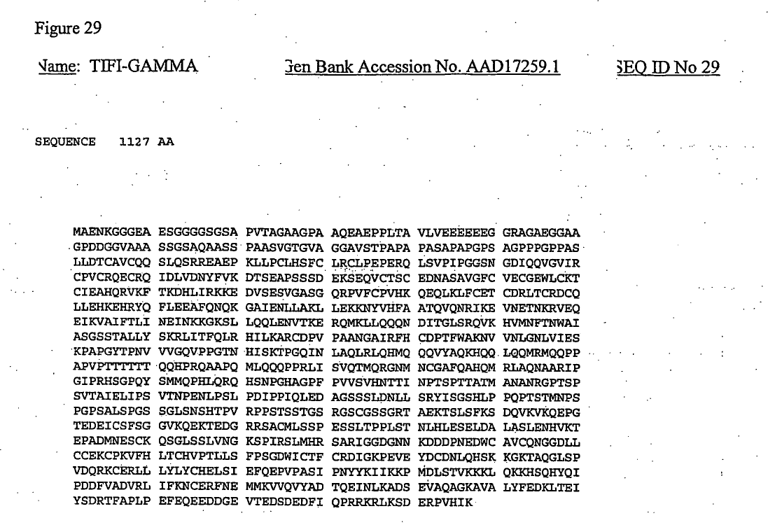

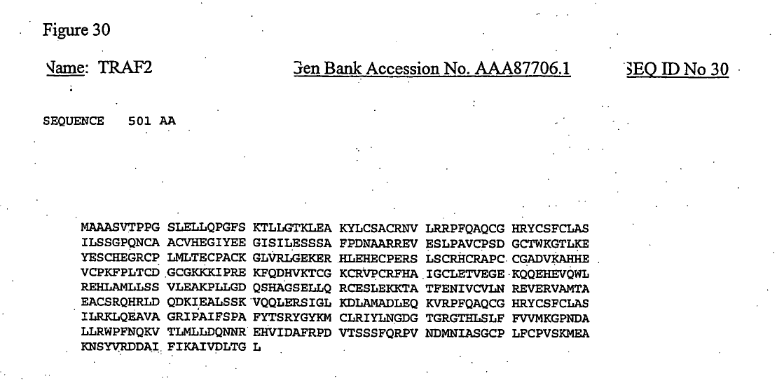

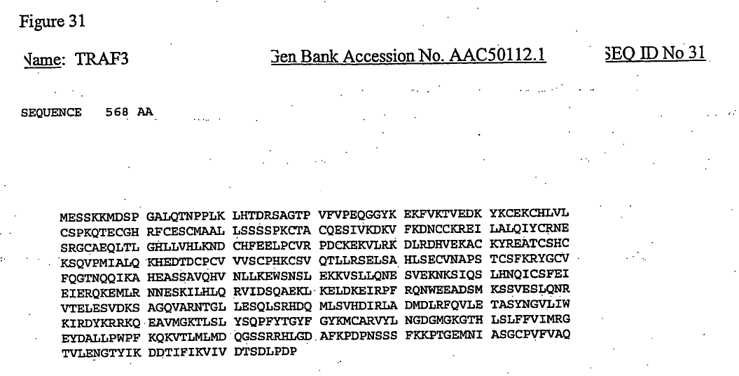

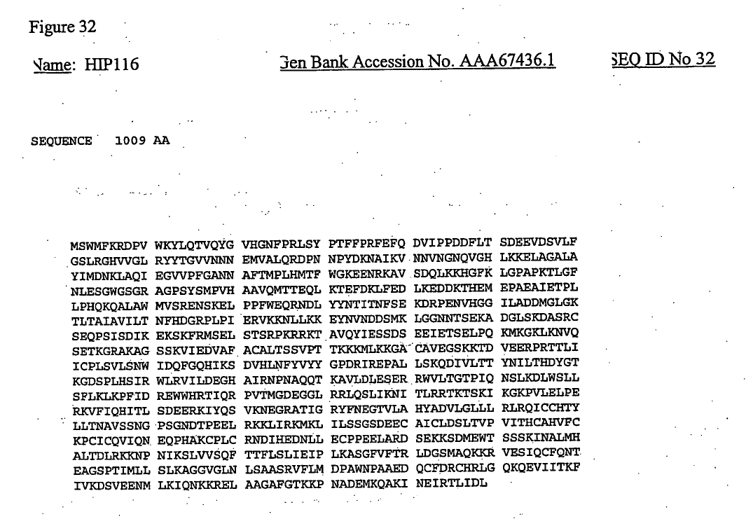

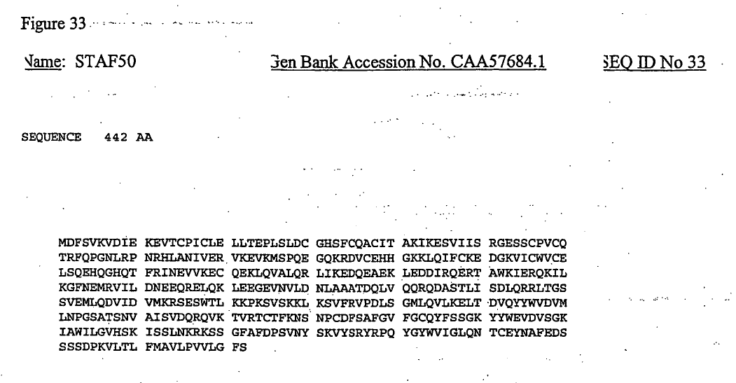

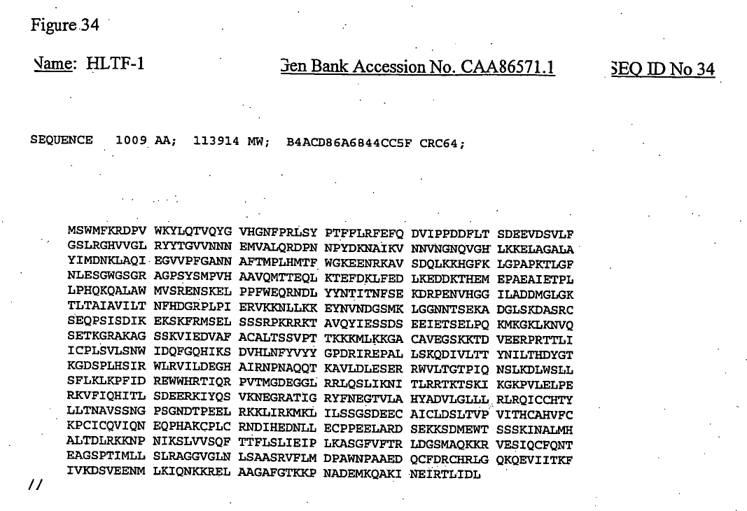

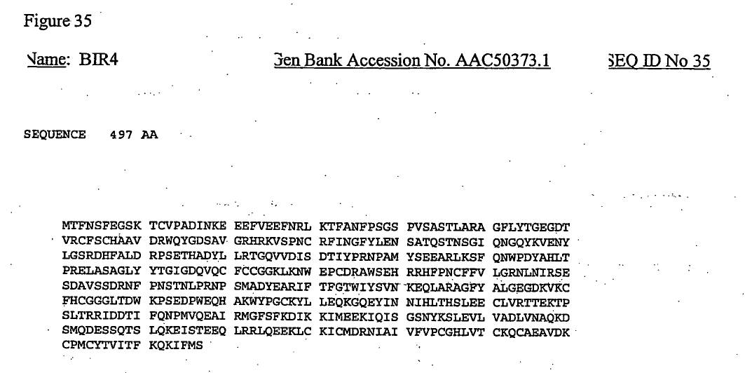

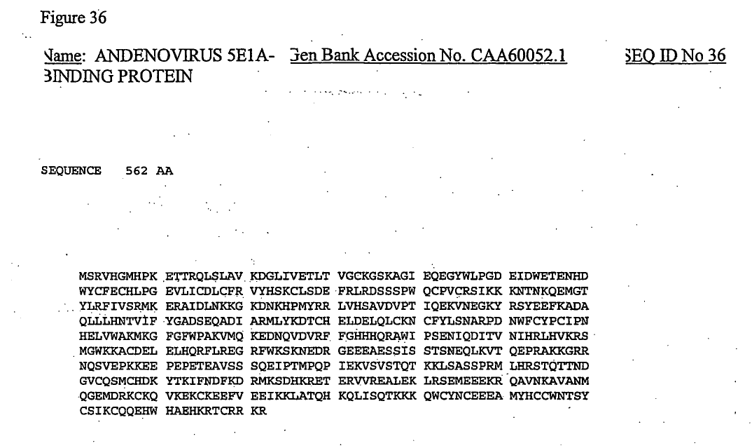

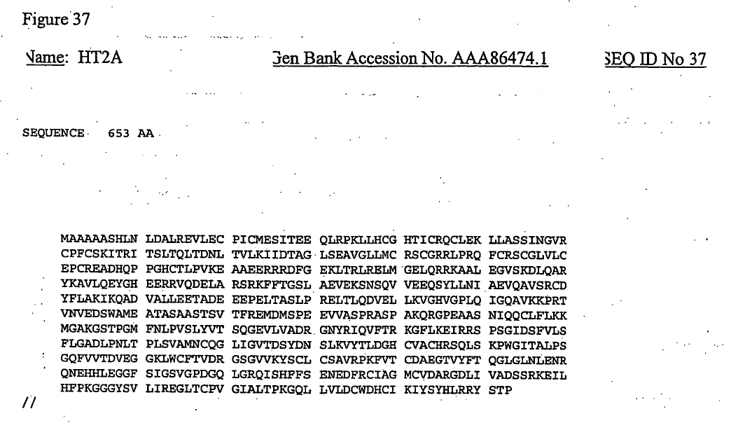

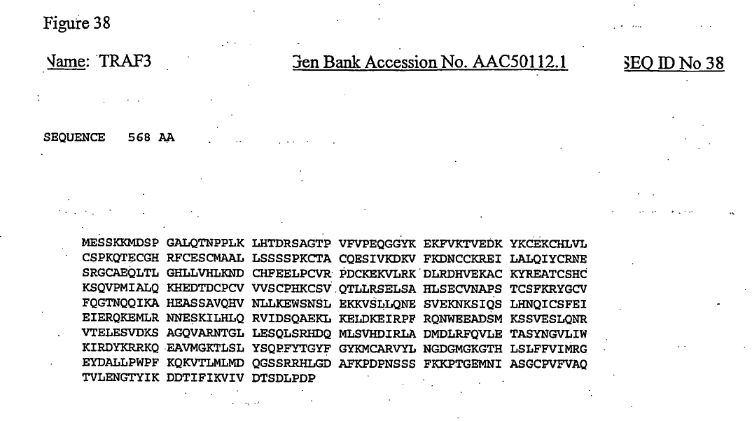

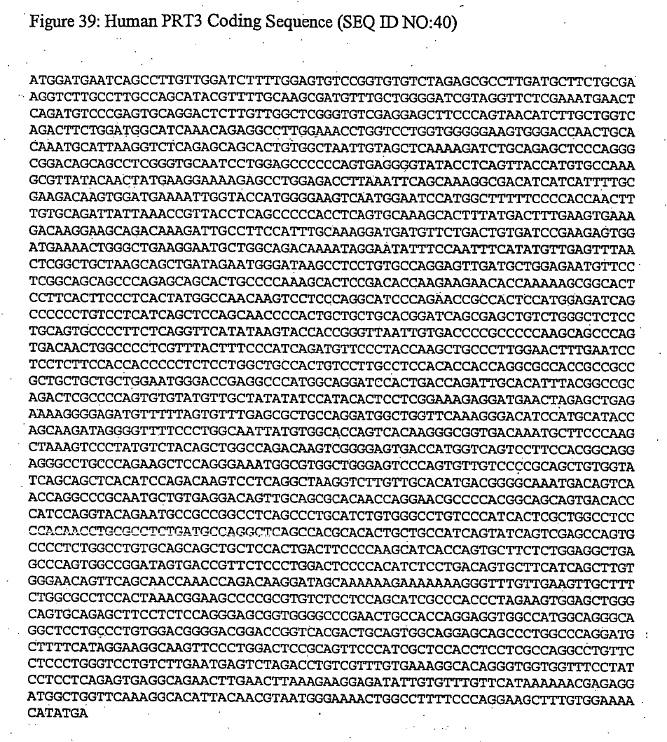

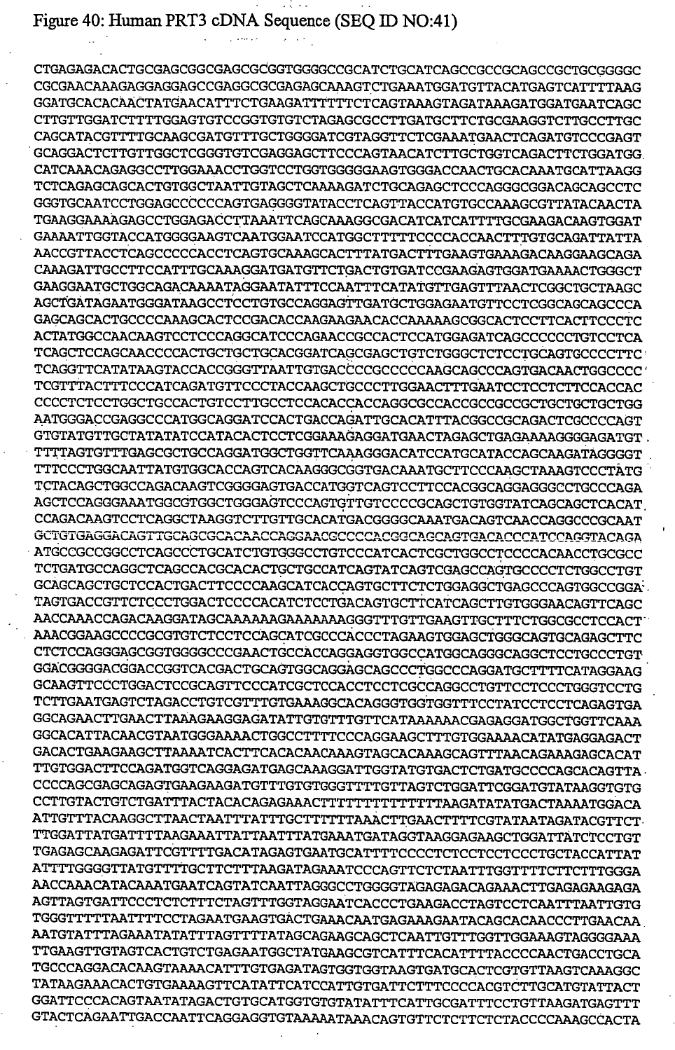

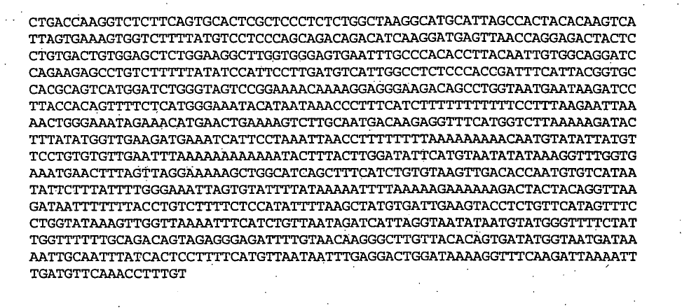

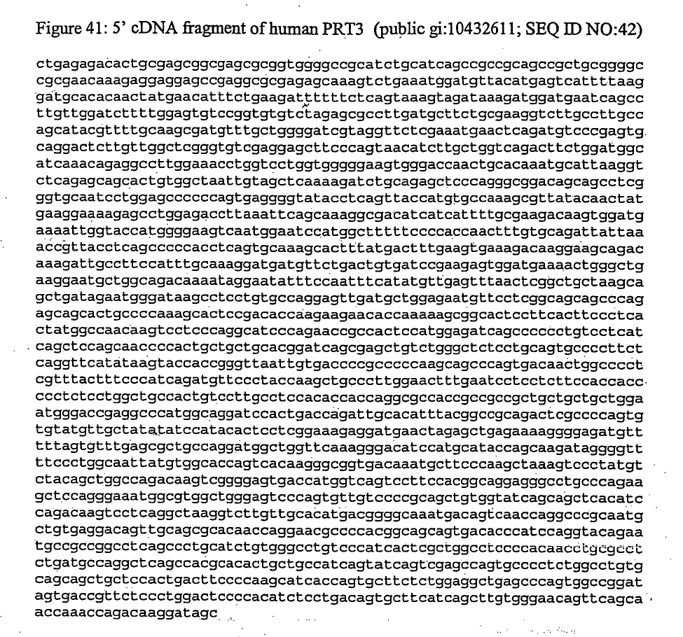

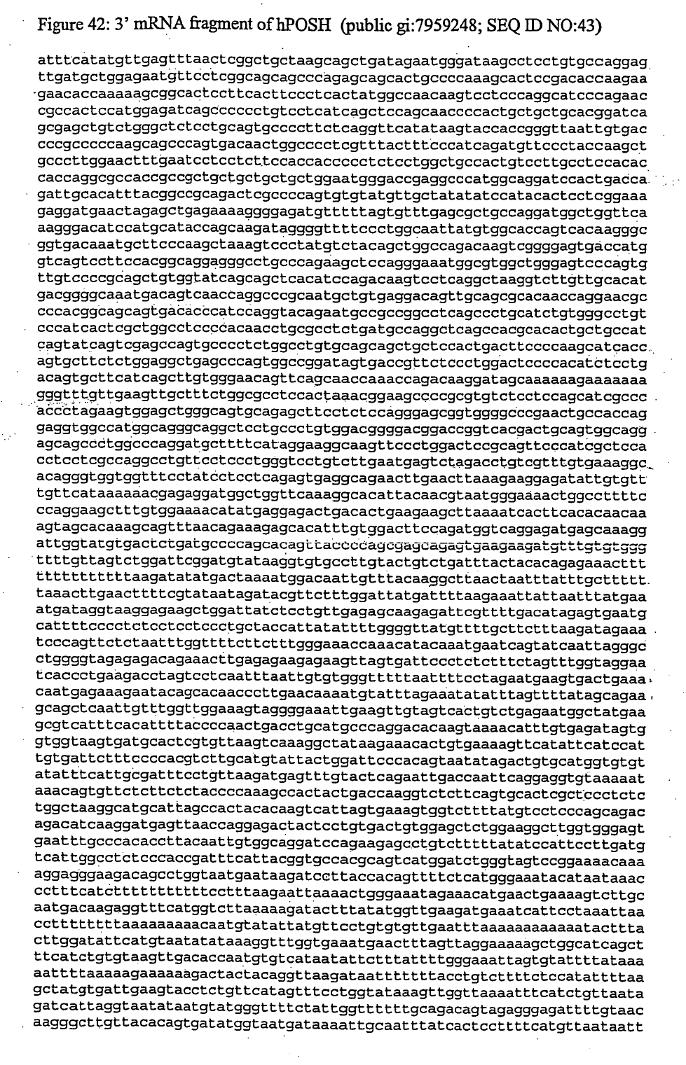

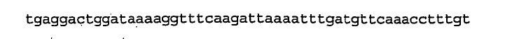

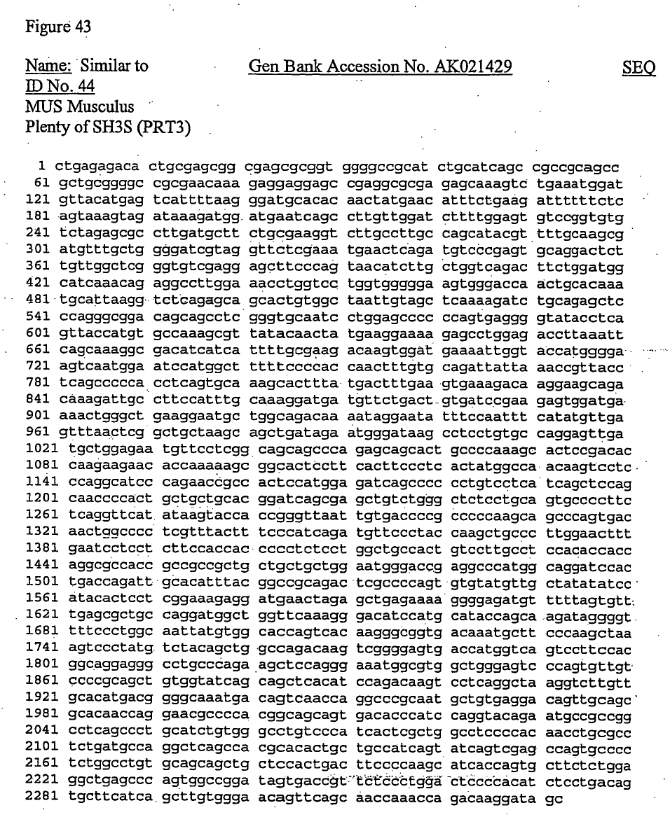

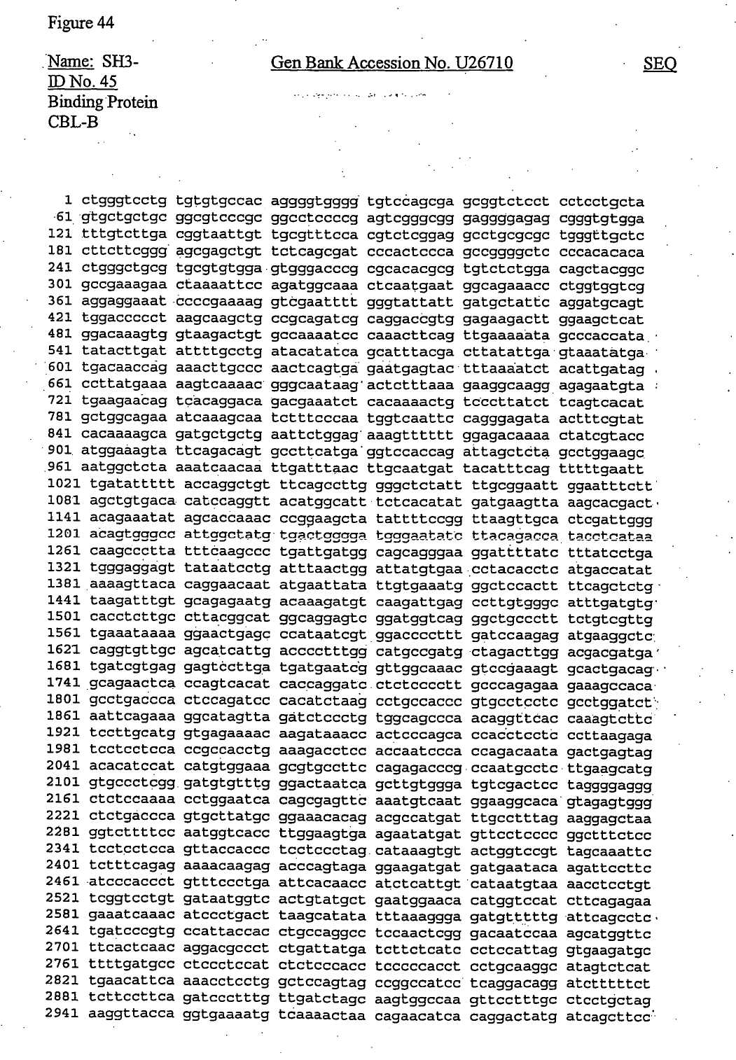

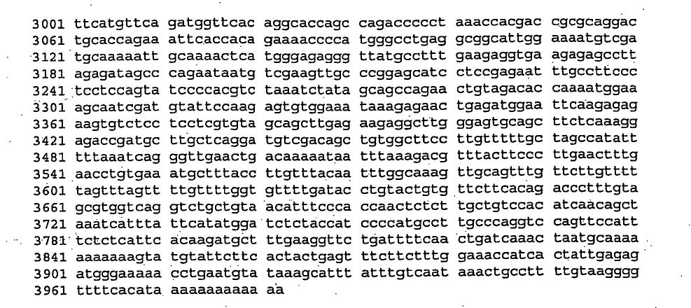

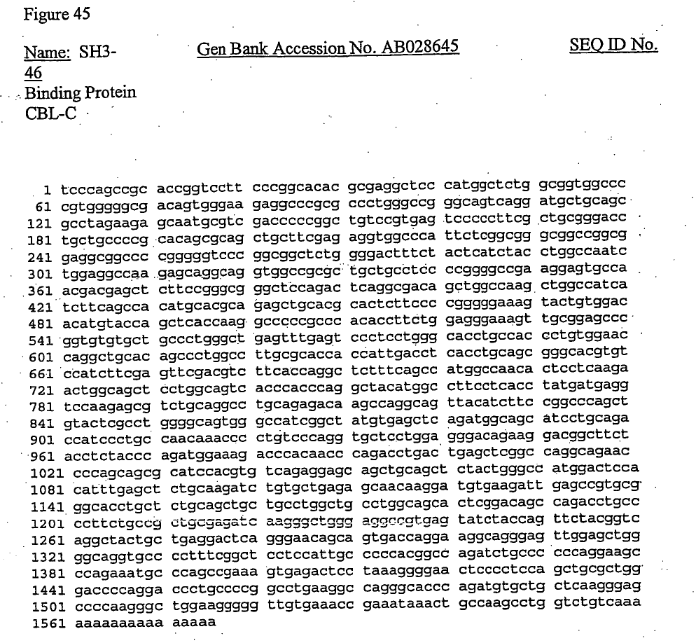

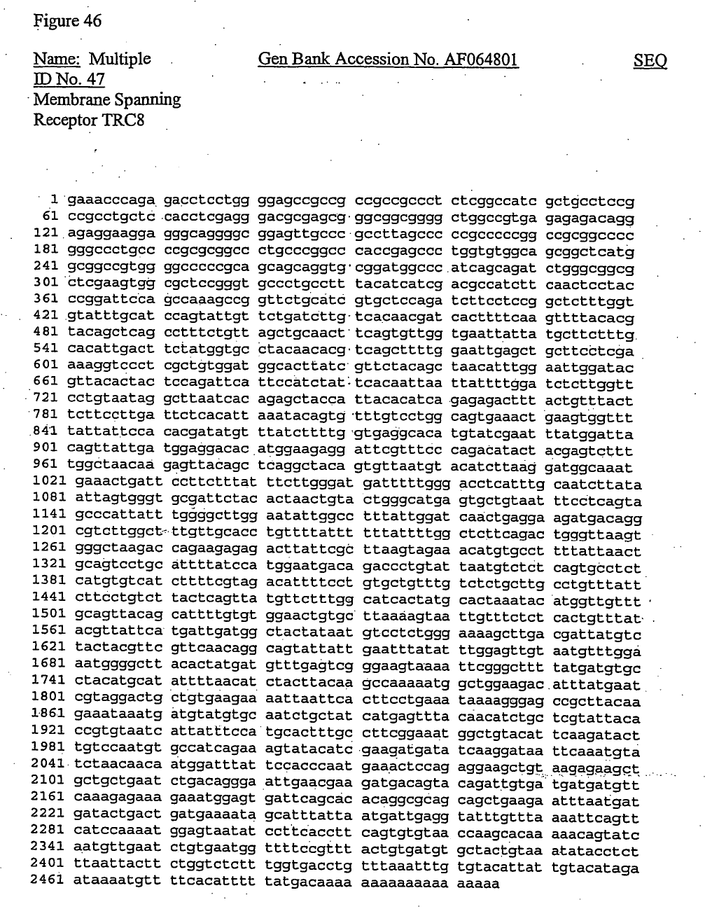

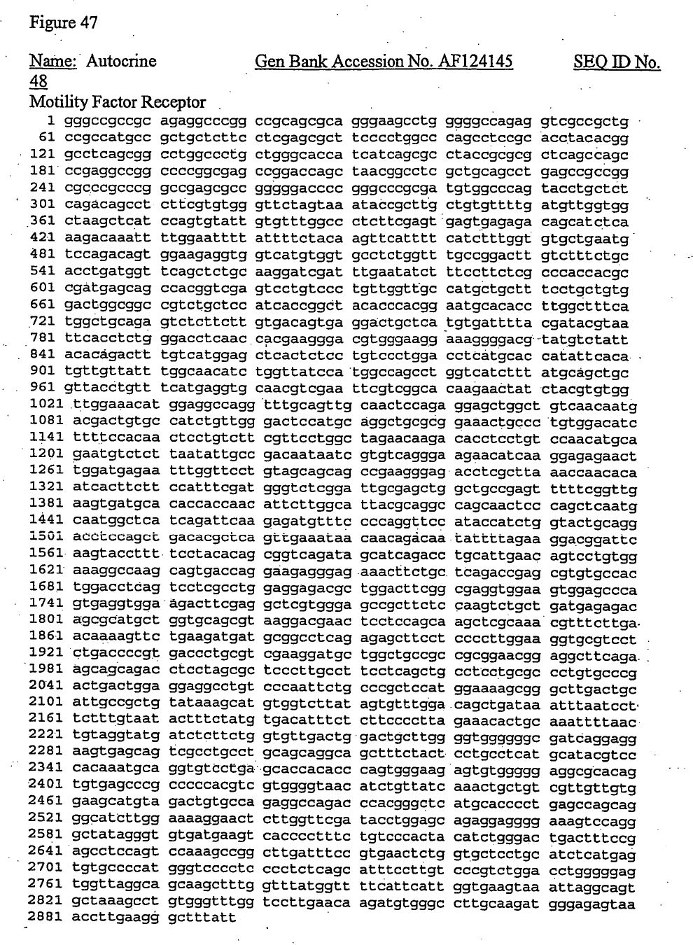

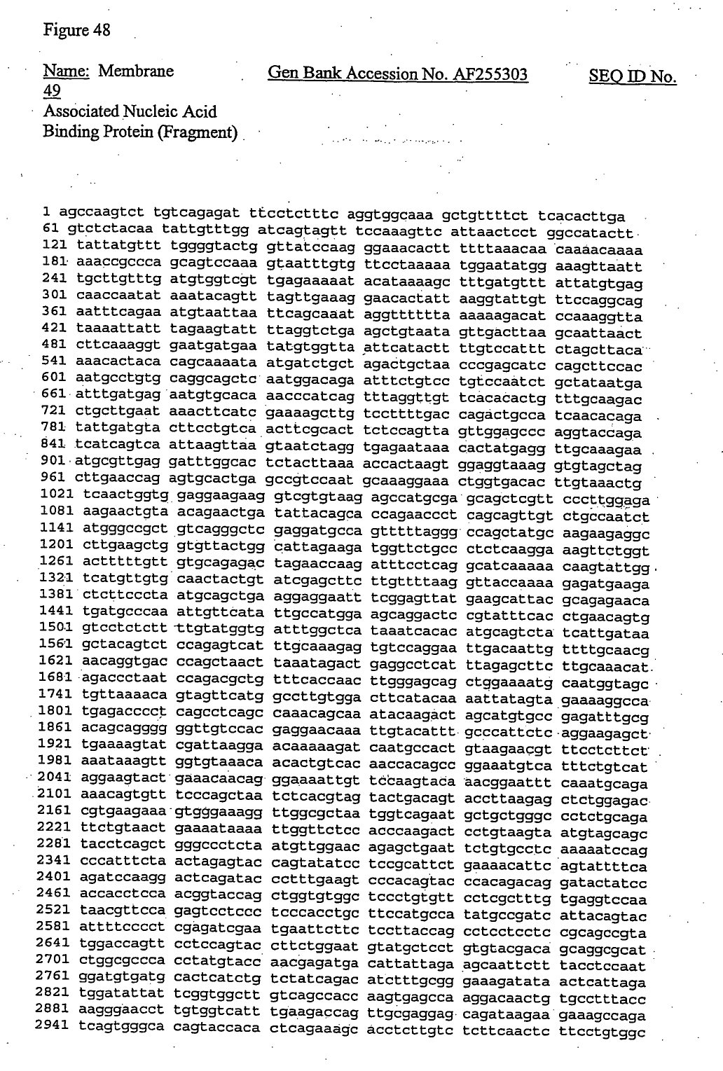

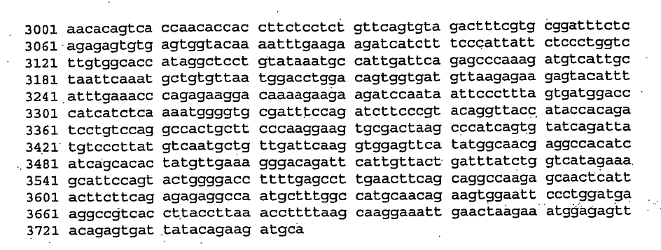

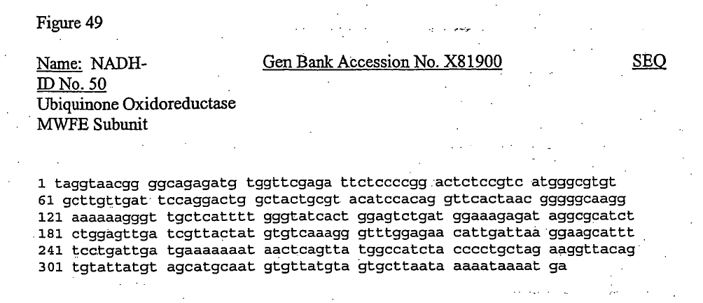

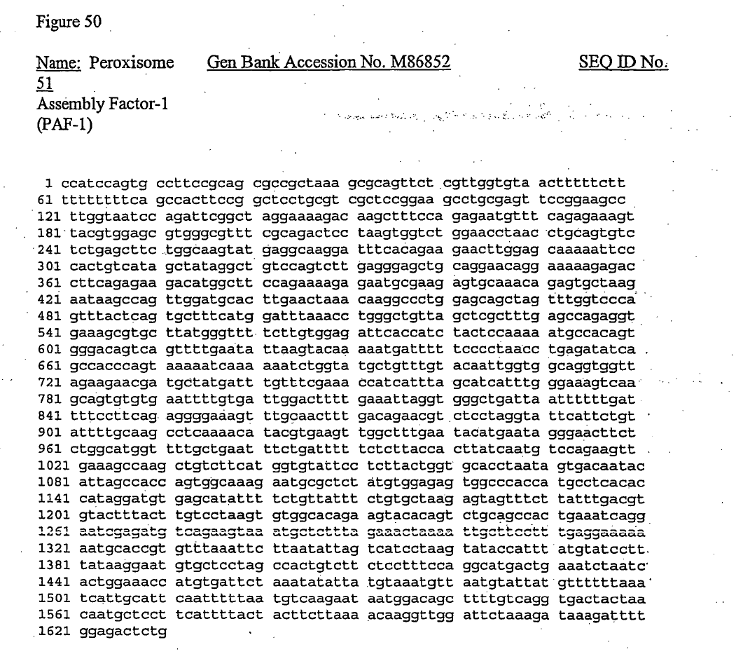

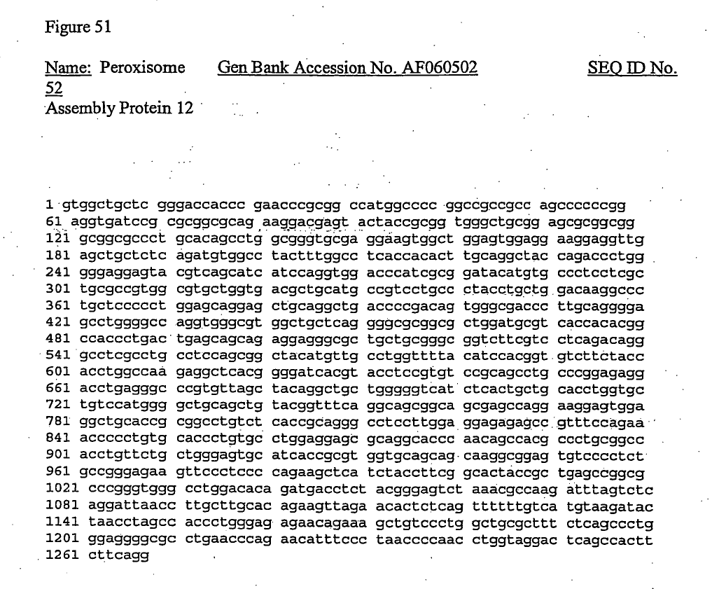

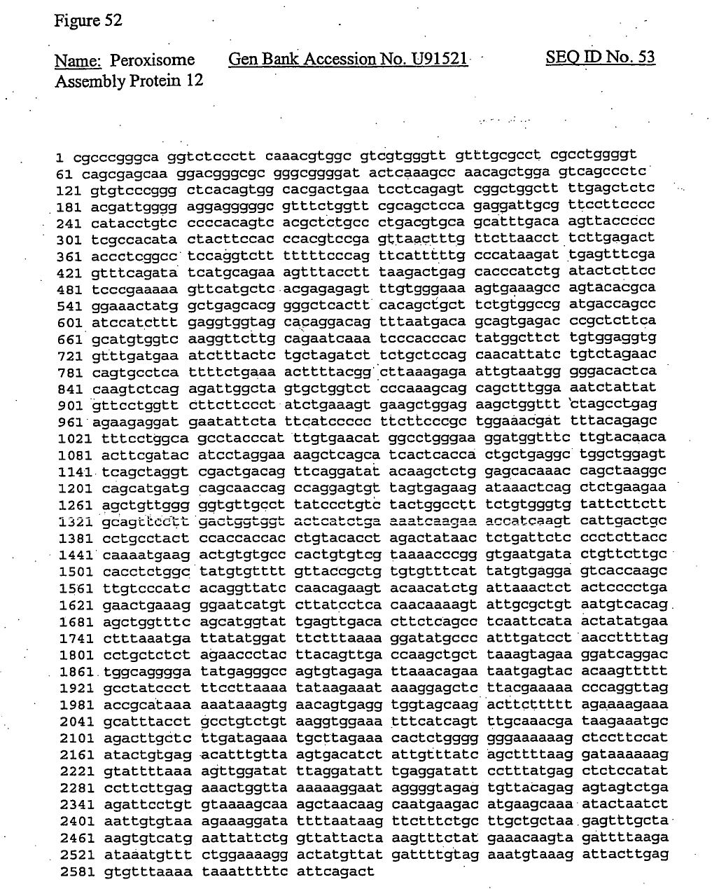

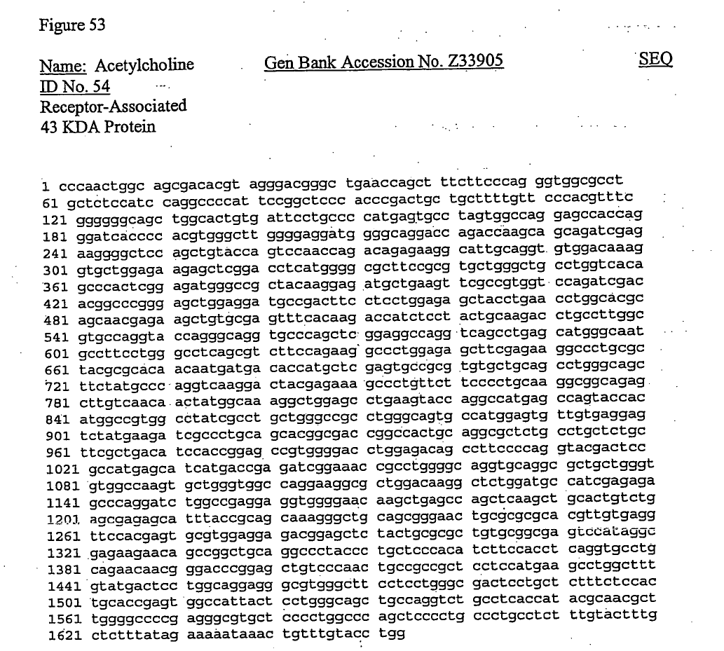

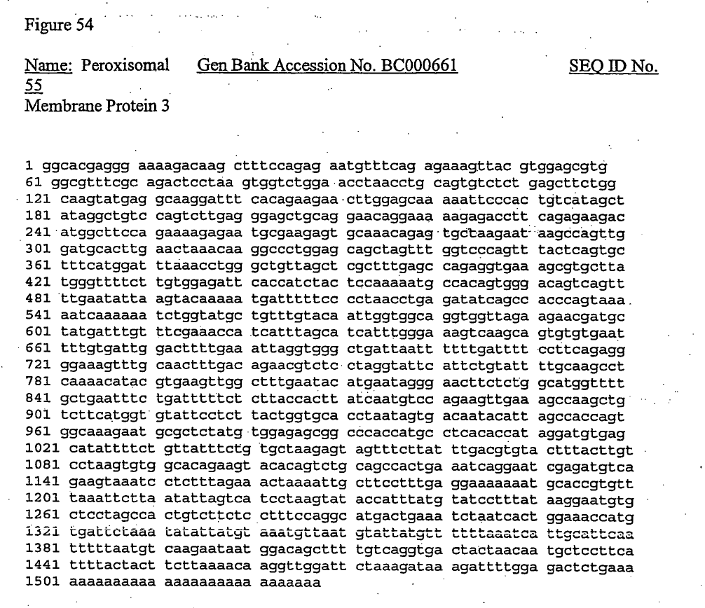

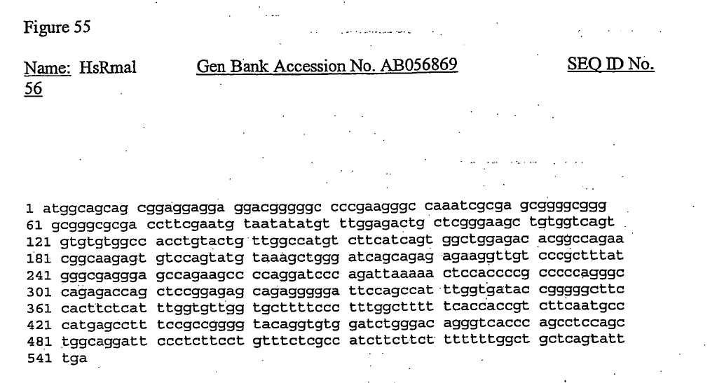

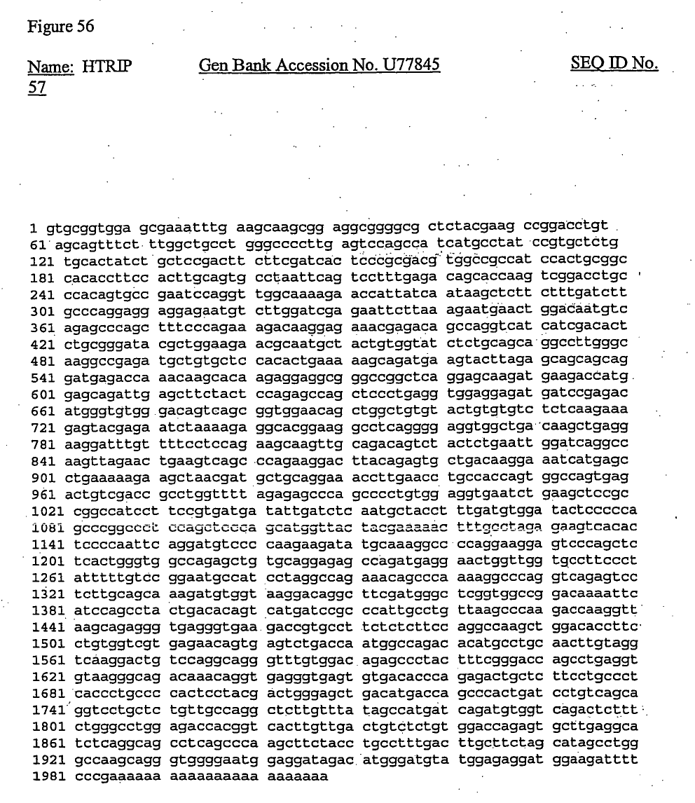

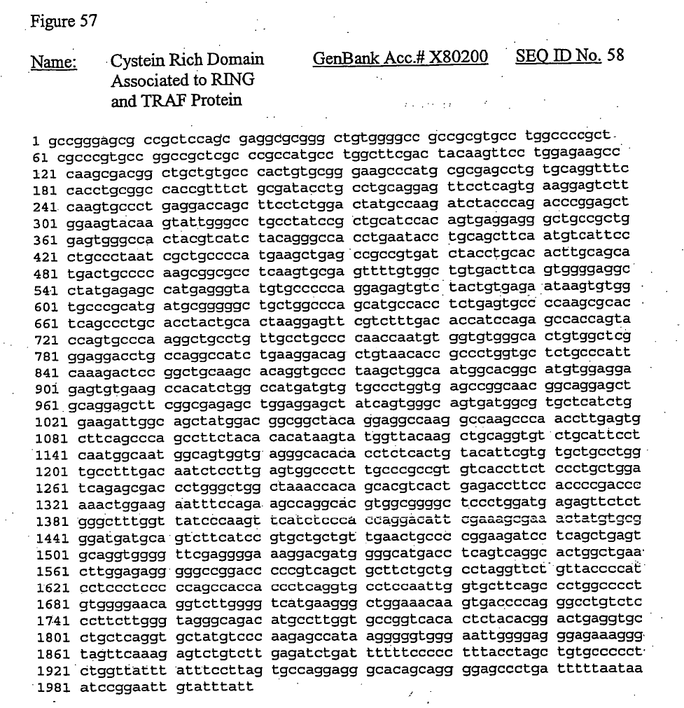

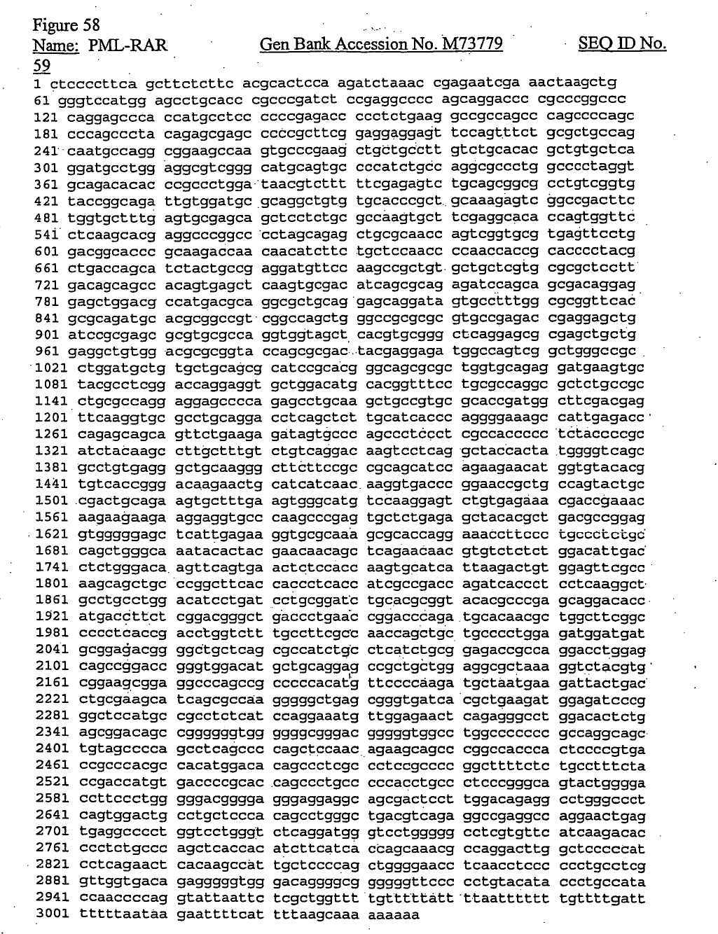

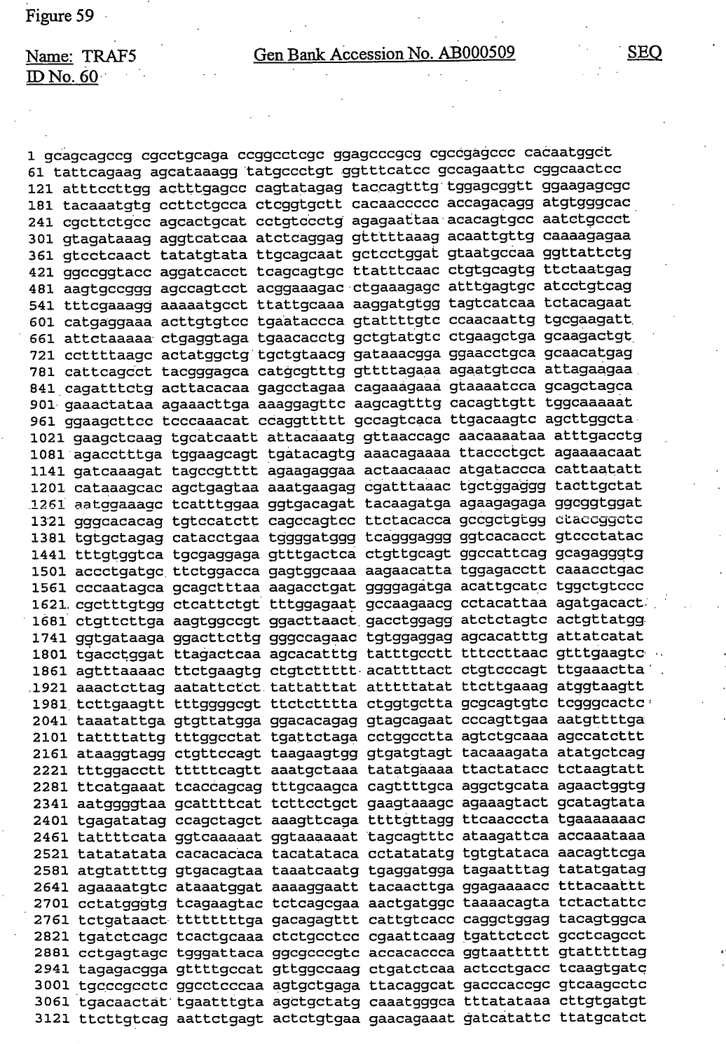

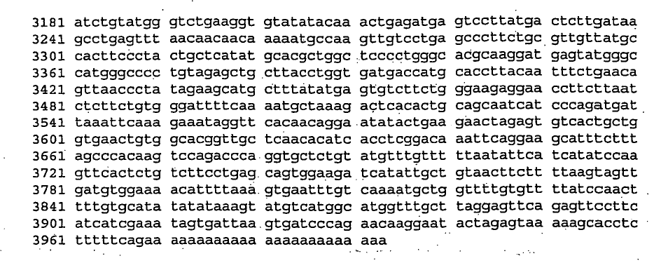

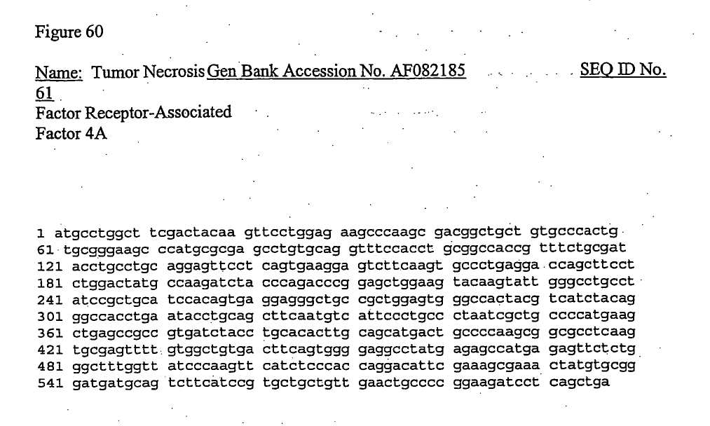

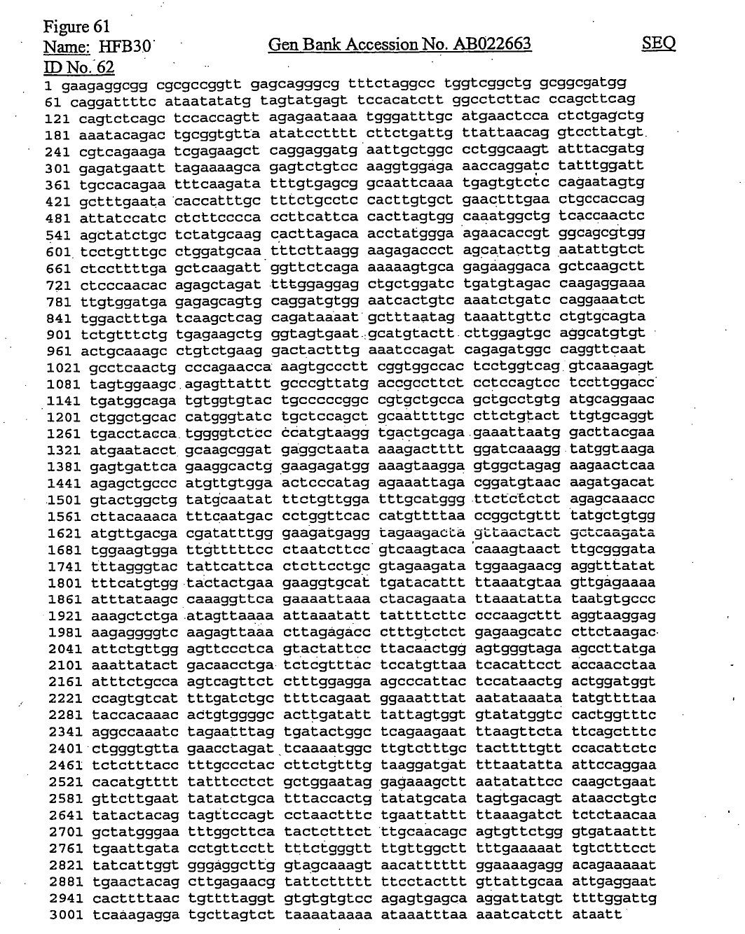

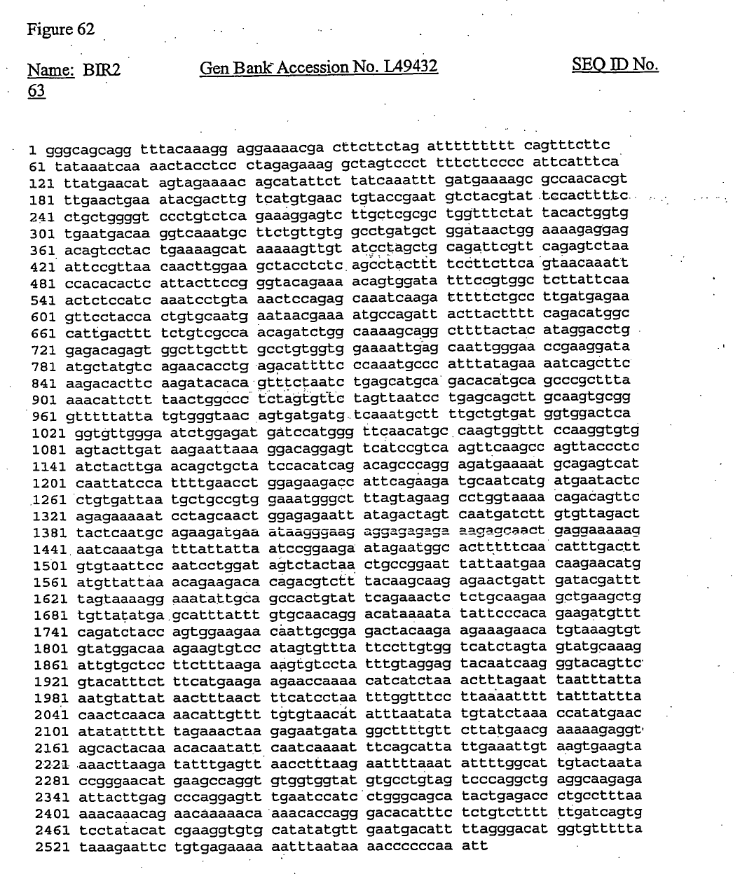

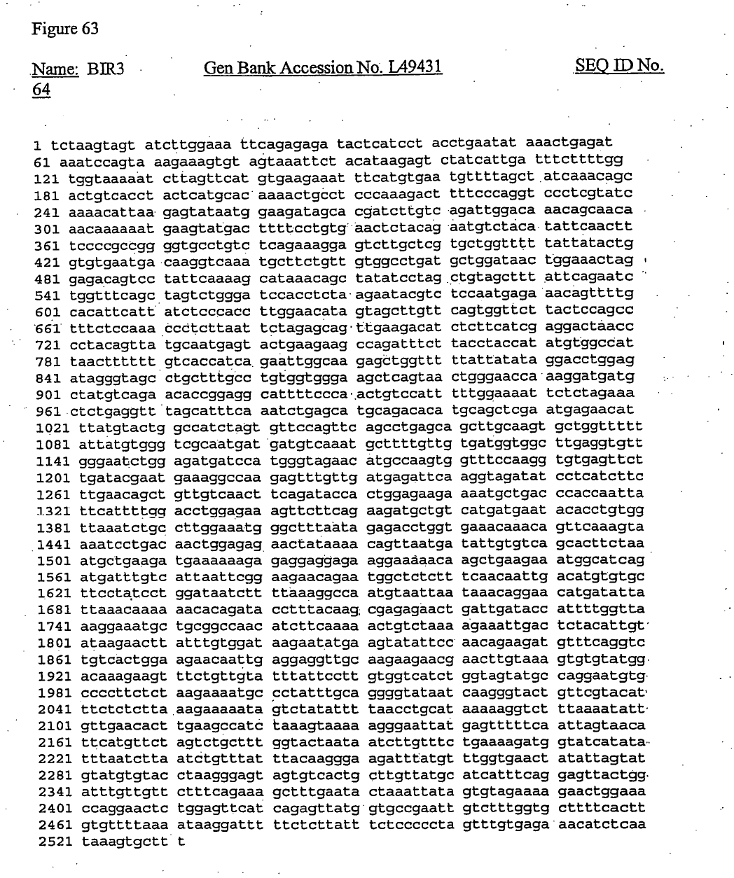

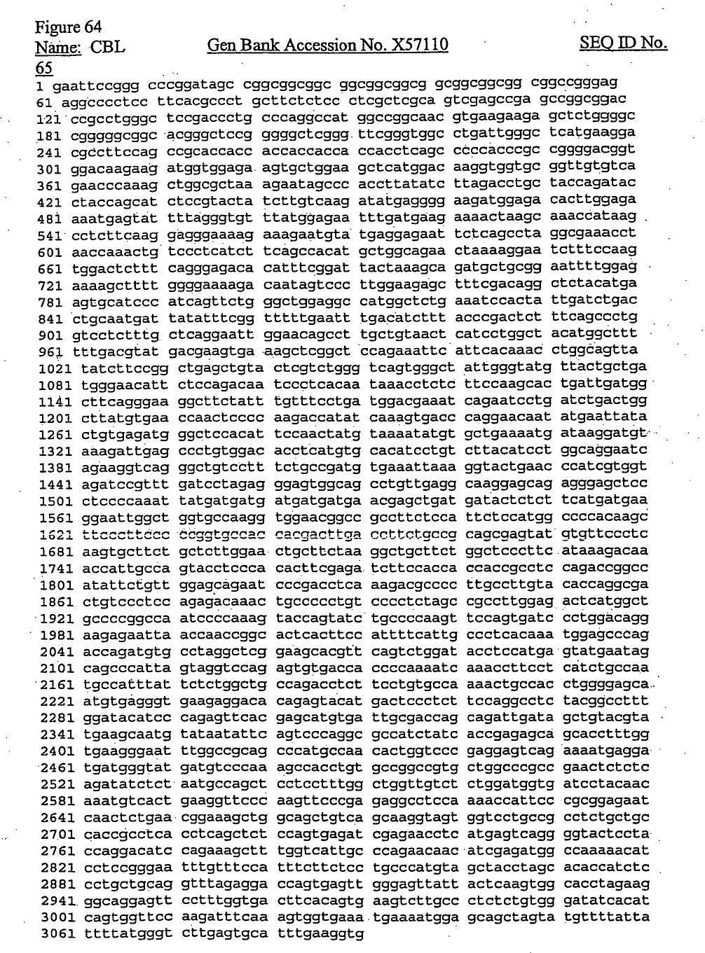

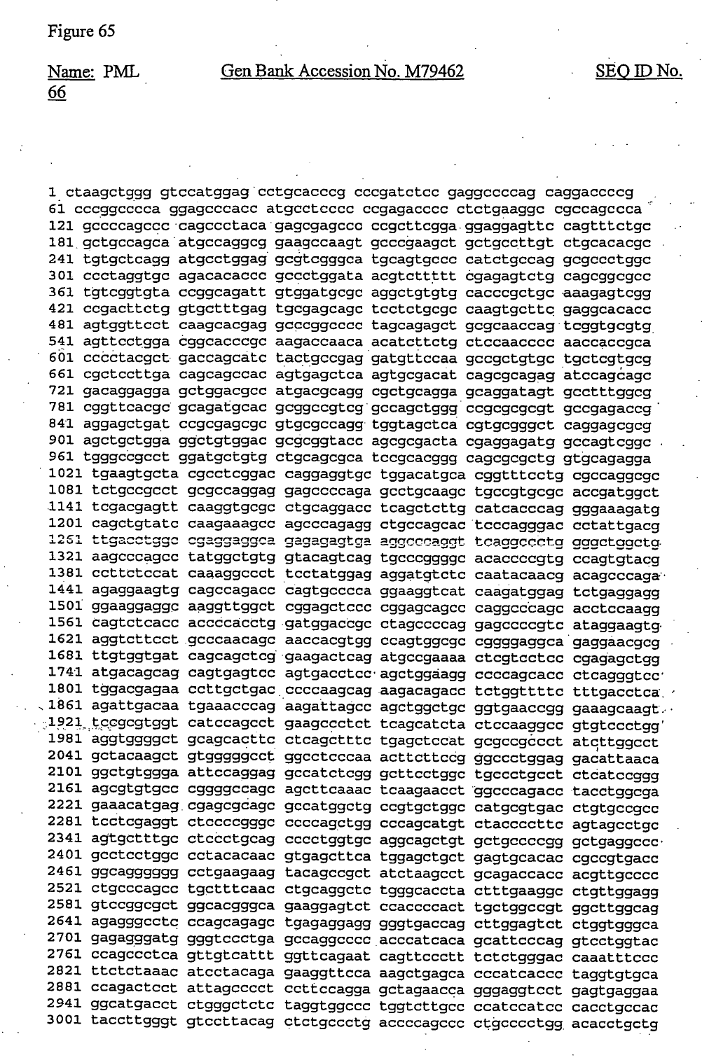

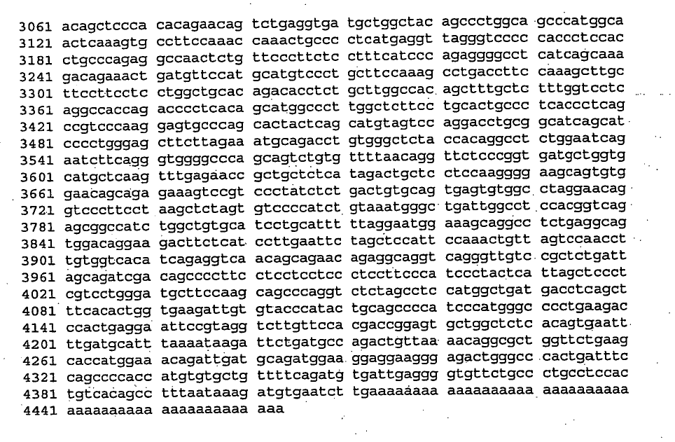

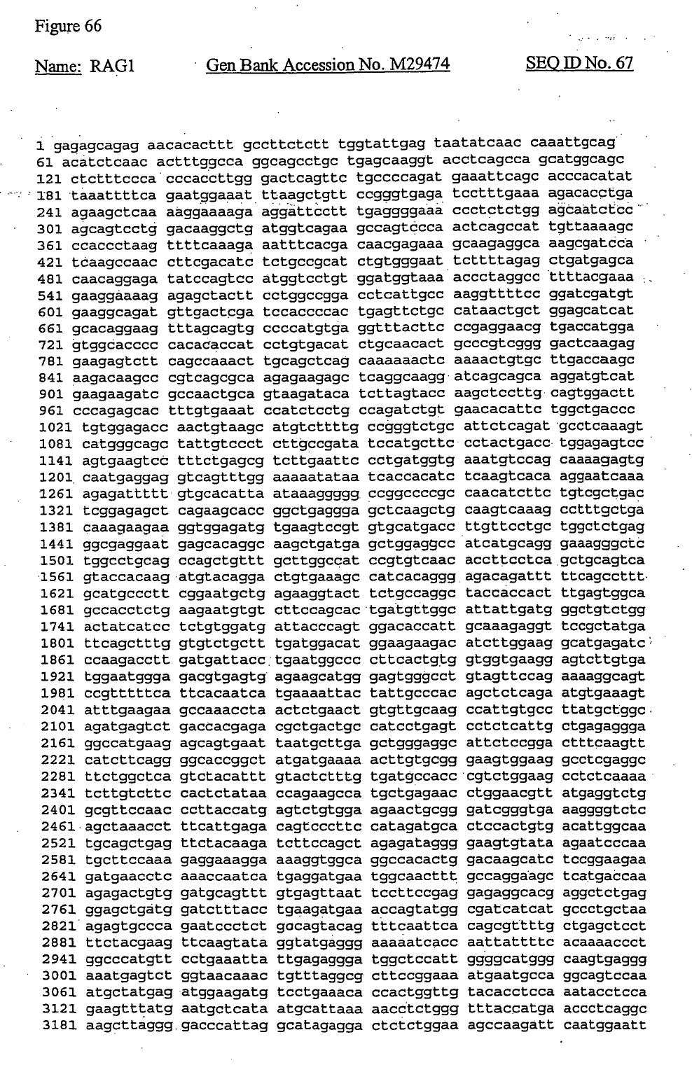

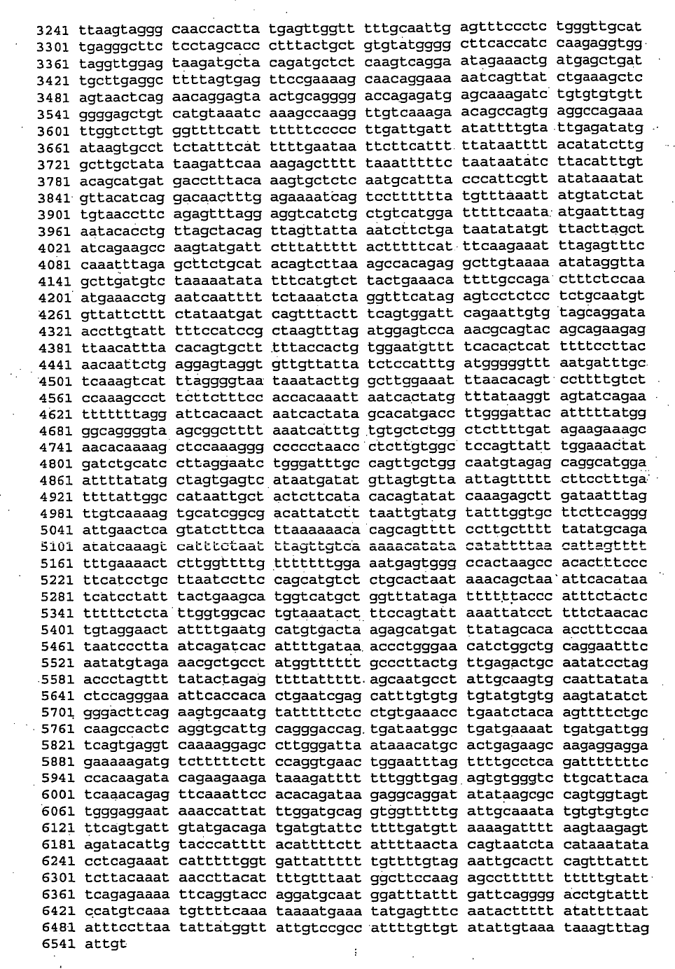

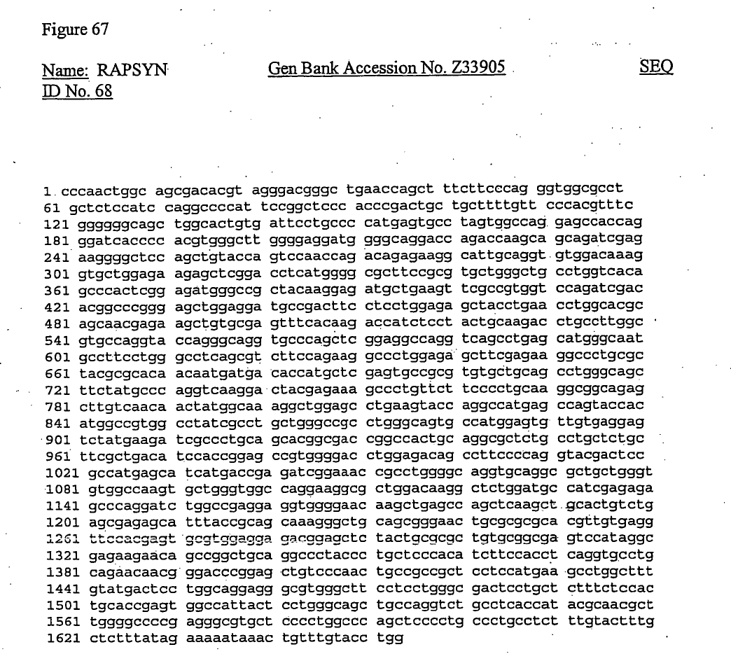

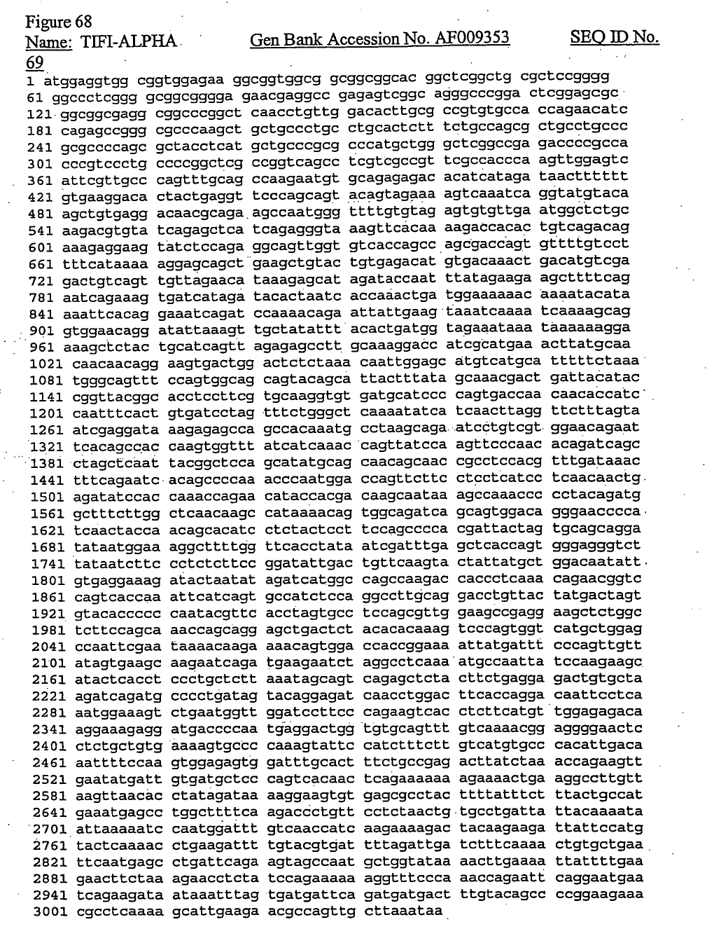

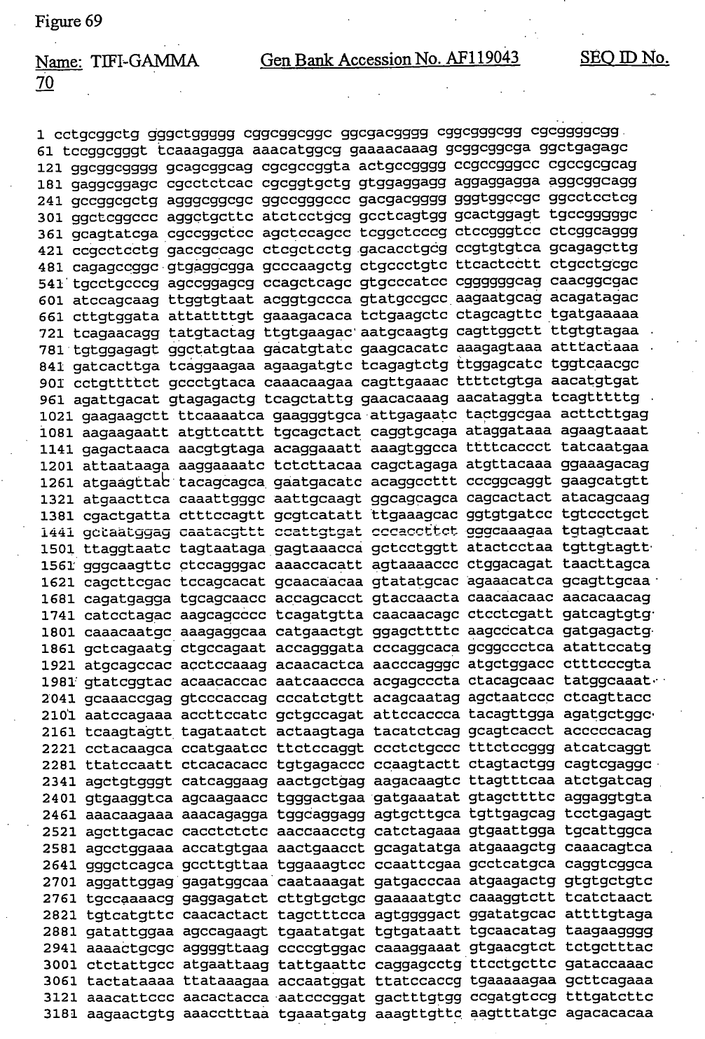

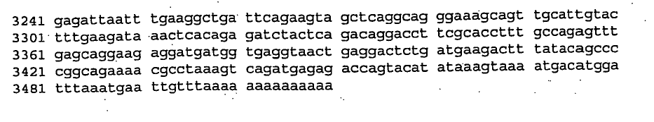

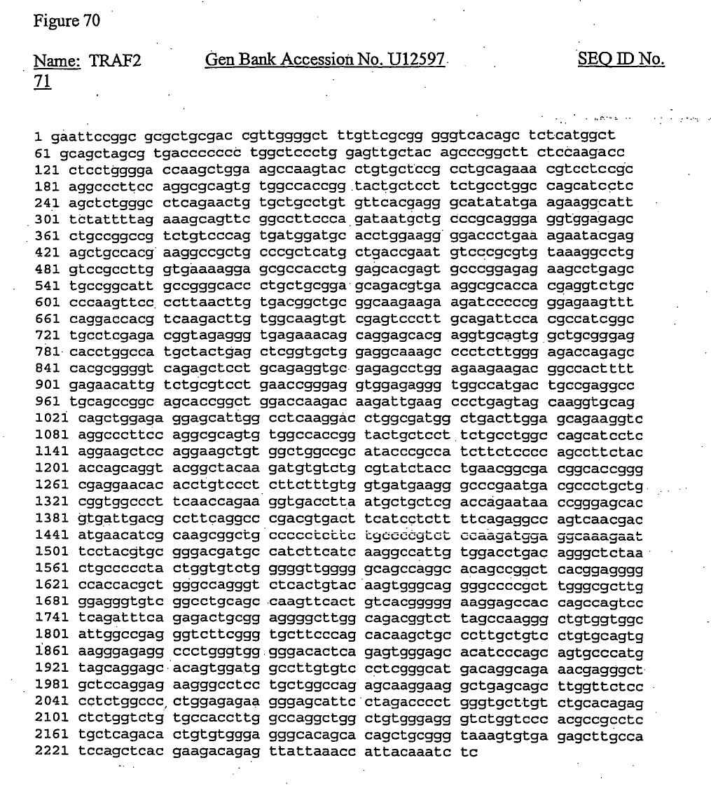

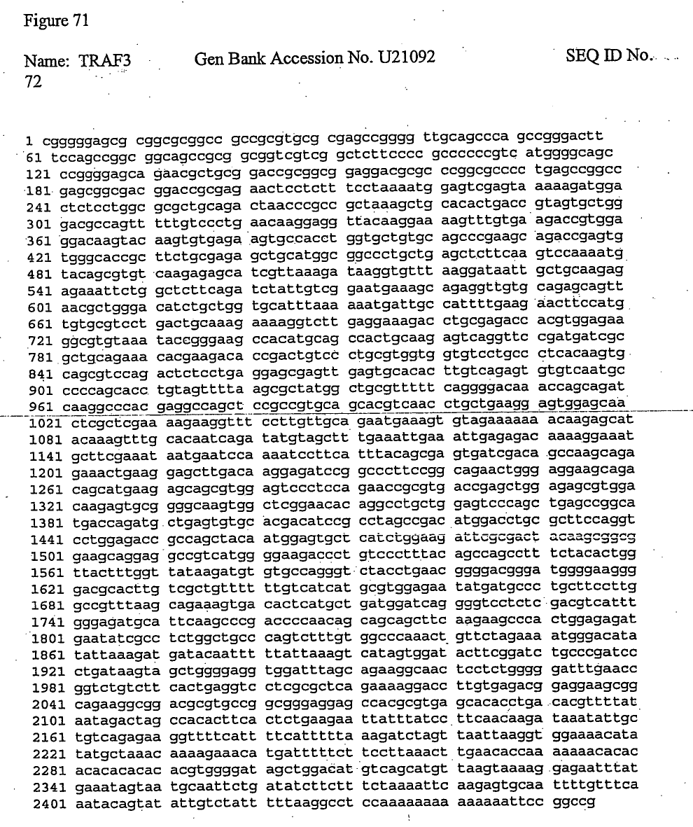

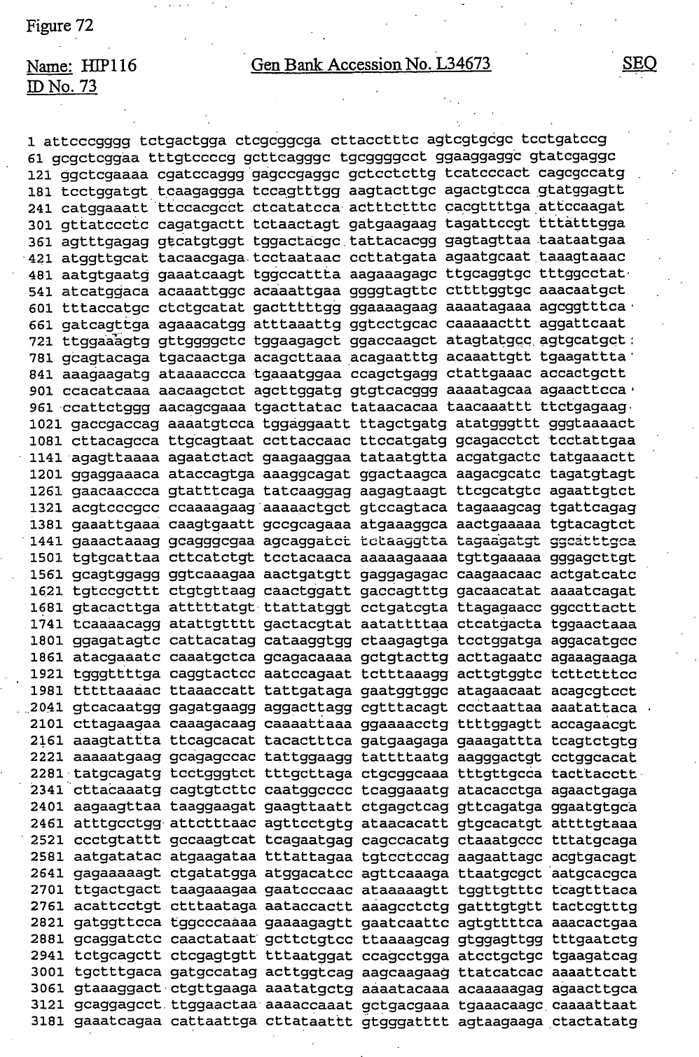

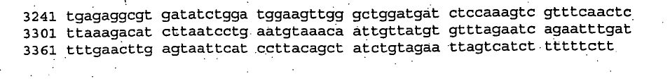

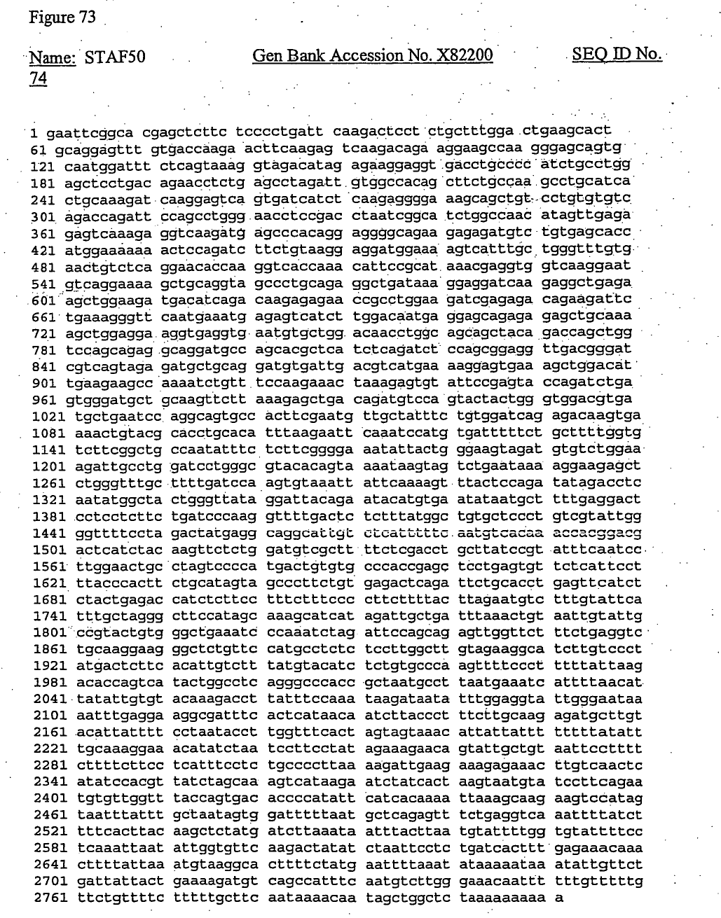

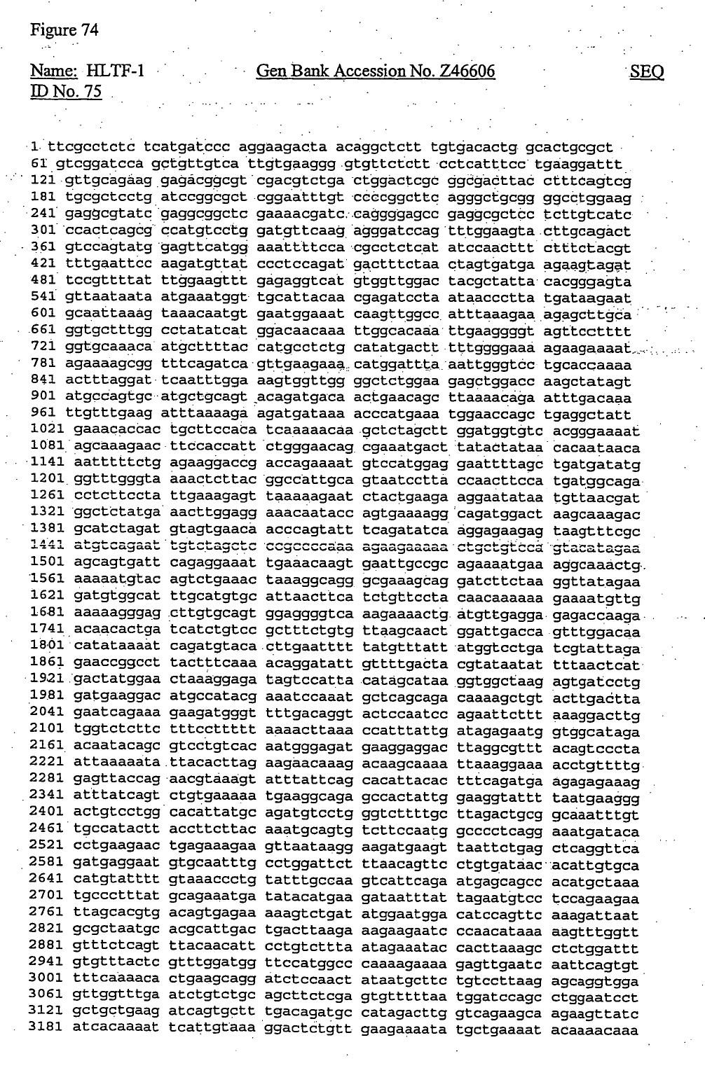

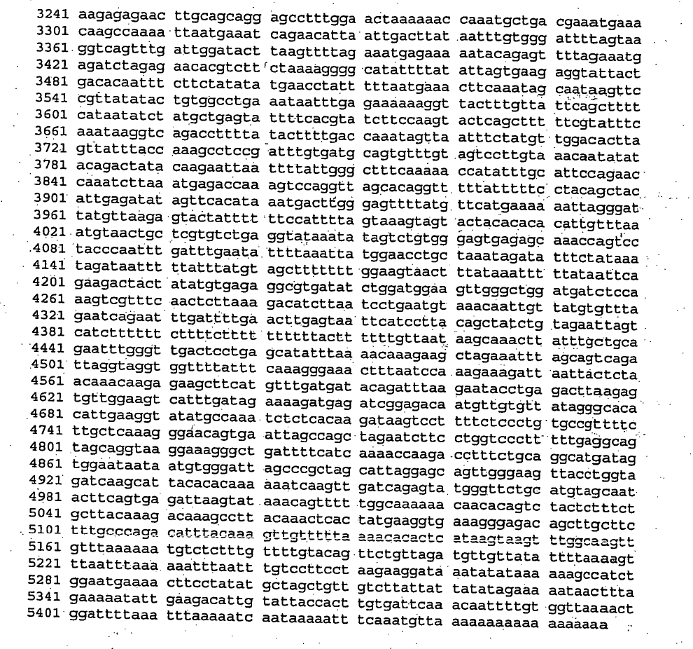

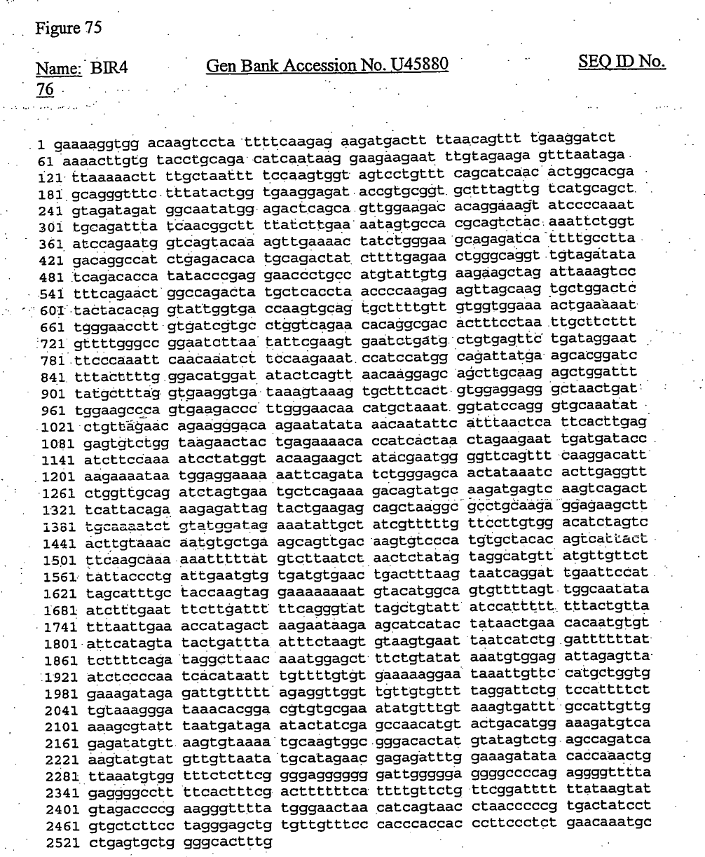

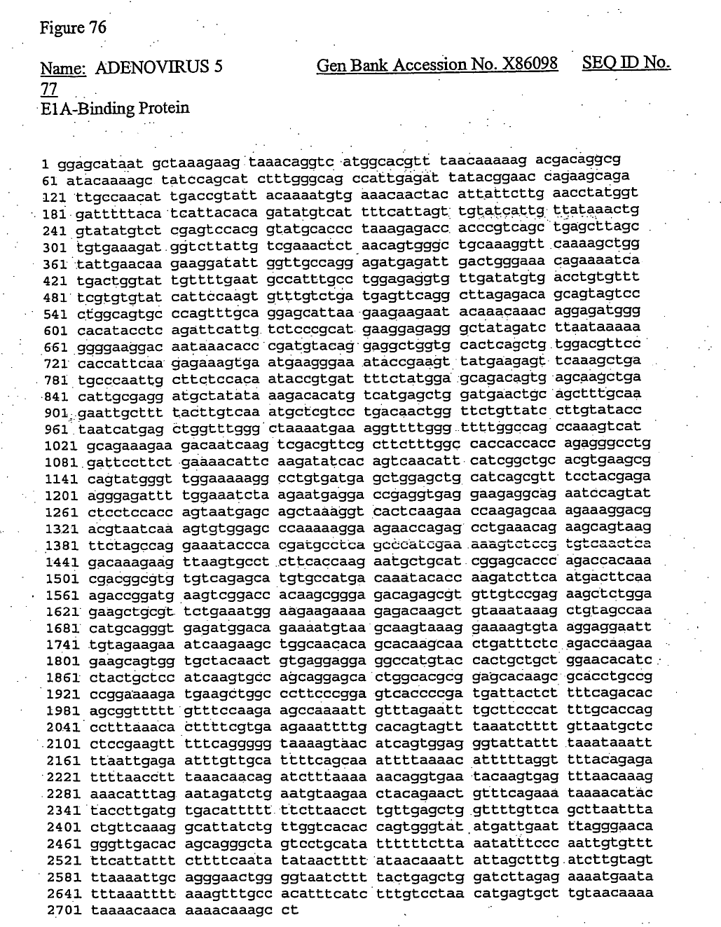

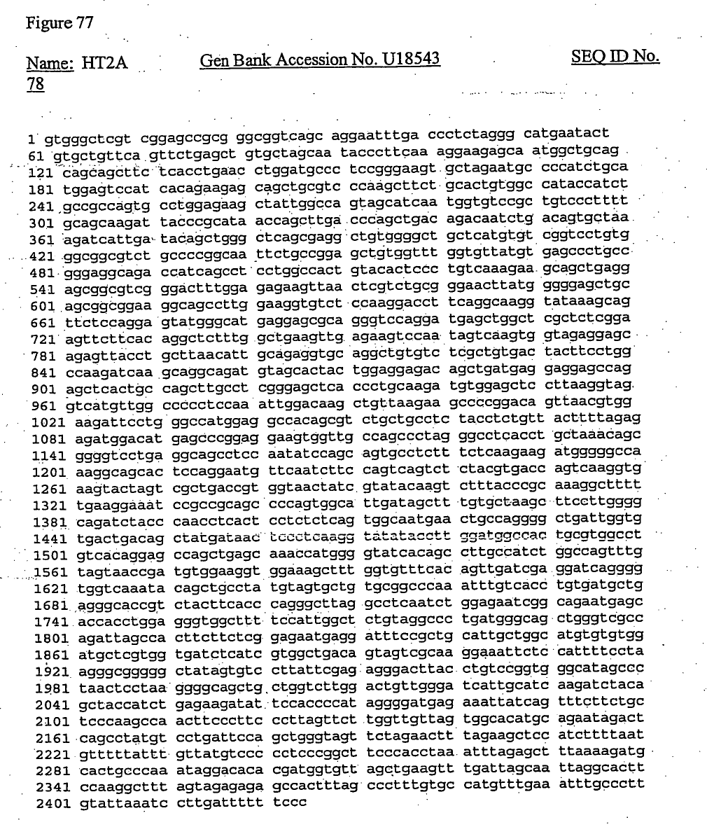

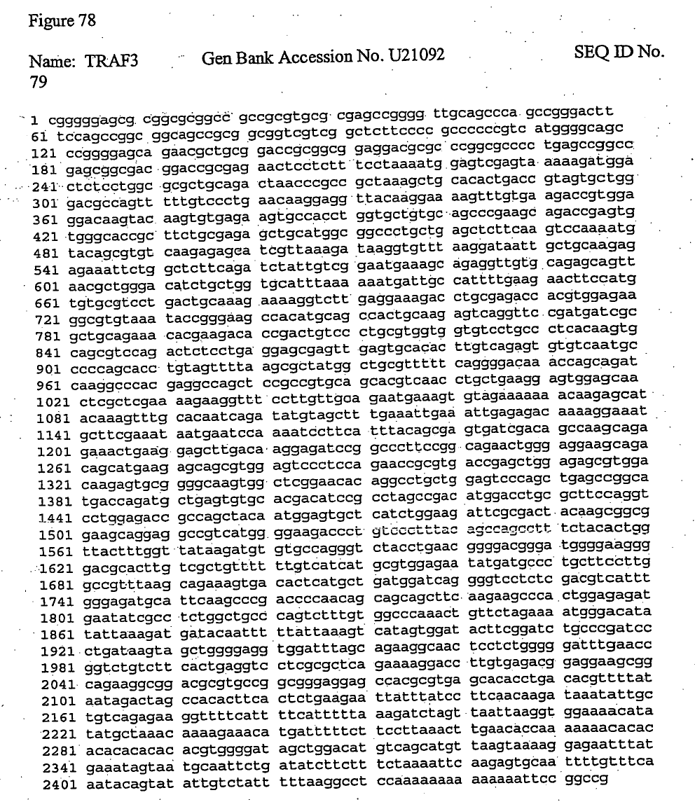

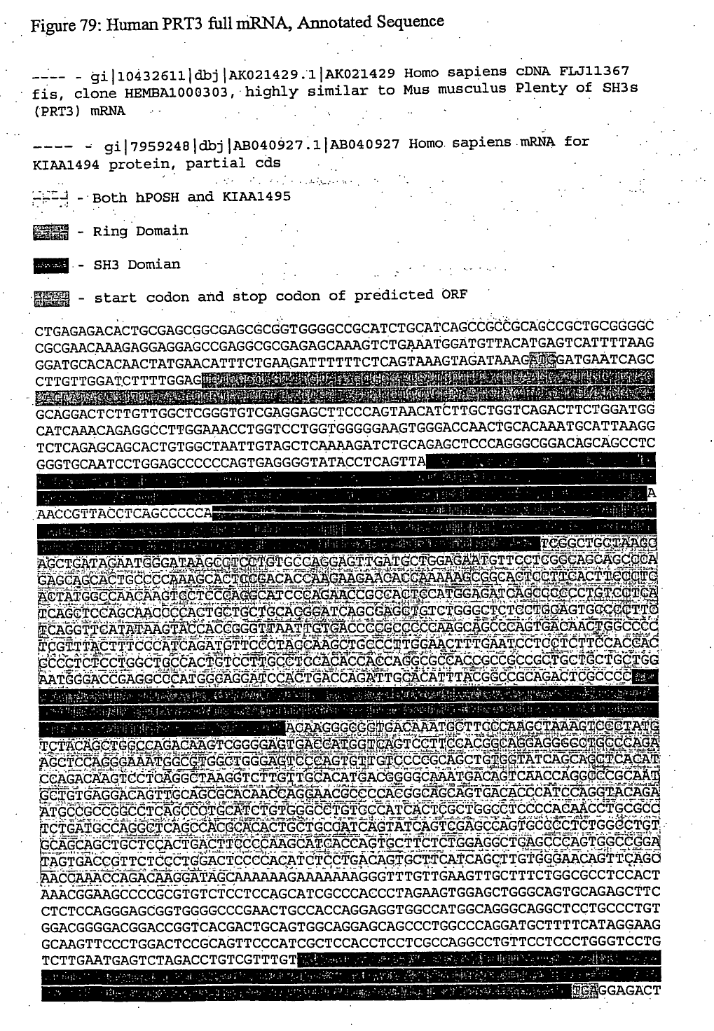

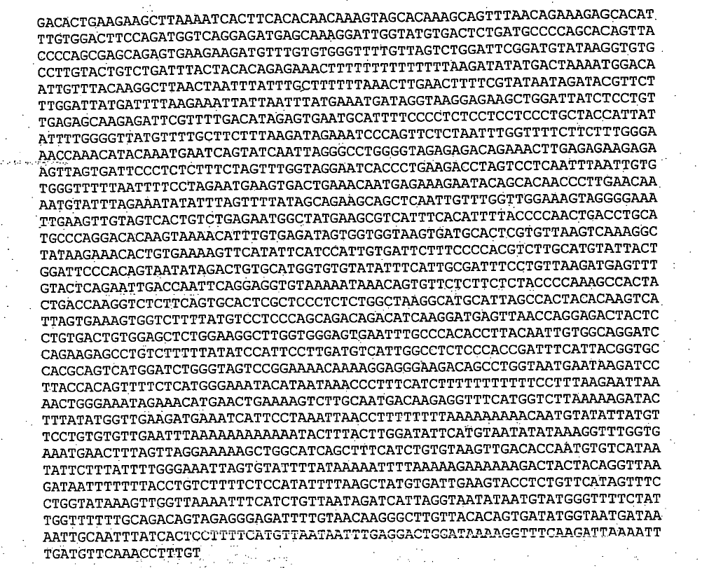

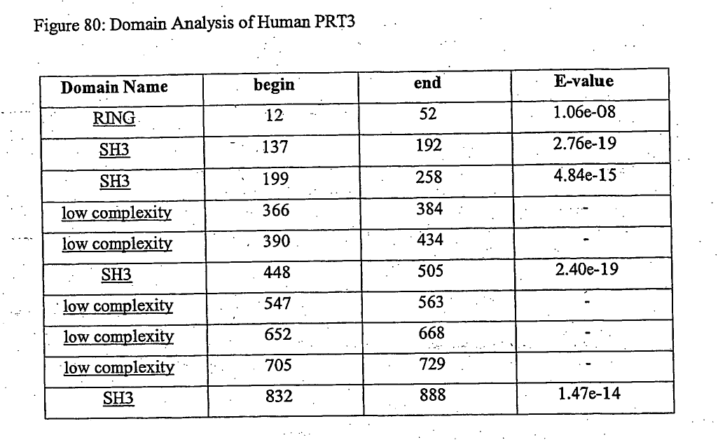

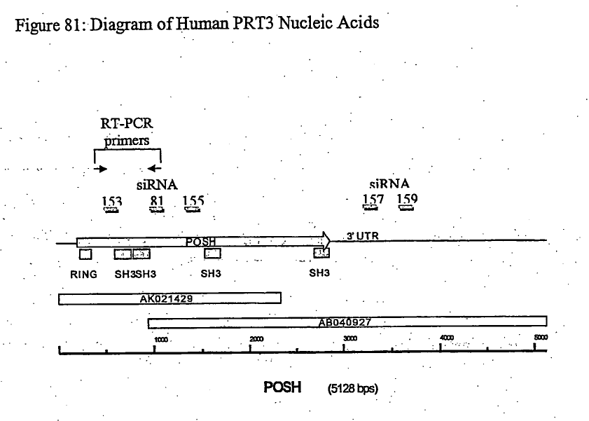

A “C2 domain” is a calcium binding domain. Certain C2 domains comprise the consensus sequence set forth in FIG. 14 as “Consensus/80%”. Other C2 domains comprise the consensus sequences set forth as “Consensus/65%” or “Consensus/50%”.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric protein” or “fusion protein” is a fusion of a first amino acid sequence encoding a polypeptide with a second amino acid sequence defining a domain foreign to and not substantially homologous with any domain of the first amino acid sequence. A chimeric protein may present a foreign domain which is found (albeit in a different protein) in an organism which also expresses the first protein, or it may be an “interspecies”, “intergenic”, etc. fusion of protein structures expressed by different kinds of organisms.

The terms “compound”, “test compound” and “molecule” are used herein interchangeably and are meant to include, but are not limited to, peptides, nucleic acids, carbohydrates, small organic molecules, natural product extract libraries, and any other molecules (including, but not limited to, chemicals, metals and organometallic compounds).

The phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer., Principles of Protein Structure, Springer-Verlag). Examples of amino acid groups defined in this manner include:
(i) a charged group, consisting of Glu and Asp, Lys, Arg and His,
(ii) a positively-charged group, consisting of Lys, Arg and His,
(iii) a negatively-charged group, consisting of Glu and Asp,
(iv) an aromatic group, consisting of Phe, Tyr and Trp,
(v) a nitrogen ring group, consisting of His and Trp,
(vi) a large aliphatic nonpolar group, consisting of Val, Leu and Ile,
(vii) a slightly-polar group, consisting of Met and Cys,
(viii) a small-residue group, consisting of Ser, Thr, Asp, Asn, Gly, Ala, Glu, Gln and Pro,
(ix) an aliphatic group consisting of Val, Leu, Ile, Met and Cys, and
(x) a small hydroxyl group consisting of Ser and Thr.

In addition to the groups presented above, each amino acid residue may form its own group, and the group formed by an individual amino acid may be referred to simply by the one and/or three letter abbreviation for that amino acid commonly used in the art.

A “conserved residue” is an amino acid that is relatively invariant across a range of similar proteins. Often conserved residues will vary only by being replaced with a similar amino acid, as described above for “conservative amino acid substitution”.

The term “domain” as used herein refers to a region of a protein that comprises a particular structure and/or performs a particular function.

The term “Gag protein” or “Gag polypeptide” refers to a polypeptide having Gag activity and preferably comprising an L (or late) domain. Exemplary Gag proteins include a motif such as PXXP, PPXY, PXXY, YXXL, RXXPXXP, RPDPTAP, RPLPVAP, RPEPTAP, PTAPPEY, PTAPPEE and/or RPEPTAPPEE. An exemplary HIV-1 Gag protein is SEQ ID NO: 32. Typically, an HIV Gag protein comprises a p6 protein.

“Homology” or “identity” or “similarity” refers to sequence similarity between two peptides or between two nucleic acid molecules. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. A sequence which is “unrelated” or “non-homologous” shares less than 40% identity, though preferably less than 25% identity with a sequence of the present invention. In comparing two sequences, the absence of residues (amino acids or nucleic acids) or presence of extra residues also decreases the identity and homology/similarity.

The term “homology” describes a mathematically based comparison of sequence similarities which is used to identify genes or proteins with similar functions or motifs. The nucleic acid and protein sequences of the present invention may be used as a “query sequence” to perform a search against public databases to, for example, identify other family members, related sequences or homologs. Such searches can be performed using the NBLAST and XBLAST programs (version 2.0) of Altschul, et al. (1990) J. Mol. Biol. 215:403-10. BLAST nucleotide searches can be performed with the NBLAST program, score—100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used. See http://www.ncbi.nlm.nih.gov.

As used herein, “identity” means the percentage of identical nucleotide or amino acid residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

The term “including” is used herein to mean, and is used interchangeably with, the phrase “including but not limited to”.

The term “intron” refers to a portion of nucleic acid that is intially transcribed into RNA but later removed such that it is not, for the most part, represented in the processed mRNA. Intron removal occurs through reactions at the 5′ and 3′ ends, typically referred to as 5′ and 3′ splice sites, respectively. Alternate use of different splice sites results in splice variants. An intron is not necessarily situated between two “exons”, or portions that code for amino acids, but may instead be positioned, for example, between the promoter and the first exon. An intron may be self-splicing or may require cellular components to be spliced out of the mRNA. A “heterologous intron” is an intron that is inserted into a coding sequence that is not naturally associated with that coding sequence. In addition, a heterologous intron may be a generally natural intron wherein one or both of the splice sites have been altered to provide a desired quality, such as increased or descreased splice efficiency. Heterologous introns are often inserted, for example, to improve expression of a gene in a heterologous host, or to increase the production of one splice variant relative to another. As an example, the rabbit beta-globin gene may be used, and is commercially available on the pCI vector from Promega Inc. Other exemplary introns are provided in Lacy-Hulbert et al. (2001) Gene Ther 8(8):649-53.

The term “isolated”, as used herein with reference to the subject proteins and protein complexes, refers to a preparation of protein or protein complex that is essentially free from contaminating proteins that normally would be present with the protein or complex, e.g., in the cellular milieu in which the protein or complex is found endogenously. Thus, an isolated protein complex is isolated from cellular components that normally would “contaminate” or interfere with the study of the complex in isolation, for instance while screening for modulators thereof. It is to be understood, however, that such an “isolated” complex may incorporate other proteins the modulation of which, by the subject protein or protein complex, is being investigated.

The term “isolated” as also used herein with respect to nucleic acids, such as DNA or RNA, refers to molecules in a form which does not occur in nature. Moreover, an “isolated nucleic acid” is meant to include nucleic acid fragments which are not naturally occurring as fragments and would not be found in the natural state.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The term “maturation” as used herein refers to the processing of viral proteins leading to the pinching off of nascent virion from the cell membrane, including, for example, assembly, budding and release.

A “membrane associated protein” is meant to include proteins that are integral membrane proteins as well as proteins that are stably associated with a membrane.

A “membrane spanning domain” or “transmembrane domain” or membrane domain” is a domain that traverses a biological lipid bilayer membrane from one side to the other. Generally membrane domains are identified as a region of a protein having a high hydrophobicity. Membrane domains are typically between 15 and 25 amino acids in length. Exemplary methods for identifying membrane domains are provided in FIG. 129.

The term “or” is used herein to mean, and is used interchangeably with, the term “and/or”, unless context clearly indicates otherwise.

The term “p6” or p6gag” is used herein to refer to an HIV protein comprising a viral L domain. Antibodies that bind to a p6 domain are referred to as “anti-p6 antibodies”. p6 also refers to proteins that comprise artificially engineered L domains including, for example, L domains comprising a series of L motifs. An exemplary HIV-1 p6 is SEQ ID NO: 85.

A “profile” is used herein to indicate an aggregate of information regarding a preparation of cell or membrane surface proteins. A profile will comprise, at minimum, information regarding the presence or absence of such proteins. More typically, a profile will comprise information regarding the presence or absence of a plurality of such proteins. In addition, a profile may contain other information about each identified protein, such as relative or absolute amount of protein present, the degree of post-translational modification, membrane topology, three-dimensional structure, isoelectric point, molecular weight, etc. A “test profile” is a profile obtained from a subject of unknown diagnostic state. A “reference profile” is a profile obtained from subject known to be infected or uninfected.

The terms peptides, proteins and polypeptides are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “receptor” or “protein having a receptor function” is a protein that interacts with an extracellular ligand or a ligand that is within the cell but in a space that is topologically equivalent to the extracellular space (eg. inside the Golgi, inside the endoplasmic reticulum, inside the nuclear membrane, inside a lysosome or transport vesicle, etc.). Exemplary receptors are identified herein by annotation as such in various public databases. Receptors often have membrane domains.

A “recombinant nucleic acid” is any nucleic acid that has been placed adjacent to another nucleic acid by recombinant DNA techniques. A “recombined nucleic acid” also includes any nucleic acid that has been placed next to a second nucleic acid by a laboratory genetic technique such as, for example, tranformation and integration, transposon hopping or viral insertion. In general, a recombined nucleic acid is not naturally located adjacent to the second nucleic acid.

The term “recombinant protein” refers to a protein of the present invention which is produced by recombinant DNA techniques, wherein generally DNA encoding the expressed protein is inserted into a suitable expression vector which is in turn used to transform a host cell to produce the heterologous protein. Moreover, the phrase “derived from”, with respect to a recombinant gene encoding the recombinant protein is meant to include within the meaning of “recombinant protein” those proteins having an amino acid sequence of a native protein, or an amino acid sequence similar thereto which is generated by mutations including substitutions and deletions of a naturally occurring protein.

A “RING domain” or “Ring Finger” is a zinc-binding domain with a defined octet of cysteine and histidine residues. Certain RING domains comprise the consensus sequences as set forth below (amino acid nomenclature is as set forth in Table 1): Cys Xaa Xaa Cys Xaa₁₀₋₂₀ Cys Xaa His Xaa₂₋₅ Cys Xaa Xaa Cys Xaa₁₃₋₅₀ Cys Xaa Xaa Cys or Cys Xaa Xaa Cys Xaa₁₀₋₂₀ Cys Xaa His Xaa₂₋₅ His Xaa Xaa Cys Xaa₁₃₋₅₀ Cys Xaa Xaa Cys. Preferred RING domains of the invention bind to various protein partners to form a complex that has ubiquitin ligase activity. RING domains preferably interact with at least one of the following protein types: F box proteins, E2 ubiquitin conjugating enzymes and cullins.

A “scaffolding protein” is a protein that brings together two or more different proteins that interact to accomplish one or more particular functions. A scaffolding protein may, in addition to acting as a scaffold, carry out biochemical functions on its own or as part of a complex.

“Small molecule” as used herein, is meant to refer to a composition, which has a molecular weight of less than about 5 kD and most preferably less than about 2.5 kD. Small molecules can be nucleic acids, peptides, polypeptides, peptidomimetics, carbohydrates, lipids or other organic (carbon containing or inorganic molecules. Many pharmaceutical companies have extensive libraries of chemical and/or biological mixtures comprising arrays of small molecules, often fungal, bacterial, or algal extracts, which can be screened with any of the assays of the invention.

An “SH2” or “Src Homology 2” domain is a protein domain of generally about 100 amino acid residues. SH2 domains function as regulatory modules of intracellular signalling cascades by interacting with high affinity to phosphotyrosine-containing target peptides in a sequence-specific and phosphorylation-dependent manner.

An “SH3” or “Src Homology 3” domain is a protein domain of generally about 60 amino acid residues first identified as a conserved sequence in the non-catalytic part of several cytoplasmic protein tyrosine kinases (e.g. Src, Abl, Lck). SH3 domains mediate assembly of specific protein complexes via binding to proline-rich peptides.

As used herein, the term “specifically hybridizes” refers to the ability of a nucleic acid probe/primer of the invention to hybridize to at least 12, 15, 20, 25, 30, 35, 40, 45, 50 or 100 consecutive nucleotides of a target gene sequence, or a sequence complementary thereto, or naturally occurring mutants thereof, such that it has less than 15%, preferably less than 10%, and more preferably less than 5% background hybridization to a cellular nucleic acid (e.g., mRNA or genomic DNA) other than the target gene. A variety of hybridization conditions may be used to detect specific hybridization, and the stringency is determined primarily by the wash stage of the hybridization assay. Generally high temperatures and low salt concentrations give high stringency, while low temperatures and high salt concentrations give low stringency. Low stringency hybridization is achieved by washing in, for example, about 2.0×SSC at 50° C., and high stringency is acheived with about 0.2×SSC at 50° C. Further descriptions of stringency are provided below.

“STAM” proteins include a family of proteins involved in receptor mediated exo- and endocytosis as well as cellular signalling, generally. STAM proteins generally comprise an N-terminal VHS homology domain, a ubiquitin-interacting motif and an SH3 domain and optionally an immunoreceptor tyrosine-based activation motif. STAM 1 and STAM 2A are involved in cytokine-mediated signalling for DNA synthesis and c-myc induction. EAST and STAM 2A/Hbp play a role in receptor-mediated endo- and exocytosis and probably also in the regulation of actin cytoskeleton. (Lohi et al. FEBS Lett 2001 Nov. 23;508(3):287-90).

As applied to polypeptides, “substantial sequence identity” means that two peptide sequences, when optimally aligned, such as by the programs GAP or BESTFIT using default gap which share at least 90 percent sequence identity, preferably at least 95 percent sequence identity, more preferably at least 99 percent sequence identity or more. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

“Transcriptional regulatory sequence” is a generic term used throughout the specification to refer to DNA sequences, such as initiation signals, enhancers, and promoters, which induce or control transcription of protein coding sequences with which they are operably linked. In preferred embodiments, transcription of a recombinant protein gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of the protein.

A “UIM” domain is a ubiquitin binding motif.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “virion” is a complete viral particle; nucleic acid and capsid (and a lipid envelope in some viruses.

A “VHS” domain is a “Vps27p, Hrs and STAM” domain, named for the proteins in which it has been identified, and includes a DXXLL sequence motif. VHS domains have also been identified in the GGA (Golgi-localized, gamma-ear-containing, ADP-ribosylation-factor-binding) proteins. In certain embodiments, VHS domains of the invention recognize one or more acidic-cluster-dileucine signals found in the cytoplasmic tails of sorting receptors, such as mannose-6-phosphate receptors. (Misra et al. (2002) Nature 2002 Feb. 21;415(6874):933-7)

TABLE 1
Abbreviations for classes of amino acids*

| Symbol | Category | Amino Acids Represented |
|---|---|---|
| X1 | Alcohol | Ser, Thr |
| X2 | Aliphatic | Ile, Leu, Val |
| Xaa | Any | Ala, Cys, Asp, Glu, Phe, Gly, |
| | | His, Ile, Lys, Leu, Met, Asn, |
| | | Pro, Gln, Arg, Ser, Thr, Val, |
| | | Trp, Tyr |
| X4 | Aromatic | Phe, His, Trp, Tyr |
| X5 | Charged | Asp, Glu, His, Lys, Arg |
| X6 | Hydrophobic | Ala, Cys, Phe, Gly, His, Ile, |
| | | Lys, Leu, Met, Thr, Val, Trp, |
| | | Tyr |
| X7 | Negative | Asp, Glu |
| X8 | Polar | Cys, Asp, Glu, His, Lys, Asn, |
| | | Gln, Arg, Ser, Thr |
| X9 | Positive | His, Lys, Arg |
| X10 | Small | Ala, Cys, Asp, Gly, Asn, Pro, |
| | | Ser, Thr, Val |
| X11 | Tiny | Ala, Gly, Ser |
| X12 | Turnlike | Ala, Cys, Asp, Glu, Gly, His, |
| | | Lys, Asn, Gln, Arg, Ser, Thr |
| X13 | Asparagine-Aspartate | Asn, Asp |

*Abbreviations as adopted from http://smart.embl-heidelberg.de/SMART_DATA/alignments/consensus/grouping.html.

2. Overview

In certain aspects, the invention relates to the observation that AVMSP polypeptides are involved in viral maturation process such as assembly, budding and/or release. Any one AVMSP may be involved at one or more stages of viral maturation and may form one or more complexes with viral and/or host proteins.

In certain aspects, AVMSP sequences disclosed herein, and related methods and compositions may be used to manipulate a variety of significant biological processes. In certain embodiments, AVMSP nucleic acids and polypeptides of the invention are involved in a viral lifecycle. In one embodiment, an SH3 domain of a RING-SH3 protein binds to a viral Gag sequence represented by a consensus sequence P(T/S)AP, RXXP(T/S)AP or PXXDY, such as, for example, the PTAP or PFRDY sequences of HIV Gag (positioned, for example, at 455458 and 292-296, respectively).

In a further embodiment, AVMSP polypeptides may be involved in the formation of endocytosis-like or vesicle trafficking-like complexes that are involved in a stage of viral maturation. Such complexes may include complexes similar to those formed in connection with clathrin-mediated vesicle trafficking or those formed in connection with coatomer-coated vesicle trafficking.

Viral assembly, budding and release is expected to require a range of different protein complexes that incorporate host proteins involved in different aspects of vesicle trafficking, including vesicle formation proteins such as ARFs, COPs, RABs and clathrins, cytoskeletal proteins such as actins and myosins, cytoskeletal regulators such as Rac and Rho, heat shock proteins, STAM proteins and viral proteins, particularly proteins having a Late domain, such as Gag. Such complexes are expected to incorporate one or more AVMSPs, and particularly AVMSPs such as RING-SH3 proteins. At each stage of vesicle transport, the exact components of the relevant complexes may shift, but in general, it is understood that disrupting the ability of an AVMSP to participate in complex formation or dissolution will be effective in disrupting viral production. In certain embodiments, it is understood that interfering with AVMSP activity will not decrease the rate of viral production but will result in the production of defective viral particles having decreased ability to infect another host cell.

It is generally understood that AVMSPs, especially AVMSPs having similar functional domains, may exhibit substantial functional overlap in both native host functions and in the viral lifecycle. Accordingly, the invention provides methods for inhibiting a virus by interfering with the activity of more than one AVMSP. A preferred antiviral agent is able, as a single compound or mixture of compounds to interfere with more than one AVMSP.

3. Exemplary Nucleic Acids and Expression Vectors

In certain aspects the invention provides nucleic acids encoding Atemate Viral Maturation Scaffolding Proteins (AVMSPs)., such as for example RING-SH3 proteins, RING-SH2 proteins, RING-membrane proteins and RING-receptor proteins. There are four basic classes of AVMSPs: the RING-SH3 class, comprising at least one SH3 domain and at least one RING domain, the RING-SH2 class, comprising at least one SH2 domain and at least one RING domain, the RING-membrane class, comprising at least one RING domain and at least one transmembrane domain, and the RING-receptor class, comprising at least one RING domain and having receptor functionality. These four classes are not mutually exclusive, as, for example, a polypeptide may comprise a RING domain, and SH3 domain and an SH2 domain. In preferred embodiments, proteins of any of the four classes comprise at least one C2 domain.

Nucleic acids of the invention is further understood to include nucleic acids that encode variants of AVMSPs. Variant nucleotide sequences include sequences that differ by one or more nucleotide substitutions, additions or deletions, such as allelic variants; and will, therefore, include coding sequences that differ from the nucleotide sequence of the coding sequence designated in Tables 2-4 e.g., due to the degeneracy of the genetic code. Variants will also include nucleotide sequences that hybridize under stringent conditions (i.e., equivalent to about 20-27° C. below the melting temperature (Tₘ) of the DNA duplex formed in about 1M salt) to the nucleotide sequence of a coding sequence designated in Tables 2 and 3. Alternatively put, variants will also include nucleotide sequences that hybridize under moderately stringent conditions, for example at about 2.0×SSC and about 40° C. to the nucleotide sequence of a coding sequence designated in Tables 2-4. In another embodiment, equivalent nucleic acid sequences include sequences that will hybridize under highly stringent conditions to a nucleotide sequence of a coding sequence designated in Tables 2-4.

One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

In one embodiment, variants will further include nucleic acid sequences derived from and evolutionarily related to a nucleotide sequence of a coding sequence designated in Tables 2-4.

TABLE 2
Exemplary AVMSPs

| | Accession Nos. | Nucleotide | Protein | Selected Domains or |
|---|---|---|---|---|
| Name | Nucleotide; Protein | SEQ I.D. No. | SEQ I.D. No. | Features of Interest |
| Human PRT3 | | 40 | 1 | RING, SH3 |
| Coding sequence | | | | |
| Human PRT3 | | 41 | 1 | |
| cDNA | | | | |
| 5′ cDNA fragment of | | 42 | 2 | |
| human PRT3 | | | | |
| 3′mRNA fragment of | | 43 | 39 | |
| human PRT3 | | | | |
| SIMILAR TO MUS | EMBL; AK021429; | 44 | 3 | RING, SH3 |
| MUSCULUS | BAB13822.1 | | | |
| PLENTY OF SH3S | | | | |
| (POSH) | | | | |
| SH3-BINDING | EMBL; U26710; | 45 | 4 | RING, SH2 |
| PROTEIN CBL-B | AAB09291.1 | | | |
| SH3-BINDING | EMBL; AB028645; | 46 | 5 | RING, SH2 |
| PROTEIN CBL-C | BAA86298.1 | | | |
| MULTIPLE | EMBL; AF064801; | 47 | 6 | RING, Membrane |
| MEMBRANE | AAC39930.1 | | | |
| SPANNING | | | | |
| RECEPTOR TRC8 | | | | |
| AUTOCRINE | EMBL; AF124145; | 48 | 7 | RING, Membrane |
| MOTILITY FACTOR | AAD56722.1 | | | |
| RECEPTOR | | | | |
| MEMBRANE- | EMBL; AF255303; | 49 | 8 | RING, Membrane |
| ASSOCIATED | AAG00432.1 | | | |
| NUCLEIC ACID | | | | |
| BINDING PROTEIN | | | | |
| (FRAGMENT) | | | | |
| NADH- | EMBL; X81900; | 50 | 9 | RING, Membrane |
| UBIQUINONE | CAA57489.1 | | | |
| OXIDOREDUCTASE | | | | |
| MWFE SUBUNIT | | | | |
| PEROXISOME | EMBL; M86852; | 51 | 10 | RING, Membrane |
| ASSEMBLY | AAC12785.1 | | | |
| FACTOR-1 (PAF-1) | | | | |
| PEROXISOME | EMBL; AF060502; | 52 | 11 | RING, Membrane |
| ASSEMBLY | AAC18133.1 | | | |
| PROTEIN 10 | | | | |
| PEROXISOME | EMBL; U91521; | 53 | 12 | RING, Membrane |
| ASSEMBLY | AAC68812.1 | | | |
| PROTEIN 12 | | | | |
| ACETYLCHOLINE | EMBL; Z33905; | 54 | 13 | RING, Membrane |
| RECEPTOR- | CAA83954.1 | | | |
| ASSOCIATED 43 | | | | |
| KDA PROTEIN | | | | |
| PEROXISOMAL | EMBL; BC000661; | 55 | 14 | RING, Membrane |
| MEMBRANE | AAH00661.1 | | | |
| PROTEIN 3 | | | | |
| HsRmal | EMBL; AB056869; | 56 | 15 | RING, Membrane |
| | BAB39359.1 | | | |
| HTRIP | EMBL; U77845; | 57 | 16 | RING, Receptor |
| | AAB52993.1 | | | |
| CYSTEIN RICH | EMBL; X80200; | 58 | 17 | RING, Receptor |
| DOMAIN | CAA56491.1 | | | |
| ASSOCIATED TO | | | | |
| RING AND TRAF | | | | |
| PROTEIN | | | | |
| PML-RAR | EMBL; M73779; | 59 | 18 | RING, Receptor |
| | AAA60126.1 | | | |
| TRAF5 | EMBL; AB000509; | 60 | 19 | RING, Receptor |
| | BAA25262.1 | | | |
| TUMOR NECROSIS | EMBL; AF082185; | 61 | 20 | RING, Receptor |
| FACTOR | AAC32376.1 | | | |
| RECEPTOR- | | | | |
| ASSOCIATED | | | | |
| FACTOR 4A | | | | |
| HFB30 | EMBL; AB022663; | 62 | 21 | RING, Receptor |
| | BAA78677.1 | | | |
| BIR2 | EMBL; L49432; | 63 | 22 | RING, Receptor |
| | AAC41943.1 | | | |
| BIR3 | EMBL; L49431; | 64 | 23 | RING, Receptor |
| | AAC41942.1 | | | |
| CBL | EMBL; X57110; | 65 | 24 | RING, Receptor |
| | CAA40393.1 | | | |
| PML | EMBL; M79462; | 66 | 25 | RING, Receptor |
| | AAA60388.1 | | | |
| RAG1 | EMBL; M29474; | 67 | 26 | RING, Receptor |
| | AAA60248.1 | | | |
| RAPSYN | EMBL; Z33905; | 68 | 27 | RING, Receptor |
| | CAA83954.1 | | | |
| TIFI-ALPHA | EMBL; AF009353; | 69 | 28 | RING, Receptor |
| | AAB63585.1 | | | |
| TIFI-GAMMA | EMBL; AF119043; | 70 | 29 | RING, Receptor |
| | AAD17259.1 | | | |
| TRAF2 | EMBL; U12597; | 71 | 30 | RING, Receptor |
| | AAA87706.1 | | | |
| TRAF3 | EMBL; U21092; | 72 | 31 | RING, Receptor |
| | AAC50112.1 | | | |
| HIP116 | EMBL; L34673; | 73 | 32 | RING |
| | AAA67436.1 | | | |
| STAF50 | EMBL; X82200; | 74 | 33 | RING |
| | CAA57684.1 | | | |
| HLTF-1 | EMBL; Z46606; | 75 | 34 | RING |
| | CAA86571.1 | | | |
| BIR4 | EMBL; U45880; | 76 | 35 | RING |
| | AAC50373.1 | | | |
| ADENOVIRUS 5 | EMBL; X86098; | 77 | 36 | RING |
| E1A-BINDING | CAA60052.1 | | | |
| PROTEIN | | | | |
| HT2A | EMBL; U18543; | 78 | 37 | RING |
| | AAA86474.1 | | | |
| TRAF3 | EMBL; U21092; | 79 | 38 | RING |
| | AAC50112.1 | | | |

TABLE 3
Exemplary POSH nucleic acids

| | | Accession |
|---|---|---|
| Sequence Name | Organism | Number |
| cDNA FLJ11367 fis, clone | Homo sapiens | AK021429 |
| HEMBA1000303 | | |
| Plenty of SH3 domains | Mus musculus | NM_021506 |
| (POSH) mRNA | | |
| Plenty of SH3s (POSH) | Mus musculus | AF030131 |
| mRNA | | |
| Plenty of SH3s (POSH) | Drosophila melanogaster | NM_079052 |
| mRNA | | |
| Plenty of SH3s (POSH) | Drosophila melanogaster | AF220364 |
| mRNA | | |

TABLE 4
Exemplary POSH polypeptides

| | | Accession |
|---|---|---|
| Sequence Name | Organism | Number |
| SH3 domains- | Mus musculus | T09071 |
| containing protein POSH | | |
| plenty of SH3 domains | Mus musculus | NP_067481 |
| Plenty of SH3s; POSH | Mus musculus | AAC40070 |
| Plenty of SH3s | Drosophila melanogaster | AAF37265 |
| LD45365p | Drosophila melanogaster | AAK93408 |
| POSH gene product | Drosophila melanogaster | AAF57833 |
| Plenty of SH3s | Drosophila melanogaster | NP_523776 |

Isolated nucleic acids which differ from the nucleotide sequences encoding a protein designated in Tables 2-4 due to degeneracy in the genetic code are also within the scope of the invention. For example, a number of amino acids are designated by more than one triplet. Codons that specify the same amino acid, or synonyms (for example, CAU and CAC are synonyms for histidine) may result in, “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this invention.

Another aspect of the invention relates to the use of the isolated nucleic acid in “antisense” therapy. As used herein, antisense therapy refers to administration or in situ generation of oligonucleotide probes or their derivatives which specifically hybridize (e.g. binds) under cellular conditions with the cellular mRNA and/or genomic DNA encoding one of the subject AVMSPs so as to inhibit expression of that protein, e.g. by inhibiting transcription and/or translation. The binding may be by conventional base pair complementarity, or, for example, in the case of binding to DNA duplexes, through specific interactions in the major groove of the double helix. In general, antisense therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes an AVMSP. Alternatively, the antisense construct is an oligonucleotide probe which is generated ex vivo and which, when introduced into the cell causes inhibition of expression by hybridizing with the mRNA and/or genomic sequences encoding an AVMSP. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general.

In addition to use in therapy, the oligomers of the invention may be used as diagnostic reagents to detect the presence or absence of the target DNA or RNA sequences to, which they specifically bind, such as for determining the level of expression of a gene of the invention or for determining whether a gene of the invention contains a genetic lesion.

In another aspect of the invention, the subject nucleic acid is provided in an expression vector comprising a nucleotide sequence encoding a subject AVMSP polypeptide and operably linked to at least one regulatory sequence. Operably linked is intended to mean that the nucleotide sequence is linked to a regulatory sequence in a manner which allows expression of the nucleotide sequence. Regulatory sequences are art-recognized and are selected to direct expression of the polypeptide having an activity of an AVMSP. Accordingly, the term regulatory sequence includes promoters, enhancers and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an AVMSP. Such useful expression control sequences, include, for example, the early and late promoters of SV40, tet promoter, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

As will be apparent, the subject gene constructs can be used to cause expression of the subject AVMSP polypeptides in cells propagated in culture, e.g. to produce proteins or polypeptides, including fusion proteins or polypeptides, for purification.

This invention also pertains to a host cell transfected with a recombinant gene including a coding sequence for one or more of the subject AVMSP. The host cell may be any prokaryotic or eukaryotic cell. For example, a polypeptide of the present invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing the subject AVMSP polypeptides. For example, a host cell transfected with an expression vector encoding an AVMSP polypeptide can be cultured under appropriate conditions to allow expression of the polypeptide to occur. The polypeptide may be secreted and isolated from a mixture of cells and medium containing the polypeptide. Alternatively, the polypeptide may be retained cytoplasmically and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a preferred embodiment, the AVMSP is a fusion protein containing a domain which facilitates its purification, such as an AVMSP-GST fusion protein, AVMSP-cellulose binding domain fusion protein, etc.

A nucleotide sequence encoding an AVMSP can be used to produce a recombinant form of the protein via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial) cells, are standard procedures.

A recombinant AVMSP can be produced by ligating the cloned gene, or a portion thereof, into a vector suitable for expression in either prokaryotic cells, eukaryotic cells, or both. Expression vehicles for production of a recombinant AVMSP include plasmids and other vectors. For instance, suitable vectors for the expression of an AVMSP include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al., (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant AVMSP by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

It is well known in the art that a methionine at the N-terminal position can be enzymatically cleaved by the use of the enzyme methionine aminopeptidase (MAP). MAP has been cloned from E. coli (Ben-Bassat et al., (1987) J. Bacteriol. 169:751-757) and Salmonella typhimurium and its in vitro activity has been demonstrated on recombinant proteins (Miller et al., (1987) PNAS USA 84:2718-1722). Therefore, removal of an N-terminal methionine, if desired, can be achieved either in vivo by expressing such recombinant polypeptides in a host which produces MAP (e.g., E. coli or CM89 or S. cerevisiae), or in vitro by use of purified MAP (e.g., procedure of Miller et al.).

Alternatively, the coding sequences for the polypeptide can be incorporated as a part of a fusion gene including a nucleotide sequence encoding a different polypeptide. This type of expression system can be useful under conditions where it is desirable, e.g., to produce an immunogenic fragment of an AVMSP. For example, the VP6 capsid protein of rotavirus can be used as an immunologic carrier protein for portions of polypeptide, either in the monomeric form or in the form of a viral particle. The nucleic acid sequences corresponding to the portion of the AVMSP to which antibodies are to be raised can be incorporated into a fusion gene construct which includes coding sequences for a late vaccinia virus structural protein to produce a set of recombinant viruses expressing fusion proteins comprising a portion of the protein as part of the virion. The Hepatitis B surface antigen can also be utilized in this role as well. Similarly, chimeric constructs coding for fusion proteins containing a portion of an AVMSP and the poliovirus capsid protein can be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

The Multiple Antigen Peptide system for peptide-based immunization can be utilized, wherein a desired portion of an AVMSP is obtained directly from organo-chemical synthesis of the peptide onto an oligomeric branching lysine core (see, for example, Posnett et al., (1988) JBC 263:1719 and Nardelli et al., (1992) J. Immunol. 148:914). Antigenic determinants of an AVMSP can also be expressed and presented by bacterial cells.

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified AVMSP (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

4. Exemplary Polypeptides

The present invention also makes available isolated and/or purified forms of the subject AVMSPs, which are isolated from, or otherwise substantially free of, other intracellular proteins which might normally be associated with the protein or a particular complex including the protein. In certain embodiments, polypeptides of the invention have an amino acid sequence that is at least 60% identical to an amino acid sequence as set forth in SEQ ID Nos. 1-39. In other embodiments, the polypeptide ha an amino acid sequence at least 65%, 70%, 75%, 80%, 85%, 90%, 95%, 97%, 98%, 99% or 100% identical to an amino acid sequence as set forth in SEQ ID Nos. 1-39.

In another aspect, the invention provides polypeptides that are agonists or antagonists of AVMSPs. Variants and fragments of an AVMSP may have a hyperactive or constitutive activity, or, alternatively, act to prevent AVMSPs from performing one or more functions. For example, a truncated form lacking one or more domain may have a dominant negative effect.

Another aspect of the invention relates to polypeptides derived from a full-length AVMSP. Isolated peptidyl portions of the subject proteins can be obtained by screening polypeptides recombinantly produced from the corresponding fragment of the nucleic acid encoding such polypeptides. In addition, fragments can be chemically synthesized using techniques known in the art such as conventional Merrifield solid phase f-Moc or t-Boc chemistry. For example, any one of the subject proteins can be arbitrarily divided into fragments of desired length with no overlap of the fragments, or preferably divided into overlapping fragments of a desired length. The fragments can be produced (recombinantly or by chemical synthesis) and tested to identity those peptidyl fragments which can function as either agonists or antagonists of the formation of a specific protein complex, or more generally of an AVMSP, such as by microinjection assays.

It is also possible to modify the structure of the subject AVMSPs for such purposes as enhancing therapeutic or prophylactic efficacy, or stability (e.g., ex vivo shelf life and resistance to proteolytic degradation in vivo). Such modified polypeptides, when designed to retain at least one activity of the naturally-occurring form of the protein, are considered functional equivalents of the AVMSPs described in more detail herein. Such modified polypeptides can be produced, for instance, by amino acid substitution, deletion, or addition.

For instance, it is reasonable to expect, for example, that an isolated replacement of a leucine with an isoleucine or valine, an aspartate with a glutamate, a threonine with a serine, or a similar replacement of an amino acid with a structurally related amino acid (i.e. conservative mutations) will not have a major effect on the biological activity of the resulting molecule. Conservative replacements are those that take place within a family of amino-acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2nd ed., Ed. by L. Stryer, W.H. Freeman and Co., 1981). Whether a change in the amino acid sequence of a polypeptide results in a functional homolog can be readily determined by assessing the ability of the variant polypeptide to produce a response in cells in a fashion similar to the wild-type protein. For instance, such variant forms of an AVMSP can be assessed, e.g., for their ability to bind to another polypeptide, e.g., another AVMSP or another protein involved in viral maturation. Polypeptides in which more than one replacement has taken place can readily be tested in the same manner.

This invention further contemplates a method of generating sets of combinatorial mutants of the subject AVMSPs, as well as truncation mutants, and is especially useful for identifying potential variant sequences (e.g. homologs) that are functional in binding to an AVMSP. The purpose of screening such combinatorial libraries is to generate, for example, AVMSP homologs which can act as either agonists or antagonist, or alternatively, which possess novel activities all together. Combinatorially-derived homologs can be generated which have a selective potency relative to a naturally occurring AVMSP. Such proteins, when expressed from recombinant DNA constructs, can be used in gene therapy protocols.

Likewise, mutagenesis can give rise to homologs which have intracellular half-lives dramatically different than the corresponding wild-type protein. For example, the altered protein can be rendered either more stable or less stable to proteolytic degradation or other cellular process which result in destruction of, or otherwise inactivation of the AVMSP of interest. Such homologs, and the genes which encode them, can be utilized to alter AVMSP expression by modulating the half-life of the protein. For instance, a short half-life can give rise to more transient biological effects and, when part of an inducible expression system, can allow tighter control of recombinant AVMSP levels within the cell. As above, such proteins, and particularly their recombinant nucleic acid constructs, can be used in gene therapy protocols.

In similar fashion, AVMSP homologs can be generated by the present combinatorial approach to act as antagonists, in that they are able to interfere with the ability of the corresponding wild-type protein to function.

In a representative embodiment of this method, the amino acid sequences for a population of AVMSP homologs are aligned, preferably to promote the highest homology possible. Such a population of variants can include, for example, homologs from one or more species, or homologs from the same species but which differ due to mutation. Amino acids which appear at each position of the aligned sequences are selected to create a degenerate set of combinatorial sequences. In a preferred embodiment, the combinatorial library is produced by way of a degenerate library of genes encoding a library of polypeptides which each include at least a portion of potential AVMSP sequences. For instance, a mixture of synthetic oligonucleotides can be enzymatically ligated into gene sequences such that the degenerate set of potential AVMSP nucleotide sequences are expressible as individual polypeptides, or alternatively, as a set of larger fusion proteins (e.g. for phage display).

There are many ways by which the library of potential homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential AVMSP sequences. The synthesis of degenerate oligonucleotides is well known in the art (see for example, Narang, SA (1983) Tetrahedron 39:3; Itakura et al., (1981) Recombinant DNA, Proc. 3rd Cleveland Sympos. Macromolecules, ed. AG Walton, Amsterdam: Elsevier pp 273-289; Itakura et al., (1984) Annu. Rev. Biochem. 53:323; Itakura et al., (1984) Science 198:1056; Ike et al., (1983) Nucleic Acid Res. 11:477). Such techniques have been employed in the directed evolution of other proteins (see, for example, Scott et al., (1990) Science 249:386-390; Roberts et al., (1992) PNAS USA 89:2429-2433; Devlin et al., (1990) Science 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, AVMSP homologs (both agonist and antagonist forms) can be generated and isolated from a library by screening using, for example, alanine scanning mutagenesis and the like (Ruf et al., (1994) Biochemistry 33:1565-1572; Wang et al., (1994) J. Biol. Chem. 269:3095-3099; Balint et al., (1993) Gene 137:109-118; Grodberg et al., (1993) Eur. J. Biochem. 218:597-601; Nagashima et al., (1993) J. Biol. Chem. 268:2888-2892; Lowman et al., (1991) Biochemistry 30:10832-10838; and Cunningham et al., (1989) Science 244:1081-1085), by linker scanning mutagenesis (Gustin et al., (1993) Virology 193:653-660; Brown et al., (1992) Mol. Cell Biol. 12:2644-2652; McKnight et al., (1982) Science 232:316); by saturation mutagenesis (Meyers et al., (1986) Science 232:613); by PCR mutagenesis (Leung et al., (1989) Method Cell Mol Biol 1:11-19); or by random mutagenesis, including chemical mutagenesis, etc. (Miller et al., (1992) A Short Course in Bacterial Genetics, CSHL Press, Cold Spring Harbor, N.Y.; and Greener et al., (1994) Strategies in Mol Biol 7:32-34). Linker scanning mutagenesis, particularly in a combinatorial setting, is an attractive method for identifying truncated (bioactive) forms of AVMSPs.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of AVMSP homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate sequences created by combinatorial mutagenesis techniques.

In an illustrative embodiment of a screening assay, candidate combinatorial gene products of one of the subject proteins are displayed on the surface of a cell or virus, and the ability of particular cells or viral particles to bind an AVMSP, eg. a protein designated in Tables 2 and 3, is detected in a “panning assay”. For instance, a library of AVMSP-IP variants can be cloned into the gene for a surface membrane protein of a bacterial cell (Ladner et al., WO 88/06630; Fuchs et al., (1991) Bio/Technology 9:1370-1371; and Gowardi et al., (1992) TIBS 18:136-140), and the resulting fusion protein detected by panning, e.g. using a fluorescently labeled molecule which binds the AVMSP-IP, such as FITC-labelled AVMSP, to score for potentially functional homologs. Cells can be visually inspected and separated under a fluorescence microscope, or, where the morphology of the cell permits, separated by a fluorescence-activated cell sorter.

In similar fashion, the gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and f1 are most often used in phage display libraries, as either of the phage gIII or gVIII coat proteins can be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461).

The invention also provides for reduction of the subject AVMSPs to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of an AVMSP which participate in protein-protein interactions involved in, for example, binding of proteins involved in viral maturation to each other. To illustrate, the critical residues of an AVMSP which are involved in molecular recognition of a substrate protein can be determined and used to generate AVMSP-derived peptidomimetics which bind to the substrate protein, and by inhibiting AVMSP binding, act to inhibit its biological activity. By employing, for example, scanning mutagenesis to map the amino acid residues of an AVMSP which are involved in binding to another polypeptide, peptidomimetic compounds can be generated which mimic those residues involved in binding. For instance, non-hydrolyzable peptide analogs of such resides can be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988); substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., (1985) Tetrahedron Lett 26:647; and Sato et al., (1986) J Chem Soc. Perlin Trans 1:1231), and b-aminoalcohols (Gordon et al., (1985) Biochem Biophys Res Commun 126:419; and Dann et al., (1986) Biochem Biophys Res Commun 134:71).

5. Antibodies and Uses Therefor

Another aspect of the invention pertains to an antibody specifically reactive with an AVMSP, e.g., a wild-type or mutated AVMSP. For example, by using immunogens derived from an AVMSP, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a mammalian AVMSP or an antigenic fragment which is capable of eliciting an antibody response, or a fusion protein as described above). Techniques for conferring immunogenicity on a protein or peptide include conjugation to carriers or other techniques well known in the art. An immunogenic portion of an AVMSP can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of an AVMSP of a mammal, e.g., antigenic determinants of a protein set forth in SEQ ID Nos: 1-39.

In one embodiment, antibodies are specific for a RING domain, an SH3 domain, an SH2 domain, and a C2 domain, and preferably the domain is part of an AVMSP, such as a domain of an AVMSP shown in one of SEQ ID NOs: 1-39. In another embodiment, the antibodies are immunoreactive with one or more proteins having an amino acid sequence that is at least 80% identical to an amino acid sequence as set forth in SEQ ID Nos. 1-39. In other embodiment, an antibody is immunoreactive with one or more proteins having an amino acid sequence that is 85%, 90%, 95%, 98%, 99% or identical to an amino acid sequence as set forth in SEQ ID Nos. 1-39.

In a further embodiment, an antibody of the invention disrupts the direct or indirect interaction between an AVMSP polypeptide and an AVMSP-AP.

Following immunization of an animal with an antigenic preparation of an AVMSP, anti-AVMSP antisera can be obtained and, if desired, polyclonal anti-AVMSP antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian AVMSP polypeptide of the present invention and monoclonal antibodies isolated from a culture comprising such hybridoma cells. In one embodiment anti-human AVMSP antibodies specifically react with the protein encoded by a nucleic acid having SEQ ID Nos 40-79.

The term antibody as used herein is intended to include fragments thereof which are also specifically reactive with one of the subject mammalian AVMSP polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an AVMSP protein conferred by at least one CDR region of the antibody. In preferred embodiments, the antibodies, the antibody further comprises a label attached thereto and able to be detected, (e.g., the label can be a radioisotope, fluorescent compound, enzyme or enzyme co-factor).

Anti-AVMSP antibodies can be used, e.g., to monitor AVMSP levels in an individual, particularly the presence of AVMSPs in the plasma membrane for determining whether or not said patient is infected with a virus such as an RNA virus, or allowing determination of the efficacy of a given treatment regimen for an individual afflicted with such a disorder. In addition, AVMSPs are understood to localize, occasionally, to the released viral particle. Viral particles may be collected and assayed for the presence of one or more AVMSPs. The level of AVMSP may be measured in a variety of sample types such as, for example, cells and/or in bodily fluid, such as in blood samples.

Another application of anti-AVMSP antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of an AVMSP, e.g., other orthologs of a particular protein or other paralogs from the same species, can then be detected with antibodies, as, for example, reacting nitrocellulose filters lifted from infected plates with the appropriate anti-AVMSP antibodies. Positive phage detected by this assay can then be isolated from the infected plate. Thus, the presence of AVMSP homologs can be detected and cloned from other animals, as can alternate isoforms (including splice variants) from humans.

6. Homology Searching of Nucleotide and Polypeptide Sequences

The nucleotide or amino acid sequences of the invention may be used as query sequences against databases such as GenBank, SwissProt, BLOCKS, and Pima II. These databases contain previously identified and annotated sequences that can be searched for regions of homology (similarity) using BLAST, which stands for Basic Local Alignment Search Tool (Altschul S F (1993) J Mol Evol 36:290-300; Altschul, S F et al (1990) J Mol Biol 215:403-10).

BLAST produces alignments of both nucleotide and amino acid sequences to determine sequence similarity. Because of the local nature of the alignments, BLAST is especially useful in determining exact matches or in identifying homologs which may be of prokaryotic (bacterial) or eukaryotic (animal, fungal or plant) origin. Other algorithms such as the one described in Smith, R. F. and T. F. Smith (1992; Protein Engineering 5:35-51), incorporated herein by reference, can be used when dealing with primary sequence patterns and secondary structure gap penalties. As disclosed in this application, sequences have lengths of at least 49 nucleotides and no more than 12% uncalled bases (where N is recorded rather than A, C, G, or T).

The BLAST approach, as detailed in Karlin and Altschul (1993; Proc Nat Acad Sci 90:5873-7) and incorporated herein by reference, searches matches between a query sequence and a database sequence, to evaluate the statistical significance of any matches found, and to report only those matches which satisfy the user-selected threshold of significance. Preferably the threshold is set at 10-25 for nucleotides and 3-15 for peptides.

7. Diagnostic Assays

A further aspect of the invention includes diagnostic assays for determining whether a cell is infected with a virus and for characterizing the nature, progression and/or infectivity of the infection.

In one embodiment, it is contemplated that AVMSPs certain associated proteins localize to different regions of the cell depending on the function being performed. In the course of normal activities, it is expected that AVMSPs will be free in the cytoplasm or associated with an intracellular organelle, such as the nucleus, the Golgi network, etc. During a viral infection, certain AVMSPs are recruited to the cell membrane to participate in viral maturation, including ubiquitination and membrane fusion. As a result, the detection of an AVMSP associated with the plasma membrane fraction is indicative of a viral infection. Additionally, the presence of AVMSPs at the plasma membrane would suggest that the infective virus is in the process of reproducing and is therefore actively engaged in infective or lytic activity (versus a lysogenic or otherwise dormant state).

Association of the proteins of the invention with the plasma membrane may be detected using a variety of techniques known in the art. For example, membrane preparations may be prepared by breaking open the cells (via sonication or detergent lysis) and then separating the membrane components from the cytosolic fraction via centrifugation. Segregation of proteins into the membrane fraction can be detected with antibodies specific for the protein of interest, for example by using Western blot analysis or ELISA techniques. Plasma membranes may be separated from intracellular membranes on the basis of density using density gradient centrifugation. Alternatively, plasma membranes may be obtained by chemically or enzymatically modifying the surface of the cell and affinity purifying the plasma membrane by selectively binding the modifications. An exemplary modification includes non-specific biotinylation of proteins at the cell surface. Plasma membranes may also be selected for by affinity purifying for abundant plasma membrane proteins.

Transmembrane AVMSP proteins containing an extracellular domain can be detected using FACS analysis. For FACS analysis, whole cells are incubated with a fluorescently labeled antibody (e.g., an FITC-labelled antibody) capable of recognizing the extracellular domain of the protein of interest. The level of fluorescent staining of the cells may then be determined by FACS analyses (see e.g., Weiss and Stobo, (1984) J. Exp. Med., 160:1284-1299). Such proteins are expected to reside on intracellular membranes in uninfected cells and the plasma membrane in infected cells. FACS analysis would fail to detect an extracellular domain unless the protein is present at the plasma membrane.

In a further embodiment, proteins associated with the membranes of cells and/or viral particles may be profiled. Profiling involves identifying the presence or absence of more than one protein in the membrane associated fraction of a sample. For example, the presence of AVMSPs are detected in the membrane associated fraction of cells obtained from a person suspected of a viral infection. Similar profiles may be developed from subjects infected by known viruses or subjects thought to be free of infection. Profiles may be compared to identify proteins that change in abundance, or qualitatively (eg. in terms of PL molecular weight, or other indicators of post-translational modification). Profiles may be compiled into a database for computer-assisted comparisons. Comparison of profiles may be used to identify an AVMSP that is altered in response to a certain viral infection. This AVMSP may then be used as a diagnostic for that type of viral infection. The AVMSP may also then be used as a target to identify therapeutic agents that will interfere with its function in the infection. Exemplary profiles of the invention will include information about the abundance of more than one AVMSP selected from those represented by SEQ ID Nos. 1-39. Other exemplary profiles will include information about the abundance of 5, 10, 20, 30, 40, 50, 60 or all of the proteins represented by SEQ ID Nos.1-39.

Localization of the proteins of the invention may also be determined using histochemical techniques. For example, cells may be fixed and stained with a fluorescently labeled antibody specific for the protein of interest. The stained cells may then be examined under the microscope to determine the subcellular localization of the antibody bound proteins.

In addition, as noted above, AVMSPs may localize to released or budding viral particles. The presence of these proteins in viral particles may be determined by a variety of methods. For example, viral particles may be enriched and analyzed by Western blot or ELISA. As another example, viral particles or cells having budding viroids ay be examined by electron microscopy. Immunogold labeling, for example, is useful for localizing AVMSPs by electron microscopy.

Samples to be used for diagnostic assays may include essentially any sample comprising cells and/or viral particles or a sample prepared from a cellular sample. Exemplary samples would include fluid samples (eg. blood, urine, saliva, mucus, broncheoalveolar lavage, cerebrospinal fluid etc.). Other fluids comprising cells and/or viral particles are well known to those of skill in the art. Other sample types include stool samples, tissue biopsies and any processed or purified form of the above.

8. Drug Screening Assays

The present invention also provides assays for identifying therapeutics which either interfere with or promote viral maturation, particularly by affecting AVMSP function. In one embodiment, the assay detects agents which inhibit interaction of one or more subject AVMSPs with an AVMSP-AP. In another embodiment, the assay detects agents which modulate the intrinsic biological activity of an AVMSP, AVMSP complex, such as an enzymatic activity, binding to other cellular components, cellular compartmentalization, and the like. Such modulators can be used, for example, in the treatment of viral infections and/or particularly viral infections by a virus that uses a Gag-dependent maturation system (eg. retrovirus, rhabdovirus, filovirus).

In one aspect, the invention provides methods and compositions for the identification of compositions that interfere with the function of AVMSPs. Given the critical role of AVMSPs in virion release, compositions that perturb the formation or stability of the protein-protein interactions between AVMSPs and the proteins that they interact with, such as AVMSP-APs, are candidate pharmaceuticals for the treatment of viral infections.

While not wishing to be bound to mechanism, it is postulated that AVMSPs promote the assembly of protein complexes that are critically important in release of virions. Complexes of the invention may include a combination of at least one of the following: a polypeptide comprising an AVMSP, a Gag protein, a Gag late domain, PI3K, actin, myosin, Hsp60, Hsp70, Hsp90, STAM1, STAM2A, STAM2B, VHS-UIM, a GTPase, an E2 enzyme, tsg101, a cullin, AP-1, AP-2, and a clathrin.

The type of complex formed by an AVMSP will depend upon the domains present in the protein. While not intended to be limiting, exemplary domains of potential interacting proteins are provided below. An SH3 domain is expected to interact with one or more small GTPases, such as members of the Arf, Rab, Rac and Rho families. In addition, the following Table provides a list of exemplary protein domains that are associated with the formation of AVMSP complexes:

TABLE 5

| | Domain Name | Interacting motif | Description |
|---|---|---|---|
| 1 | SH2 | Yxxφ | The Src homology 2 (SH2) domain is a protein domain |
| | | | of about 100 amino-acid residues first identified as a |
| | | | conserved sequence region between the oncoproteins Src |
| | | | and Fps. Similar sequences were later found in many |
| | | | other intracellular signal-transducing proteins. SH2 |
| | | | domains function as regulatory modules of intracellular |
| | | | signalling cascades by interacting with high affinity to |
| | | | phosphotyrosine-containing target peptides in a |
| | | | sequence-specific and strictly phosphorylation-dependent |
| | | | manner. |
| 2 | SH3 | PxRPxR (proline | The Src homology 3 (SH3) domain is a small protein |
| | | rich) | domain of about 60 amino-acid residues first identified as |
| | | | a conserved sequence in the non-catalytic part of several |
| | | | cytoplasmic protein tyrosine kinases (e.g. Src, Abl, Lck). |
| | | | Since then, it has been found in a great variety of other |
| | | | intracellular or membrane-associated proteins. The |
| | | | function of the SH3 domain is to mediate assembly of |
| | | | specific protein complexes via binding to proline-rich |
| | | | peptides. |
| 3 | Endocytosis | Yxxφ | Tyrosine not phosphorylated |
| | motifs: | (D/E)xxxLL | |
| | μ2 subunit of | (dileucine) | |
| | AP2 | FYRAL | |
| | βadaptin or | NPXY | |
| | through an | | |
| | adaptor to AP2 | | |
| | (NEF) | | |
| | AP2? | | |
| | Clatherin | | |
| 4 | Clatherin | Multiple DLL or | AP-2, AP180, AP1 |
| | assembly | SLL | |
| | domain | | |
| 5 | C2 | Ca2+, | Ca2+-binding motif present in phospholipases, protein |
| | | phospholipids, | kinases C, and synaptotamins (among others). Some do |
| | | inositol | not appear to contain Ca2+-binding sites. Particular C2s |
| | | polyphosphates. | appear to bind phospholipids, inositol polyphosphates, |
| | | | and intracellular proteins. Unusual occurrence in |
| | | | perforin. Synaptotagmin and PLC C2s are permuted in |
| | | | sequence with respect to N- and C-terminal beta strands. |
| 6 | WW | [AP]-P-P-[AP]-Y | Found in dystrophin. The domain, which spans about 35 |
| | | | residues, is repeated up to 4 times in some proteins. It |
| | | | has been shown to bind proteins with particular |
| | | | proline-motifs, and thus resembles somewhat SH3 |
| | | | domains. The name WW or WWP derives from the |
| | | | presence of Trp as well as that of a conserved Pro. It is |
| | | | frequently associated with other domains typical for |
| | | | proteins in signal transduction processes. |
| 7 | RCC1 | Ran GTPase | The regulator of chromosome condensation (RCC1) is a |
| | | | eukaryotic protein, which binds to chromatin and |
| | | | interacts with ran, a nuclear GTP-binding protein to |
| | | | promote the loss of bound GDP and the uptake of fresh |
| | | | GTP, thus acting as a guanine-nucleotide dissociation |
| | | | stimulator (GDS). |

A variety of assay formats will suffice and, in light of the present disclosure, those not expressly described herein will nevertheless be comprehended by one of ordinary skill in the art. Assay formats which approximate such conditions as formation of protein complexes, enzymatic activity, and even an AVMSP-mediated membrane reorganization activity, can be generated in many different forms, and include assays based on cell-free systems, e.g. purified proteins or cell lysates, as well as cell-based assays which utilize intact cells. Simple binding assays can also be used to detect agents which, by disrupting the binding of AVMSPs to interacting protein, or the binding of an AVMSP or complex to a substrate, can inhibit viral maturation. Agents to be tested for their ability to act as viral maturation inhibitors can be produced, for example, by bacteria, yeast or other organisms (e.g. natural products), produced chemically (e.g. small molecules, including peptidomiimetics), or produced recombinantly. In a preferred embodiment, the test agent is a small organic molecule, e.g., other than a peptide or oligonucleotide, having a molecular weight of less than about 2,000 daltons.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In preferred in vitro embodiments of the present assay, a reconstituted AVMSP complex comprises a reconstituted mixture of at least semi-purified proteins. By semi-purified, it is meant that the proteins utilized in the reconstituted mixture have been previously separated from other cellular or viral proteins. For instance, in contrast to cell lysates, the proteins involved in AVMSP complex formation, are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure AVMSP complex assembly and/or disassembly.

Assaying AVMSP complexes, in the presence and absence of a candidate inhibitor, can be accomplished in any vessel suitable for containing the reactants. Examples include microtitre plates, test tubes, and micro-centrifuge tubes.

In one embodiment of the present invention, drug screening assays can be generated which detect inhibitory agents on the basis of their ability to interfere with assembly or stability of the AVMSP complex. In an exemplary binding assay, the compound of interest is contacted with a mixture comprising an AVMSP polypeptide and is at least one interacting polypeptide. Detection and quantification of AVMSP complexes provides a means for determining the compound's efficacy at inhibiting (or potentiating) interaction between the two polypeptides. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

Complex formation between the AVMSP polypeptides or between an AVMSP and a substrate polypeptide may be detected by a variety of techniques, many of which are effectively described above. For instance, modulation in the formation of complexes can be quantitated using, for example, detectably labeled proteins (e.g. radiolabeled, fluorescently labeled, or enzymatically labeled), by immunoassay, or by chromatographic detection. Surface plasmon resonance systems, such as those available from BioCore, Inc., may also be used to detect protein-protein interaction Often, it will be desirable to immobilize one of the polypeptides to facilitate separation of complexes from uncomplexed forms of one of the proteins, as well as to accommodate automation of the assay. In an illustrative embodiment, a fusion protein can be provided which adds a domain that permits the protein to be bound to an insoluble matrix. For example, GST-AVMSP or -AMVSP fusion proteins can be adsorbed onto glutathione sepharose beads (Sigma Chemical, St. Louis, Mo.) or glutathione derivatized microtitre plates, which are then combined with a potential interacting protein, e.g. an ³⁵S-labeled polypeptide, and the test compound and incubated under conditions conducive to complex formation. Following incubation, the beads are washed to remove any unbound interacting protein, and the matrix bead-bound radiolabel determined directly (e.g. beads placed in scintillant), or in the supernatant after the complexes are dissociated, e.g. when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In yet another embodiment, the AVMSP and potential interacting polypeptide can be used to generate an interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the proteins to one and other.

In particular, the method makes use of chimeric genes which express hybrid proteins. To illustrate, a first hybrid gene comprises the coding sequence for a DNA-binding domain of a transcriptional activator can be fused in frame to the coding sequence for a “bait” protein, e.g., an AVMSP polypeptide of sufficient length to bind to a potential interacting protein. The second hybrid protein encodes a transcriptional activation domain fused in frame to a gene encoding a “fish” protein, e.g., a potential interacting protein of sufficient length to interact with the AVMSP polypeptide portion of the bait fusion protein. If the bait and fish proteins are able to interact, e.g., form an AVMSP complex, they bring into close proximity the two domains of the transcriptional activator. This proximity causes transcription of a reporter gene which is operably linked to a transcriptional regulatory site responsive to the transcriptional activator, and expression of the reporter gene can be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector. Interaction trap assays may also be performed in mammalian and bacterial cell types.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as an AVMSP polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, lcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300).

In preferred embodiments, any enzymatic activity associated with the bait or fish proteins is inactivated, e.g., dominant negative or other mutants of an AVMSP can be used.

Continuing with the illustrated example, the AVMSP-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of an AVMSP/interacting protein complex results in a detectable signal produced by the expression of the reporter gene. Accordingly, the level of formation of a complex in the presence of a test compound and in the absence of the test compound can be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations including an AVMSP and one or more interacting polypeptides.

In still further embodiments of the present assay, the AVMSP complex is generated in whole cells, taking advantage of cell culture techniques to support the subject assay. For example, as described below, the AVMSP complex can be constituted in a eukaryotic cell culture system, including mammalian and yeast cells. Often it will be desirable to express one or more viral proteins (eg. Gag or Env) in such a cell along with a subject AVMSP. It may also be desirable to infect the cell with a virus of interest. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the AVMSP can be endogenous to the cell selected to support the assay. Alternatively, some or all of the components can be derived from exogenous sources. For instance, fusion proteins can be introduced into the cell by recombinant techniques (such as through the use of an expression vector), as well as by microinjecting the fusion protein itself or mRNA encoding the fusion protein.

In any case, the cell is ultimately manipulated after incubation with a candidate drug and assayed for an AVMSP activity. AVMSP activities may include, without limitation, complex formation, ubiquitination and membrane fusion events (eg. release of viral buds or fusion of vesicles). AVMSP complex formation may be assessed by immunoprecipitation and analysis of co-immunoprecipiated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

To perform FRET-based assays, the AVMSP and the interacting protein of interest are both fluorescently labeled. Suitable fluorescent labels are, in view of this specification, well known in the art. Examples are provided below, but suitable fluorescent labels not specifically discussed are also available to those of skill in the art. Fluorescent labeling may be accomplished by expressing a polypeptide as a fusion protein with a fluorescent protein, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra Certain variants are altered such that they no longer appear green, and may appear blue, cyan, yellow or red (termed BFP, CFP, YFP and RFP, respectively). Fluorescent proteins may be stably attached to polypeptides through a variety of covalent and noncovalent linkages, including, for example, peptide bonds (eg. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol, 35:803-808; Levine et al. (1982) Comp. Biochem. Physiol., 72B:77-85; Tersikh et al. (2000) Science 290: 1585-88.

Other exemplary fluorescent moieties well known in the art include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri (Baldwin et al. (1990) Biochemistry 29:5509-15), peridinin-chlorophyll a binding protein from the dinoflagellate Symbiodinium sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

FRET-based assays may be used in cell-based assays and in cell-free assays. FRET-based assays are amenable to high-throughput screening methods including Fluorescence Activated Cell Sorting and fluorescent scanning of microtiter arrays.

10. Methods and Compositions for Treatment of Viral Disorders

In a further aspect, the invention provides methods and compositions for treatment of viral disorders, and particularly disorders caused by RNA viruses, including but not limited to retroviruses, rhabdoviruses and filoviruses. Preferred therapeutics of the invention function by disrupting the biological activity of an AVMSP or AVMSP complex in viral maturation.

Exemplary therapeutics of the invention include antisense therapies, RNAi or siRNA therapies, polypeptides, peptidomimetics, antibodies and small molecules.

Antisense therapies of the invention include methods of introducing antisense nucleic acids to disrupt the expression of AVMSPs or proteins that are necessary for AVMSP function. RNAi or siRNA therapies both refer to the administration of short double stranded RNA molecules that are complementary to a portion of an AVMSP transcript to achieve a decrease in the expression of that AVMSP and, thereby, a decrease in the function of that AVMSP. RNA molecules for siRNA may be prepared as a therapeutic composition by, for example, mixing them with a suitable carrier, such as a lipid or polycationic carrier that facilitates delivery of the nucleic acid across the membrane of the targeted cells.

Therapeutic polypeptides may be generated by designing polypeptides to mimic certain protein domains important in the formation of AVMSP complexes. For example, a polypeptide comprising an SH3 domain will compete for binding to an SH3 domain and will therefore act to disrupt binding of a Gag protein, for example, to the AVMSP complex. Likewise, a polypeptide that resembles an L domain may disrupt recruitment of Gag to the AVMSP complex. Such polypeptide mimetics may be targeted to any of a variety of domains, including for example, those domains listed in Table 5.

In view of the specification, methods for generating antibodies directed to epitopes of AVMSPs and AVMSP-interacting proteins are known in the art. Antibodies may be introduced into cells by a variety of methods. One exemplary method comprises generating a nucleic acid encoding a single chain antibody that is capable of disrupting an AVMSP complex. Such a nucleic acid may be conjugated to antibody that binds to receptors on the surface of target cells. It is contemplated that in certain embodiments, the antibody may target viral proteins that are present on the surface of infected cells, and in this way deliver the nucleic acid only to infected cells. Once bound to the target cell surface, the antibody is taken up by endocytosis, and the conjugated nucleic acid is transcribed and translated to produce a single chain antibody that interacts with and disrupts the targeted AVMSP complex. Nucleic acids expressing the desired single chain antibody may also be introduced into cells using a variety of more conventional techniques, such as viral transfection (eg. using an adenoviral system) or liposome-mediated transfection.

Small molecules of the invention may be identified for their ability to modulate the formation of AVMSP complexes, as described above.

In view of the teachings herein, one of skill in the art will understand that the methods and compositions of the invention are applicable to a wide range of RNA viruses including retroviruses. While not intended to be limiting, relevant retroviruses include: C-type retrovirus which causes lymphosarcoma in Northern Pike, the C-type retrovirus which infects mink, the caprine lentivirus which infects sheep, the Equine Infectious Anemia Virus EIAV, the C-type retrovirus which infects pigs, the Avian Leukosis Sarcoma Virus (ALSV), the Feline Leukemia Virus (FeLV), the Feline Aids Virus, the Bovine Leukemia Virus (BLV), the Simian Leukemia Virus (SLV), the Simian Immuno-deficiency Virus (SIV), the Human T-cell Leukemia Virus type-I (HTLV-I), the Human T-cell Leukemia Virus type-II (HTLV-I), Human Immunodeficiency virus type-2 (HIV-2) and Human Immunodeficiency virus type-1 (HIV-1). Other RNA viruses include picornaviruses such as enterovirus, poliovirus, coxsackievirus and hepatitis A virus, the caliciviruses, including Norwalk-like viruses, the rhabdoviruses, including rabies virus, the togaviruses including alphaviruses, Semliki Forest virus, denguevirus, yellow fever virus and rubella virus, the orthomyxoviruses, including Type A, B, and C influenza viruses, the bunyaviruses, including the Rift Valley fever virus and the hantavirus, the filoviruses such as Ebola virus and Marburg virus, and the paramyxoviruses, including mumps virus and measles virus.

11. Effective Dose

Toxicity and therapeutic efficacy of such compounds can be determined by standard pharmaceutical procedures in cell cultures or experimental animals, e.g., for determining The Ld₅₀ (The Dose Lethal To 50% Of The Population) And The Ed₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

12. Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ation oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated, in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the oligomers of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included.

Systemic administration can also be by transmucosal or transdermal means, or the compounds can be administered orally. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as generally known in the art.

### EXAMPLES

1. Role of PRT3 in Virus-Like Particle (VLP) Budding

1. Objective:

Use RNAi to inhibit PRT3 gene expression and compare the efficiency of viral budding and Gag expression and processing in treated and untreated cells.

2. Study Plan:

HeLa SS-6 cells are transfected with mRNA-specific RNAi in order to knockdown the target proteins. Since maximal reduction of target protein by RNAi is achieved after 48 hours, cells are transfected twice—first to reduce target mRNAs, and subsequently to express the viral Gag protein. The second transfection is performed with pNLenv (plasmid that encodes HIV) and with low amounts of RNAi to maintain the knockdown of target protein during the time of gag expression and budding of VLPs. Reduction in mRNA levels due to RNAi effect is verified by RT-PCR amplification of target mRNA.

3. Methods, Materials, Solutions

a. Methods
i. Transfections according to manufacturer's protocol and as described in procedure.
ii. Protein determined by Bradford assay.
iii. SDS-PAGE in Hoeffer miniVE electrophoresis system. Transfer in Bio-Rad mini-protean II wet transfer system. Blots visualized using Typhoon system, and ImageQuant software (ABbiotech)

b. Materials

| Material | Manufacturer | Catalog # | Batch # |
|---|---|---|---|
| Lipofectamine 2000 | Life Technologies | 11668-019 | 1112496 |
| (LF2000) | | | |
| OptiMEM | Life Technologies | 31985-047 | 3063119 |
| RNAi Lamin A/C | Self | 13 | |
| RNAi TSG101 688 | Self | 65 | |
| RNAi PRT3 524 | Self | 81 | |
| plenvl1 PTAP | Self | 148 | |
| plenvl1 ATAP | Self | 149 | |
| Anti-p24 polyclonal | Seramun | | A-0236/5- |
| antibody | | | 10-01 |
| Anti-Rabbit Cy5 | Jackson | 144-175-115 | 48715 |
| conjugated antibody | | | |
| 10% acrylamide Tris- | Life Technologies | NP0321 | 1081371 |
| Glycine SDS-PAGE gel | | | |
| Nitrocellulose | Schleicher & | 401353 | BA-83 |
| membrane | Schuell | | |
| NuPAGE 20× transfer | Life Technologies | NP0006-1 | 224365 |
| buffer | | | |
| 0.45 μm filter | Schleicher & | 10462100 | CS1018-1 |
| | Schuell | | |

c. Solutions

| | Compound | Concentration |
|---|---|---|
| Lysis Buffer | Tris-HCl pH 7.6 | 50 mM |
| | MgCl₂ | 15 mM |
| | NaCl | 150 mM |
| | Glycerol | 10% |
| | EDTA | 1 mM |
| | EGTA | 1 mM |
| | ASB-14 (add immediately | 1% |
| | before use) | |
| 6 × Sample | Tris-HCl, pH = 6.8 | 1M |
| Buffer | Glycerol | 30% |
| | SDS | 10% |
| | DTT | 9.3% |
| | Bromophenol Blue | 0.012% |
| TBS-T | Tris pH = 7.6 | 20 mM |
| | NaCl | 137 mM |
| | Tween-20 | 0.1% |

4. Procedure

a. Schedule

Day

| 1 | 2 | 3 | 4 | 5 |
|---|---|---|---|---|
| Plate | Transfection I | Passage | Transfection II | Extract RNA |
| cells | (RNAi only) | cells | (RNAi and pNlenv) | for RT-PCR |
| | | (1:3) | (12:00, PM) | (post |
| | | | | transfection) |
| | | | Extract RNA for | Harvest VLPs |
| | | | RT-PCR | and cells |
| | | | (pre-transfection) | |

b. Day 1

Plate HeLa SS-6 cells in 6-well plates (35 mm wells) at concentration of 5×10⁵ cells/well.

c. Day 2

2 hours before transfection replace growth medium with 2 ml growth medium without antibiotics.

Transfection I:

| | | | | | RNAi | A | B |
|---|---|---|---|---|---|---|---|
| | | | | | [20 μM] | OPtiMEM | LF2000 mix |
| Reaction | RNAi name | TAGDA# | Reactions | RNAi [nM] | μl | (μl) | (μl) |
| 1 | Lamin A/C | 13 | 2 | 50 | 12.5 | 500 | 500 |
| 2 | Lamin A/C | 13 | 1 | 50 | 6.25 | 250 | 250 |
| 3 | TSG101 688 | 65 | 2 | 20 | 5 | 500 | 500 |
| 5 | PRT3 524 | 81 | 2 | 50 | 12.5 | 500 | 500 |

Transfections:

Prepare LF2000 mix: 250 μl OptiMEM+5 μl LF2000 for each reaction. Mix by inversion, 5 times. Incubate 5 minutes at room temperature.

Prepare RNA dilution in OptiMEM (Table 1, column A). Add LF2000 mix dropwise to diluted RNA (Table 1, column B). Mix by gentle vortex. Incubate at room temperature 25 minutes, covered with aluminum foil.

Add 500 μl transfection mixture to cells dropwise and mix by rocking side to side. Incubate overnight.

d. Day 3

Split 1:3 after 24 hours. (Plate 4 wells for each reaction, except reaction 2 which is plated into 3 wells.)

e. Day 4

2 hours pre-transfection replace medium with DMEM growth medium without antibiotics.

Transfection II

| | | | | | | A | B | | |
|---|---|---|---|---|---|---|---|---|---|
| | | | | | | Plasmid | RNAi [20 μM] | C | D |
| | | | | | Plasmid | for 2.4 μg | for 10 nM | OPtiMEM | LF2000 mix |
| Reaction | RNAi name | TAGDA# | Plasmid | Reactions | (μg/μl) | (μl) | (μl) | (μl) | (μl) |
| 1 | Lamin A/C | 13 | PTAP | 3 | | 3.4 | 3.75 | 750 | 750 |
| 2 | Lamin A/C | 13 | ATAP | 3 | | 2.5 | 3.75 | 750 | 750 |
| 3 | TSG101 688 | 65 | PTAP | 3 | | 3.4 | 3.75 | 750 | 750 |
| 5 | PRT3 524 | 81 | PTAP | 3 | | 3.4 | 3.75 | 750 | 750 |

Prepare LF2000 mix: 250 μl OptiMEM+5 μl LF2000 for each reaction. Mix by inversion, 5 times. Incubate 5 minutes at room temperature.

Prepare RNA+DNA diluted in OptiMEM (Transfection II, A+B+C)

Add LF2000 mix (Transfection II, D) to diluted RNA+DNA dropwise, mix by gentle vortex, and incubate 1 h while protected from light with aluminum foil.

Add LF2000 and DNA+RNA to cells, 500 μl/well, mix by gentle rocking and incubate overnight.

f. Day 5

Collect samples for VLP assay (approximately 24 hours post-transfection) by the following procedure (cells from one well from each sample is taken for RNA assay, by RT-PCR).

g. Cell Extracts
i. Pellet floating cells by centrifugation (5 min, 3000 rpm at 40° C.), save supernatant (continue with supernatant immediately to step h), scrape remaining cells in the medium which remains in the well, add to the corresponding floating cell pellet and centrifuge for 5 minutes, 1800 rpm at 40° C.
ii. Wash cell pellet twice with ice-cold PBS.
iii. Resuspend cell pellet in 100 μl lysis buffer and incubate 20 minutes on ice.
iv. Centrifuge at 14,000 rpm for 15 min. Transfer supernatant to a clean tube. This is the cell extract.
v. Prepare 10 μl of cell extract samples for SDS-PAGE by adding SDS-PAGE sample buffer to 1×, and boiling for 10 minutes. Remove an aliquot of the remaining sample for protein determination to verify total initial starting material. Save remaining cell extract at −80° C.
h. Purification of VLPs from Cell Media
i. Filter the supernatant from step g through a 0.45 m filter.
ii. Centrifuge supernatant at 14,000 rpm at 40 C for at least 2h.
iii. Aspirate supernatant carefully.
iv. Re-suspend VLP pellet in hot (100° C. warmed for 10 min at least) 1× sample buffer.
v. Boil samples for 10 minutes, 1001C.

i. Western Blot analysis
i. Run all samples from stages A and B on Tris-Glycine SDS-PAGE 10% (120V for 1.5 h.).
ii. Transfer samples to nitrocellulose membrane (65V for 1.5h.).
iii. Stain membrane with ponceau S solution.
iv. Block with 10% low fat milk in TBS-T for 1 h.
v. Incubate with anti p24 rabbit 1:500 in TBS-T o/n.
vi. Wash 3 times with TBS-T for 7 min each wash.
vii. Incubate with secondary antibody anti rabbit cy5 1:500 for 30 min.
viii. Wash five times for 10 min in TBS-T
ix. View in Typhoon gel imaging system (Molecular Dynamics/APBiotech) for fluorescence signal.

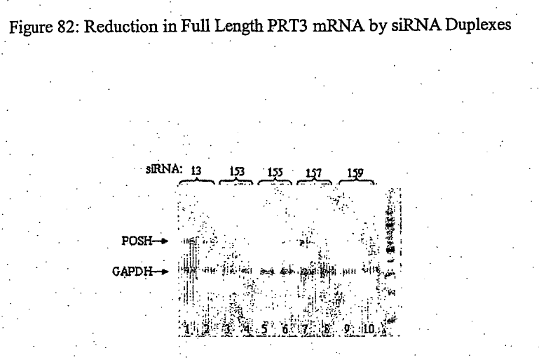

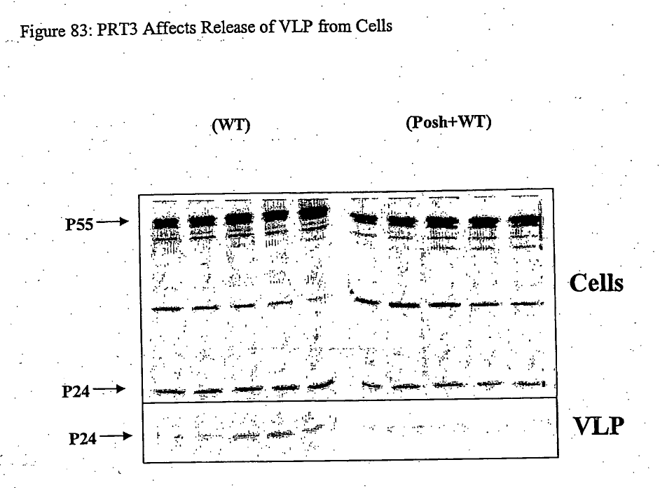

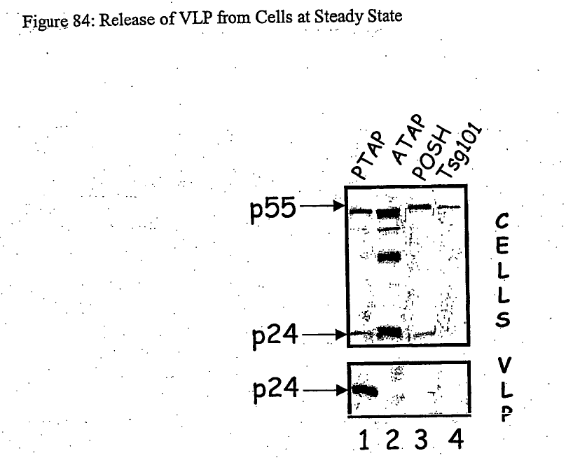

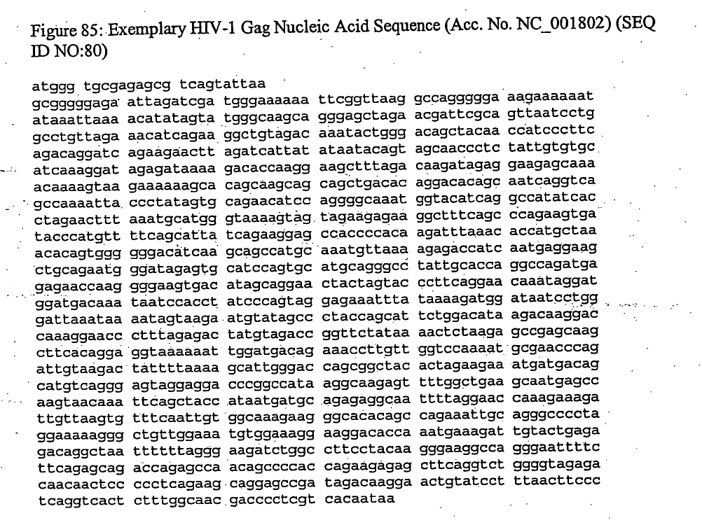

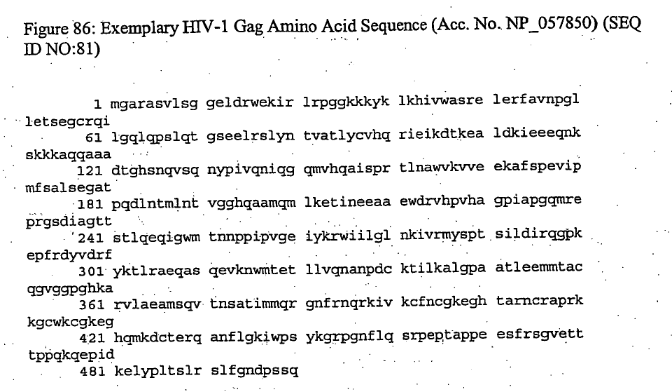

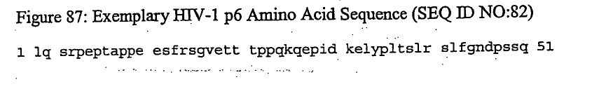

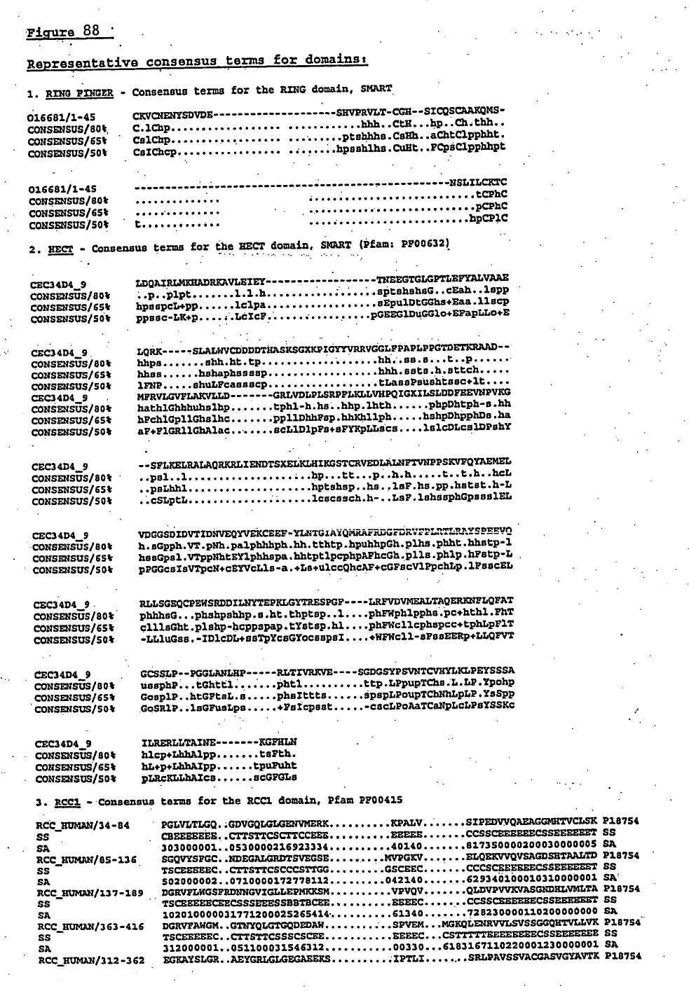

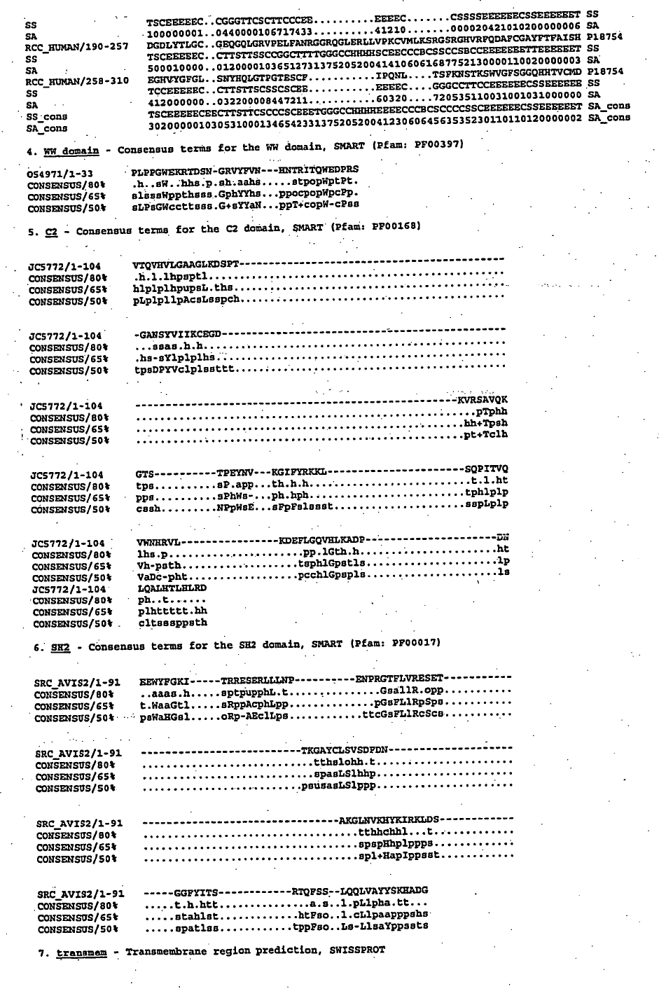

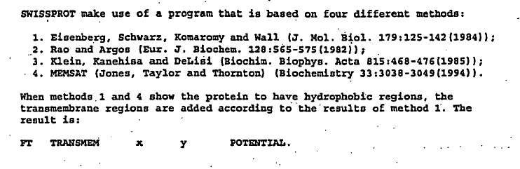

Results are shown in FIGS. 82-84.

2. Exemplary PRT3 RT-PCR primers and siRNA duplexes

RT-PCR Primers

| | | |
|---|---|---|
| Name | Position | Sequence |

| | | | |
|---|---|---|---|
| Sense primer | PRT3═271 | 271 | 5′ CTTGCCTTGCCAGCATAC 3′ (SEQ ID NO:12) |
| Anti-sense | PRT3═926c | 926C | 5′ CTGCCAGCATTCCTTCAG 3′ (SEQ ID NO:13) |
| primer | | | |

siRNA Duplexes:

| | | |
|---|---|---|
| siRNA No: | 153 | |
| siRNA Name: | PRT3-230 | |
| Position in mRNA | 426-446 | |
| Target sequence: | 5′ AACAGAGGCCTTGGAAACCTG 3′ | SEQ ID NO: 14 |
| siRNA sense strand: | 5′ dTdTCAGAGGCCUUGGAAACCUG 3′ | SEQ ID NO: 15 |
| siRNA anti-sense strand: | 5′dTdTCAGGUUUCCAAGGCCUCUG 3′ | SEQ ID NO: 16 |
| siRNA No: | 155 | |
| siRNA Name: | PRT3-442 | |
| Position in mRNA | 638-658 | |
| Target sequence: | 5′ AAAGAGCCTGGAGACCTTAAA 3′ | SEQ ID NO: 17 |
| siRNA sense strand: | 5′ ddTdTAGAGCCUGGAGACCUUAAA 3′ | SEQ ID NO: 18 |
| siRNA anti-sense strand: | 5′ ddTdTUUUAAGGUCUCCAGGCUGU 3′ | SEQ ID NO: 19 |
| siRNA No: | 157 | |
| siRNA Name: | PRT3-U111 | |
| Position in mRNA | 2973-2993 | |
| Target sequence: | 5′ AAGGATTGGTATGTGACTCTG 3′ | SEQ ID NO: 20 |
| siRNA sense strand: | 5′ dTdTGGAUUGGUAUGUGACUCUG 3′ | SEQ ID NO: 21 |
| siRNA anti-sense strand: | 5′ dTdTCAGAGUCACAUACCAAUCC 3′ | SEQ ID NO: 22 |
| siRNA No: | 159 | |
| siRNA Name: | PRT3-U410 | |
| Position in mRNA | 3272-3292 | |
| Target sequence: | 5′ AAGCTGGATTATCTCCTGTTG 3′ | SEQ ID NO: 23 |
| siRNA sense strand: | 5′ ddTdTGCUGGAUUAUCUCCUGUUG 3′ | SEQ ID NO: 24 |
| siRNA anti-sense strand: | 5′ ddTdTCAACAGGAGAUAAUCCAGC 3′ | SEQ ID NO: 25 |

3. Effects of PRT3 RNAi on HIV Release: Kinetics

A1. Transfections
1. One day before transfection plate cells at a concentration of 5×10⁶ cell/well in 15 cm plates.
2. Two hours before transfection, replace cell media to 20 ml complete DMEM without antibiotics.
3. DNA dilution: for each transfection dilute 62.511 RNAi in 2.5 ml OptiMEM according to the table below. RNAi stock is 201M (recommended concentration: 50 nM, dilution in total medium amount 1:400).
4. LF 2000 dilution: for each transfection dilute 50 μl LF 2000 reagent in 2.5 ml OptiMEM.
5. Incubate diluted RNAi and LF 2000 for 5 minutes at RT.
6. Mix the diluted RNAi with diluted LF2000 and incubated for 20-25 minutes at RT.
7. Add the mixture to the cells (drop wise) and incubate for 24 hours at 37° C. in CO₂ incubator.
8. One day after RNAi transfection split cells (in complete MEM medium to 2 15 cm plate and 1 well in a 6 wells plate)
9. One day after cell split perform HIV transfection.
10. 6 hours after HIV transfection replace medium to complete MEM medium.
RT-PCR for PRT3 may be performed to assess the degree of knockdown.

A2. Total RNA Purification.
1. One day after transfection, wash cells twice with sterile PBS.

2. Scrape cells in 2.3 ml/200 μl (for 15 cm plate/1 well of a 6 wells plate) Tri reagent (with sterile scrapers) and freeze in −70° C.

| | Chase time | | |
|---|---|---|---|
| Treatment | (hours) | Fraction | Labeling |
| Control = WT | 1 | Cells | A1 |
| | | VLP | A1 V |
| | 2 | Cells | A2 |
| | | VLP | A2 V |
| | 3 | Cells | A3 |
| | | VLP | A3 V |
| | 4 | Cells | A4 |
| | | VLP | A4 V |
| | 5 | Cells | A5 |
| | | VLP | A5 V |
| PRT3 + WT | 1 | Cells | B1 |
| | | VLP | B1 V |
| | 2 | Cells | B2 |
| | | VLP | B2 V |
| | 3 | Cells | B3 |
| | | VLP | B3 V |
| | 4 | Cells | B4 |
| | | VLP | B4 V |
| | 5 | Cells | B5 |
| | | VLP | B5 V |

B. Labeling
1. Take out starvation medium, thaw and place at 37° C.
2. Scrape cells in growth medium and transfer gently into 15 ml conical tube.
3. Centrifuge to pellet cells at 1800 rpm for 5 minutes at room temperature.
4. Aspirate supernatant and let tube stand for 10 sec. Remove the rest of the supernatant with a 200%1 pipetman.
5. Gently add 10 ml warm starvation medium and resuspend carefully with a 10 ml pipette, up and down, just turning may not resolve the cell pellet).
6. Transfer cells to 10 cm tube and place in the incubator for 60 minutes. Set an Eppendorf thermo mixer to 37° C.
7. Centrifuge to pellet cells at 1800 rpm for 5 minutes at room temperature.
8. Aspirate supernatant and let tube stand for 10 sec. Remove the rest of the supernatant with a 200 μl pipetman.
9. Cut a 2001 μl tip from the end and resuspend cells (˜1.5 10⁷ cells in 150 μl RPIM without Met, but try not to go over 250 μl if you have more cells) gently in 1501 μl starvation medium. Transfer cells to an Eppendorf tube and place in the thermo mixer. Wait 10 sec and transfer the rest of the cells from the 10 ml tube to the Eppendorf tube, if necessary add another 50 μl to splash the rest of the cells out (all specimens should have the same volume of labeling reaction).
10. Pulse: Add 50 μl of ³⁵S-methionine (specific activity 14.2 μCi/μl), tightly cup tubes and place in thermo mixer. Set the mixing speed to the lowest possible (700 rpm) and incubate for 25 minutes.
11. Stop the pulse by adding 1 ml ice-cold chase/stop medium. Shake tube very gently three times and pellet cells at 600 rpm for 6 sec.
12. Remove supernatant with a 1 ml tip. Add gently 1 ml ice-cold chase/stop medium to the pelleted cells and invert gently to resuspend.
13. Chase: Transfer all tubes to the thermo mixer and incubate for the required chase time (830: 1, 2, 3, 4 and 5 hours; 828: 3 hours only). At the end of total chase time, place tubes on ice, add 1 ml ice-cold chase/stop and pellet cells for 1 minute at 14,000 rpm. Remove supernatant and transfer supernatant to a second eppendorf tube. The cell pellet freeze at −80° C., until all tubes are ready.
14. Centrifuge supernatants for 2 hours at 14,000 rpm, 4° C. Remove the supernatant very gently, leave 20 μl in the tube (labeled as V) and freeze at −80° C. until the end of the time course.
*** All steps are done on ice with ice-cold buffers
15. When the time course is over, remove all tubes form −80° C. Lyse VLP pellet (from step 14) and cell pellet (step 13) by adding 500 μl of lysis buffer (see solutions), resuspend well by pipeting up and down three times. Incubate on ice for 15 minutes, and spin in an eppendorf centrifuge for 15 minutes at 4° C., 14,000 rpm. Remove supernatant to a fresh tube, discard pellet.

16. Perform IP with anti-p24 sheep for all samples.

C. Immunoprecipitation
1. Preclearing: add to all samples 15 μl ImmunoPure PlusG (Pierce). Rotate for 1 hour at 4° C. in a cycler, spin 5 min at 4° C., and transfer to a new tube for IP.
2. Add to all samples 20 μl of p24-protein G conjugated beads and incubate 4 hours in a cycler at 4° C.
3. Post immunoprecipitations, transfer all immunoprecipitations to a fresh tube.
4. Wash beads once with high salt buffer, once with medium salt buffer and once with low salt buffer. After each spin don't remove all solution, but leave 50 μl solution on the beads. After the last spin remove supernatant carefully with a loading tip and leave ˜10 μl solution.
5. Add to each tube 20 μl 2×SDS sample buffer. Heat to 70° C. for 10 minutes.
6. Samples were separated on 10% SDS-PAGE.
7. Fix gel in 25% ethanol and 10% acetic acid for 15 minutes.
8. Pour off the fixation solution and soak gels in Amplify solution (NAMP 100 Amersham) for 15 minutes.
9. Dry gels on warm plate (60-80° C.) under vacuum.
10. Expose gels to screen for 2 hours and scan.
   4. Prophetic Characterization of Viral Maturation Polypeptides and Systems

The methods disclosed herein are useful for, among other things, identifying host proteins involved in virus release. To this end we compare membrane protein profiles of cells infected with viruses expressing wild type (wt) p6Gag (p6) with the parallel profiles from cells infected with viruses harboring a mutant form of p6. Wt p6 is expected to attract the release machinery and it is expected that the mutant virus fails to do so. Furthermore, we also investigate the mechanism of the exceedingly high efficiency release mechanism of the Ebola virus by comparing the protein profiles of membranes from cells infected with mutant HIV-1 expressing the Ebola release determinant with membranes of cells infected with wt virus.

Host Protein Profiling

Membranes from uninfected, wt virus and mutant virus-infected cells are prepared according to protocols modified to enable virus inactivation prior to sample handling and separation. The membranes samples are separated by 2D gel electrophoresis (2DGE). The 2D maps are analyzed and proteins specific to wt virus are subjected to mass spectrometry analysis.

Virion Protein Profiling

Host proteins involved in virus release are expected to be trapped in virions after their release. This expectation is based, in part, on two observations. The first is the presence of ubiquitin in virions at concentrations higher than the ubiquitin cellular concentration. The second is the finding that EIAV gag protein associates with AP50 and that AP-2 is also found in EIAV particles. We have found mono-ubiquitinated p9 of EIAV in the virions.

It is therefore possible that additional host proteins are included in virions. Analysis of host proteins included in virus particles facilitates identification of host proteins involved in virus budding and release.

To identify host proteins included in virus particles, we harvest the virions from the supernatant of virus-infected cells. The virions are then by lysed and the proteins are subsequently analyzed by 2DGE. Specific antibodies are used to identify the viral proteins. The unidentified host proteins are subjected to MS analysis. Antibodies to the host proteins present in the virus particles are used to detect them in membranes of virus infected cells. It is believed that host proteins present in virions and localization in sites of virus budding will be involved in virus maturation.

Identification of Ubiquitinated Proteins Associated with Virus Release

Cells transfected with hemaglutinin (HA)-tagged ubiquitin are infected with the relevant virus. To isolate the ubiquitinated proteins, detergent lysates are prepared and the detergent extract is subjected to immunoprecipitation with anti-HA antibody. The immunoprecipitates are subjected to separation by either SDS-PAGE or 2DGE (depending on the complexity of the proteome). Using this approach we compare ubiquitinated proteins from wt and mutant virus-infected cells. Those proteins that are ubiquitinated in wt but not in mutant-infected cells are identified and characterized by mass spectrometry analysis.

Identification of Ubiquitin-Protein Ligases

The rate-limiting component for virus release is expected to be a ubiquitin-protein ligase. The Ebola recruits the ligase to the sites of budding with exceedingly higher efficiency than any other retrovirus.
1. We generate a recombinant HIV1-p6 with the Ebola tandem L motif. To confirm the Ebola potency we compare virus-like particle release into the medium of cells expressing the two p6 forms. Virus-like particles are harvested from the medium of p6-expressing cells. We quantitatively detect Gag with specific antibodies by immunoblot analysis. It is expected that Gag signal will be much higher in the supernatant of p6 Ebola expressing cells.
2. We prepare membraned protein-enriched fraction. To this end we prepare protein fractions from unwashed or mildly washed membrane so as to minimally disturb possible ligase-protein interactions. Protein profiles of plasma membrane proteins from un-infected, p6 _{HIV-1} and P6 _{Ebola}-transfected cells are compared. Proteins that are expressed at higher levels in the P6 Ebola membranes are analyzed and identified by mass spectrometry. We specifically search for proteins with the characteristics of an AVMSP.
3. Alternatively, we utilize anti-p6 antibodies to precipitate proteins that are associated with p6. A 2D profile of p6-associated proteinsis performed and results are analyzed via a similar rational as described in part 2 above.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. An isolated protein complex comprising a RING-SH₃ polypeptide in combination with at least one polypeptide selected from the group consisting of: a Gag protein, a Gag late domain, PI3K, actin, myosin, Hsp60, Hsp70, Hsp90, STAM1, STAM2A, STAM2B, VHS-UIM, a GTPase, an E2 enzyme, tsg101, a cullin, RING-SH₃, and a clathrin.

2. The isolated protein complex of claim 1, wherein said Gag protein is an HIV gag protein.

3. The isolated protein complex of claim 1, wherein said Gag protein comprises the Gag late domain.

4. The isolated protein complex of claim 3, wherein said Gag late domain is PTAP.

5. The isolated protein complex of claim 3, wherein said Gag late domain is PxxY.

6. The isolated protein complex of claim 3, wherein said Gag late domain is PxxL.

7. The isolated protein complex of claim 3, wherein said Gag late domain is PPxY.

8. The isolated protein complex of claim 3, wherein said Gag late domain is YxxL.

9. The isolated protein complex of claim 3, wherein said Gag late domain is PxxP.

10. A host cell comprising a first nucleic acid and a second nucleic acid, wherein the first nucleic acid comprises a recombinant RING-SH₃ nucleic acid, and wherein the second nucleic acid comprises a recombinant nucleic acid encoding a Gag protein.

11. The host cell of claim 10, wherein said Gag protein is an HIV gag protein.

12. The host cell of claim 11, wherein said Gag protein comprises the Gag late domain.

13. The host cell of claim 12, wherein said Gag late domain is PTAP.

14. The host cell of claim 12, wherein said Gag late domain is PxxY.

15. The host cell of claim 12, wherein said Gag late domain is PxxL.

16. The host cell of claim 12, wherein said Gag late domain is PPxY.

17. The host cell of claim 12, wherein said Gag late domain is YxxL.

18. The host cell of claim 12, wherein said Gag late domain is PxxP.

19. A method for identifying modulators of protein complexes, comprising:
(i) forming a reaction mixture comprising
(a) a RING-SH₃; and
(b) a second polypeptide selected from the group consisting of: RING-SH₃, a gag protein, a Gag late domain, PI3K, actin, myosin, Hsp60, Hsp70, Hsp90, STAM1, STAM2A, STAM2B, VHS-UIM, a GTPase, an E2 enzyme, tsg101, a cullin, and a clathrin;
(ii) contacting said reaction mixture with a test agent, and
(iii) determining the effect of said test agent for one or more activities selected from the group comprising (a) a change in the level of the protein complex, (b) a change in the enzymatic activity of the complex, or (c) where the reaction mixture is a whole cell, a change in the plasma membrane localization of the complex or a component thereof.

20. A method for identifying a test compound which inhibits or potentiates complex formation, comprising:
(i) forming a reaction mixture comprising
(a) a RING-SH₃; and
(b) a second polypeptide selected from the group consisting of: RING-SH₃, a gag protein, a Gag late domain, P13K, actin, myosin, Hsp60, Hsp70, Hsp90, STAM1, STAM2A, STAM2B, VHS-UIM, a GTPase, an E2 enzyme, tsg101, a cullin, and a clathrin;
(ii) contacting said reaction mixture with a test agent, and
(iii) detecting binding of said RING-SH₃ to said second polypeptide;
wherein a change in the binding of said RING-SH₃ to said second polypeptide in the presence of the test compound, relative to binding in the absence of the test compound, indicates that said test compound potentiates or inhibits complex formation between said RING-SH₃ and said second polypeptide.

21. A method for inhibiting infection in a subject in need thereof, comprising administering an effective amount of an agent that inhibits the binding of a RING-SH₃ polypeptide to an gag protein.

22. The method of claim 21, wherein said agent is selected from the group comprising a small molecule, a antibody, and a peptide.

23. The method of claim 22, wherein the Gag protein is an HIV Gag protein.

24. The method of claim 22, wherein the Gag polypeptide is HIV p24.

25. An isolated antibody, or fragment thereof, specifically immunoreactive with an epitope of a RING-SH₃ polypeptide, which disrupts the interaction between said RING-SH₃ and a RING-SH₃-associating polypeptide (RING-SH₃-AP).

26. The antibody of claim 25, wherein said antibody is a monoclonal antibody.

27. The antibody of claim 25, wherein said antibody is a Fab fragment.

28. The antibody of claim 25, wherein said antibody is labeled with a detectable label.

29. The antibody of claim 25, wherein said RING-SH₃-AP is a gag polypeptide.

30. The antibody of claim 25, wherein said RING-SH₃-AP is an HIV gag polypeptide.

31. A kit for detecting a RING-SH₃ polypeptide protein comprising (i) isolated anti-RING-SH₃ antibodies, or fragment thereof, specifically immunoreactive with an epitope of an RING-SH₃, which epitope interacts with an RING-SH₃-AP, and (ii) a detectable label for detecting said anti-RING-SH₃ antibody in immunoclomplexes with said RING-SH₃ polypeptide.

32. A host cell comprising a first nucleic acid and a second nucleic acid, wherein the first nucleic acid comprises a recombinant RING-SH₃ nucleic acid, and wherein the second nucleic acid comprises a recombinant nucleic acid encoding a HIV Gag protein.

33. A method for inhibiting infection in a subject in need thereof, comprising administering an effective amount of an agent that inhibits the binding of a RING-SH₃ polypeptide to an HIV gag protein.

34. An anti-viral therapeutic composition comprising a double stranded oligoribonucleotide molecule that inhibits expression of a nucleic acid molecule encoding a RING-SH₃ polypeptide
